(19) United States
(12) Patent Application Publication
(10) Pub. No.: US 2010/0077500 A1
(43) Pub. Date: Mar. 25, 2010

(54) SCREENING FOR VASE-LIFE AND FOR RESISTANCE TO XANTHOMONAS IN ANTHURIUM

(75) Inventors: Pathmanathan Umaharan, Maracas (LK); Winston Elibox, Castries (LC)

Correspondence Address: WILMERHALE/BOSTON, 60 STATE STREET, BOSTON, MA, 02109, US

(73) Assignee: University of the West Indies, a Regional Instutio established by Royal Charter, Kingston (JM)

(21) Appl. No.: 12/445,045

(22) PCT Filed: Oct. 10, 2007
(86) PCT No.: PCT/IB07/04677

§ 371 Date: Apr. 9, 2009

Related U.S. Application Data
(60) Provisional application No. 60/850,955, filed on Oct. 10, 2006.

(51) Int. Cl. A01H 1/02; C12Q 1/02; C12N 1/20; A01H 5/00

(52) U.S. Cl. 800/260; 435/29; 435/252.1; 800/298

(57) ABSTRACT

Anthurium species are important ornamental crops that are susceptible to infection by Xanthomonas campestris pv. Dieffenbachiae. Methods of identifying target plants that are resistant to or tolerant of Xanthomonas infection are described. Methods of predicting vase-life performance are also disclosed.

### CROSS-REFERENCE TO RELATED APPLICATIONS

This application claims priority to provisional U.S. Application Ser. No. 60/850,955, filed on Oct. 10, 2006, which is herein incorporated by reference in its entirety.

### TECHNICAL FIELD

This application relates to the field of plant disease, and more particularly to disease resistance and vase-life in Anthurium.

### BACKGROUND

Anthurium andraeanum (Hort.) is an important export ornamental crop in the Caribbean. The anthurium industry, during the past decade, has been debilitated by the bacterial blight disease caused by Xanthomonas campestris pv. Dieffenbachiae (X. axonopodis pv. Dieffenbachia). Cultivated anthurium belongs to two sections viz. Calomystrium and Porphyrochitonium, of the 18 sections. The section, Calomystrium includes the popular Anthurium andraeanum (Linden) ex André, the best known among the cultivated anthurium species.

The anthurium cut-flower comprises an inflorescence (spadix) subtended by a modified leaf (spathe) borne on a long stalk (peduncle).

Progress in breeding for resistance to the bacterial blight disease has been hampered globally, due to lack of suitable screening methods and a poor understanding of the genetics of resistance.

Currently, the anthurium industry in the Caribbean region is a risky and costly proposition. High costs are incurred in disease control, obtaining planting material and establishing screen-houses. Furthermore, many Dutch cultivars grown in the region are highly susceptible to two bacterial diseases viz. bacterial blight disease caused by Xanthomonas campestris pv. dieffenbachiae (Bradbury, 1986, Guide to Plant Pathogenic Bacteria, pp. 198-260) and bacterial leaf spot disease caused by Acidovorax anthurii.

Cultivar susceptibility to bacterial blight disease is determined by at least two phase-differential mechanisms (Fukui et al, 1998, Plant Dis., 82(7):800-806). Management of bacterial blight disease is costly and involves fumigation of beds, prophylactic spraying of chemicals, and physical sanitation. Despite these measures, epidemics have destroyed many farms in the Caribbean, especially in Jamaica and Trinidad where several farms have closed operations during the past decade. Systemic infection can occur independently of foliar infections (Prior et al, 1985, Agronomie Tropicale 42:61-68; Bureau IMAC Bleiswijk, 1998, 2665 KV Bleiswijk, the Neterlands: Bureau IMAC; Fukui et al, 1998, Plant Disease, 82(7):800-806). In such cases, the pathogen enters the plant via wounds (Nishijimi, 1988, Proc. First Anthurium Blight Conf., 02:08:88; Fernandez et al, 1989, Proc. Second Anthurium Blight Conf., 03:10:89; Fukui et al, 1998, Plant Dis., 82(7):800-806) or through the root system (Prior et al, 1985, Agronomie Tropicale 42:61-68). The disease advances without apparent symptoms (Tanabe et al, 1994, Proc. Fifth Hawaii Anthurium Industry Conf. 02:02:94) until the infected plant wilts and collapses (Fernandez et al, 1991, Proc. Fourth Hawaii Anthurium Indus. Conf., pp. 10-11; Fukui et al, 1998, Plant Dis., 82(7):800-806).

A systematic anthurium breeding effort aimed at exploiting the genetic potential within the local and imported germplasm of anthurium in the Caribbean requires the collection and characterization of existing germplasm. No studies have evaluated the existing cultivars in the Caribbean for their adaptability and productivity. Furthermore, there have been only limited genetic studies outlining the genetic basis of some of the important characteristics sought in breeding such as resistance to bacterial diseases, vase-life, productivity and cut-flower characteristics. Moreover, practical, efficient and reliable screening methodologies are required for these characteristics. Such information is necessary to develop an effective and efficient breeding program to develop elite, tropically adapted cultivars that can rescue the anthurium industry in the Caribbean and other regions with an anthurium industry.

### SUMMARY

The invention relates to screening methods to assess foliar and systemic resistance to bacterial blight disease, for example, in Anthurium. The invention also relates to methods of screening for vase-life and other horticultural characteristics. Such methods can be used, for example, to identify elite parental cultivars from a germplasm collection.

Accordingly, the invention includes a method of identifying resistance or tolerance of a plant to Xanthomonas infection, e.g., systemic or foliar resistance or tolerance. In some aspects, the method detects systemic resistance or tolerance includes providing a target plant, e.g., an anthurium; infecting the target plant with a fluorescent Xanthomonas expressing a heterologous fluorescent protein; and detecting the level of fluorescent protein in the target plant, such that a level of fluorescent protein in the target plant that is less than the level in a susceptible target plant indicates that the target plant is resistant to or tolerant of infection by the Xanthomonas. In some cases, the Xanthomonas is Xanthomonas campestris pv. dieffenbachiae. The fluorescent protein can be, for example, Green Fluorescent Protein. In certain embodiments of the method, the Xanthomonas comprises p519 ngfp. In some cases, the method detects systemic resistance and foliar resistance, e.g., such as using a leaf-disc vacuum-infiltration method to determine foliar infection.

The invention also relates to a method of identifying resistance or tolerance of a plant to Xanthomonas infection in which foliar resistance is determined. The method includes; providing a leaf sample from a target plant, infecting the leaf sample with a Xanthomonas, detecting Xanthomonas infection in the infected leaf sample, and comparing the rate of infection to a reference to determine foliar resistance of the target plant.

In another aspect, the invention relates to a recombinant X. campestris comprising a fluorescent protein, e.g., a Green Fluorescent Protein. In some embodiments, the recombinant X. campestris comprises p519 ngfp.

In some aspects, the invention relates to a method of predicting vase-life performance of an anthurium. The method includes; providing a sample from an anthurium; determining abaxial stomata number and spathe color in the anthurium sample; and comparing abaxial stomata number and spathe color to a control or reference set, thereby predicting vase-life performance of the anthurium.

In yet another aspect, the invention relates to a method of predicting vase-life performance of an anthurium that includes; providing an anthurium; determining the average time for spadix necrosis of the anthurium; and comparing the average time for spadix necrosis of the anthurium to a reference, thereby predicting the vase-life performance of the anthurium.

The invention disclosed herein also relates to a plant breeding program that includes at least one of; assaying cultivars for resistance to Xanthomonas infection (e.g., resistance to systemic infection, resistance to foliar infection, or resistance to both systemic and foliar infection); and selecting resistant cultivars as a parental cultivar in the breeding program; assaying cultivars for vase-life using a method described herein and selecting a cultivar having a vase-life of at least three weeks as a parental cultivar in the breading program; or assaying cultivars for at least one type of resistance to Xanthomonas and assaying the cultivars for vase-life.

In another aspect, the invention includes a method of selecting an anthurium cultivar for propagation or a breeding program by assaying at least one cultivar for foliar and systemic resistance to Xanthomonas and selecting at least one cultivar for propagation or a breeding program that has displays both systemic and foliar resistance to Xanthomonas.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description and from the claims.

### BRIEF DESCRIPTION OF THE DRAWINGS

FIG. 1 is a graph depicting a sigmoid growth curve of Xanthomonas campestris pv. dieffenbachiae in Trinidad. Differences between the three replicates were not significant at P≦0.05.

FIG. 2 is a graph depicting the relationship between field resistance rankings for 22 anthurium cultivars and systemic resistance to bacterial blight disease based on the time taken to death obtained from a screening experiment in the dry season. Cultivars that did not die at all were given a time of 40 weeks to death to separate them clearly from the others.

FIG. 3 is a graph depicting the relationship between field resistance rankings for 22 cultivars and systemic resistance to bacterial blight disease based on the proportion of dead plants from a screening experiment in the dry season. 1=‘Acropolis,’ 2=‘Ibara,’ 3=‘Local Orange,’ 4=‘Mirjam,’ 5=‘Tropical,’ 6=‘Senator,’ 7=‘Venus,’ 8=‘Walapana,’ 9=‘Spirit,’ 10=‘Honduras,’ 11=‘Sonate,’ 12=‘Success,’ 13=‘Rosa,’ 14=‘Lydia,’ 15=‘Champagne,’ 16=‘Pierrot,’ 17=‘Gloria,’ 18=‘Cheers,’ 19=‘Midori,’ 20=‘Cuba,’ 21=‘Hawaii,’ and 22=‘Fantasia’.

FIG. 4 is a graph depicting the frequency distribution for time to death of 56 anthurium cultivars subjected to artificial screening against Xanthomonas campestris pv. dieffenbachiae. Cultivars with no plants that died were given a dummy value of 30 to distinguish them from the other categories.

FIG. 5 is a bar graph depicting the frequency distribution for proportion of dead plants of 56 anthurium cultivars subjected to artificial screening against Xanthomonas campestris pv. dieffenbachiae.

FIG. 6 is a graph depicting the relationship between TLS (days) in the early vs. late wet season for 14 Anthurium andraeanum (Hort.) cultivars evaluated for foliar resistance to bacterial blight disease. TLS=time taken for the lesions to entirely cover the leaf disc. SE (slope)=0.1228.

FIG. 7 is a graph depicting the relationship between the time taken for the lesions to entirely cover the leaf discs (TLS) in wet vs. dry season for 14 anthurium cultivars. TLS=time taken for the lesions to entirely cover the leaf disc.

FIG. 8 is a bar graph depicting the frequency distribution of TLS (days) for 50 anthurium cultivars evaluated for resistance to foliar bacterial blight disease in the wet season. TLS=time taken for the lesions to entirely cover the leaf disc.

FIG. 9 is a graph depicting the identification of anthurium cultivars that are resistant/tolerant to both systemic (time to death) and foliar infection by bacterial blight disease. Cultivars with no plants that died were given a time to death of 30 weeks. 1=‘Acropolis’; 2=‘Bianco’; 3=‘Venus’; 4=‘Local Pink’; 5=‘Cotopaxi’; 6=‘Mirjam’; 7=‘Tropical’; 8=‘Farao’; 9=‘Senator’; 10=‘Honduras’; 11=‘Spirit’; and 12=‘Local Mina Pink’.

FIG. 10 is a graph depicting the identification of anthurium cultivars that are resistant/highly tolerant (≦0.44) to systemic (proportion of dead plants) and foliar infection by bacterial blight disease. 1=‘Acropolis’; 2=‘Bianco’; 3=‘Spirit’; 4=‘Honduras’; 5=‘Terra’; 6=‘Local Pink; 7=‘Cotopaxi’; 8=‘Mirjam’; 9=‘Tropical’; 10=‘Farao’; 11=‘Venus’; 12=‘Senator’; and 13=‘Local Whitish Pink’.

FIG. 11A is a graph depicting the mean 5-day transpiration (ml) over all Anthurium andraeanum (Hort.) cultivars (diamonds) and mean 5-day transpiration (ml) for the individual cultivar (squares) Mirjam evaluated in vase-life Experiment 1 (dry season).

FIG. 11B is a graph depicting the mean 5-day transpiration (ml) over all Anthurium andraeanum (Hort.) cultivars (diamonds) and mean 5-day transpiration (ml) for the individual cultivar (squares) Venus evaluated in vase-life Experiment 1 (dry season).

FIG. 11C is a graph depicting the mean 5-day transpiration (ml) over all Anthurium andraeanum (Hort.) cultivars (diamonds) and mean 5-day transpiration (ml) for the individual cultivar (squares) Spirit evaluated in vase-life Experiment 1 (dry season).

FIG. 11D is a graph depicting the mean 5-day transpiration (ml) over all Anthurium andraeanum (Hort.) cultivars (diamonds) and mean 5-day transpiration (ml) for the individual cultivar (squares) Pierrot evaluated in vase-life Experiment 1 (dry season).

FIG. 11E is a graph depicting the mean 5-day transpiration (ml) over all Anthurium andraeanum (Hort.) cultivars (diamonds) and mean 5-day transpiration (ml) for the individual cultivar (squares) Fla Range evaluated in vase-life Experiment 1 (dry season).

FIG. 11F is a graph depicting the mean 5-day transpiration (ml) over all Anthurium andraeanum (Hort.) cultivars (diamonds) and mean 5-day transpiration (ml) for the individual cultivar (squares) Midori evaluated in vase-life Experiment 1 (dry season).

FIG. 11G is a graph depicting the mean 5-day transpiration (ml) over all Anthurium andraeanum (Hort.) cultivars (diamonds) and mean 5-day transpiration (ml) for individual cultivar (squares) for the cultivar Terra evaluated in vase-life Experiment 1 (dry season).

FIG. 11H is a graph depicting the mean 5-day transpiration (ml) over all Anthurium andraeanum (Hort.) cultivars (diamonds) and mean 5-day transpiration (ml) for individual cultivar (squares) for the cultivar Cuba evaluated in vase-life Experiment 1 (dry season).

FIG. 11I is a graph depicting the mean 5-day transpiration (ml) over all Anthurium andraeanum (Hort.) cultivars (diamonds) and mean 5-day transpiration (ml) for individual cultivar (squares) for the cultivar Tropical evaluated in vase-life Experiment 1 (dry season).

FIG. 11J is a graph depicting the mean 5-day transpiration (ml) over all Anthurium andraeanum (Hort.) cultivars (diamonds) and mean 5-day transpiration (ml) for individual cultivar (squares) for the cultivar Honduras evaluated in vase-life Experiment 1 (dry season).

FIG. 11K is a graph depicting the mean 5-day transpiration (ml) over all Anthurium andraeanum (Hort.) cultivars (diamonds) and mean 5-day transpiration (ml) for individual cultivar (squares) for the cultivar Lydia evaluated in vase-life Experiment 1 (dry season).

FIG. 11L is a graph depicting the mean 5-day transpiration (ml) over all Anthurium andraeanum (Hort.) cultivars (diamonds) and mean 5-day transpiration (ml) for individual cultivar (squares) for the cultivar Evergreen evaluated in vase-life Experiment 1 (dry season).

FIG. 11M is a graph depicting the mean 5-day transpiration (ml) over all Anthurium andraeanum (Hort.) cultivars (diamonds) and mean 5-day transpiration (ml) for individual cultivar (squares) Success evaluated in vase-life Experiment 1 (dry season).

FIG. 11N is a graph depicting the mean 5-day transpiration (ml) over all Anthurium andraeanum (Hort.) cultivars (diamonds) and mean 5-day transpiration (ml) for individual cultivar (squares) Tequila evaluated in vase-life Experiment 1 (dry season).

FIG. 11O is a graph depicting the mean 5-day transpiration (ml) over all Anthurium andraeanum (Hort.) cultivars (diamonds) and mean 5-day transpiration (ml) for individual cultivar (squares) Acropolis evaluated in vase-life Experiment 1 (dry season).

FIG. 11P is a graph depicting the mean 5-day transpiration (ml) over all Anthurium andraeanum (Hort.) cultivars (diamonds) and mean 5-day transpiration (ml) for individual cultivar (squares) Fantasia evaluated in vase-life Experiment 1 (dry season).

FIG. 11Q is a graph depicting the mean 5-day transpiration (ml) over all Anthurium andraeanum (Hort.) cultivars (diamonds) and mean 5-day transpiration (ml) for individual cultivar (squares) Local Pink evaluated in vase-life Experiment 1 (dry season).

FIG. 12 is a graph depicting the relationship between spadix and spathe necrosis in Anthurium andraeanum (Hort.) cultivars based on Experiment 1 (dry season).

FIG. 13 is a graph depicting the relationship between spadix necrosis and vase-life in 17 Anthurium cultivars evaluated for vase-life in Experiment 1 (dry season).

FIG. 14 is a graph depicting the correlation coefficients between average daily transpiration rates of cut-flowers of 17 anthurium cultivars measured at 5, 10, 15, 20, 25, 30, 35, 40, and 45 days and vase-life.

FIG. 15A is a scatter plot of 5-day transpiration means (diamonds) for Acropolis and mean 5-day transpiration for 16 Anthurium andraeanum (Hort.) cultivars (squares) evaluated for vase-life in Experiment 2, the wet season.

FIG. 15B is a scatter plot of 5-day transpiration means (diamonds) for Cuba and mean 5-day transpiration for 16 Anthurium andraeanum (Hort.) cultivars (squares) evaluated for vase-life in Experiment 2, the wet season.

FIG. 15C is a scatter plot of 5-day transpiration means (diamonds) for Evergreen and mean 5-day transpiration for 16 Anthurium andraeanum (Hort.) cultivars (squares) evaluated for vase-life in Experiment 2, the wet season.

FIG. 15D is a scatter plot of 5-day transpiration means (diamonds) for Fantasia and mean 5-day transpiration for 16 Anthurium andraeanum (Hort.) cultivars (squares) evaluated for vase-life in Experiment 2, the wet season.

FIG. 15E is a scatter plot of 5-day transpiration means (diamonds) for Fla Range and mean 5-day transpiration for 16 Anthurium andraeanum (Hort.) cultivars (squares) evaluated for vase-life in Experiment 2, the wet season.

FIG. 15F is a scatter plot of 5-day transpiration means (diamonds) for Honduras and mean 5-day transpiration for 16 Anthurium andraeanum (Hort.) cultivars (squares) evaluated for vase-life in Experiment 2, the wet season.

FIG. 15G is a scatter plot of 5-day transpiration means (diamonds) for Local Pink and mean 5-day transpiration for 16 Anthurium andraeanum (Hort.) cultivars (squares) evaluated for vase-life in Experiment 2, the wet season.

FIG. 15H is a scatter plot of 5-day transpiration means (diamonds) for Lydia and mean 5-day transpiration for 16 Anthurium andraeanum (Hort.) cultivars (squares) evaluated for vase-life in Experiment 2, the wet season.

FIG. 15I is a scatter plot of 5-day transpiration means (diamonds) for Midori and mean 5-day transpiration for 16 Anthurium andraeanum (Hort.) cultivars (squares) evaluated for vase-life in Experiment 2, the wet season.

FIG. 15J is a scatter plot of 5-day transpiration means (diamonds) for Mirjam and mean 5-day transpiration for 16 Anthurium andraeanum (Hort.) cultivars (squares) evaluated for vase-life in Experiment 2, the wet season.

FIG. 15K is a scatter plot of 5-day transpiration means (diamonds) for Pierrot and mean 5-day transpiration for 16 Anthurium andraeanum (Hort.) cultivars (squares) evaluated for vase-life in Experiment 2, the wet season.

FIG. 15L is a scatter plot of 5-day transpiration means (diamonds) for Success and mean 5-day transpiration for 16 Anthurium andraeanum (Hort.) cultivars (squares) evaluated for vase-life in Experiment 2, the wet season.

FIG. 15M is a scatter plot of 5-day transpiration means (diamonds) for Tequila and mean 5-day transpiration for 16 Anthurium andraeanum (Hort.) cultivars (squares) evaluated for vase-life in Experiment 2, the wet season.

FIG. 15N is a scatter plot of 5-day transpiration means (diamonds) for Terra and mean 5-day transpiration for 16 Anthurium andraeanum (Hort.) cultivars (squares) evaluated for vase-life in Experiment 2, the wet season.

FIG. 15O is a scatter plot of 5-day transpiration means (diamonds) for Tropical and mean 5-day transpiration for 16 Anthurium andraeanum (Hort.) cultivars (squares) evaluated for vase-life in Experiment 2, the wet season.

FIG. 15P is a scatter plot of 5-day transpiration means (diamonds) for Venus and mean 5-day transpiration for 16 Anthurium andraeanum (Hort.) cultivars (squares) evaluated for vase-life in Experiment 2, the wet season.

FIG. 16 is a graph depicting the relationship between average stomata density across seasons and stomata densities in the dry and wet seasons for 16 Anthurium andraeanum (Hort.) cultivars. Ab. Stom.=Abaxial stomata density. Diamonds=Ab. Stom. (dry season), squares=Ab. Stom. (wet season), triangles=Ab. Stom. (expected, dry season), and crosses=Ab. Stom. (expected, wet season).

FIG. 17 is a graph depicting the relationship between average vase-life across seasons and vase-lives in the dry and wet seasons for 16 Anthurium andraeanum (Hort.) cultivars. Diamonds=vase-life (dry season), squares=vase-life (wet season), triangles=vase-life (expected, dry season), and crosses=vase-life (expected, wet season).

FIG. 18 is a graph depicting the relationship between average spadix necrosis across seasons and spadix necrosis in the dry and wet seasons for 16 Anthurium andraeanum (Hort.) cultivars. Diamonds=spadix necrosis (dry season), squares=spadix necrosis (wet season), triangles=spadix necrosis (expected, dry season), and crosses=spadix necrosis (expected, wet season).

FIG. 19 is a graph depicting the relationship between average spathe necrosis across seasons and spathe necrosis in the dry and wet seasons for 15 Anthurium andraeanum (Hort.) cultivars. Diamonds=spathe necrosis (dry season), squares=spathe necrosis (wet season). Significant genotype×season interactions at P≦0.001.

FIG. 20 is a graph depicting dry vs. wet season abaxial stomata density of 16 anthurium cultivars.

FIG. 21 is a graph depicting dry vs. wet season vase-life of 16 anthurium cultivars.

FIG. 22 is a graph depicting the relationship between actual and expected vase-life for 16 Anthurium andraeanum (Hort.) cultivars evaluated in the second vase-life experiment (wet season). SE (slope)=0.1266.

FIG. 23 is a graph depicting the relationship between actual and expected time to spadix necrosis for 16 Anthurium andraeanum (Hort.) cultivars evaluated in the second vase-life experiment (wet season). SE (slope)=0.1734.

FIG. 24 is a graph depicting the relationship between actual and expected vase-life for the 9 Anthurium andraeanum (Hort.) cultivars evaluated in vase-life Experiment 3 using the vase-life regression equation developed in vase-life Experiment 1. SE (slope)=0.2533.

FIG. 25 is a graph depicting the relationship between actual and expected time to spadix necrosis for the 9 Anthurium andraeanum (Hort.) cultivars evaluated in Experiment 3 using the spadix necrosis regression equation developed in vase-life Experiment 1. SE (slope)=0.1901.

FIG. 26 is a diagram illustrating the phenylpropanoid pathway leading the various colored anthocyanin compounds (after Collette, 2002).

FIG. 27 is a diagram illustrating a genetic model for spathe color in anthurium based on a model with genes M, O and R.

### DETAILED DESCRIPTION

Anthurium andraeanum (Hort.) is an important export ornamental crop in the Caribbean. The anthurium industry, during the past decade, has been debilitated by the bacterial blight disease caused by Xanthomonas campestris pv. dieffenbachiae. Progress in breeding for resistance to the bacterial blight disease has been hampered globally, due to lack of suitable screening methods and a poor understanding of the genetics of resistance. An optimized petiole inoculation method using a fluorescent strain of X. campestris pv. dieffenbachiae was used to identify genotypes with systemic resistance to disease organisms. A leaf-disc inoculation method was developed to quantitatively assess foliar resistance. Studies using these methods demonstrated that systemic and foliar resistances were under different genetic control. Two major dominant genes, interacting in a duplicate recessive epistasis manner, were responsible for systemic resistance, while polygenic inheritance with a predominance of additive genetic effects accounted for foliar resistance. The narrow sense heritability estimates for systemic and foliar resistance were 42.5% and 92%, respectively.

Provided herein are optimized screening methods for assessing foliar and systemic resistance to bacterial blight disease. The method reduces the time required for identification of resistant cultivars, for example, from about 28 weeks to about 5 weeks.

Screening methods are also provided for vase-life and other horticultural characteristics. A study of vase-life of anthurium revealed that senescence in anthurium cut-flowers was induced by water stress, and can be measured as the time to spadix necrosis. Vase-life was influenced by spathe color and abaxial stomatal density, and can be predicted using the following equation: ‘vase-life (days)=29.1−1.99 (abaxial stomatal density)+18.3 (green/not green)+18.5 (white/not white)’.

The methods provided herein can be used to identify cultivars suitable for commercial purposes and for identification of parental cultivars useful for developing novel cultivars that have relatively high resistance to bacterial blight disease and/or increased vase-life, for example, the methods can be used as part of a breeding program . . . .

### Screening for Resistance to Xanthomonas

Bacterial blight disease caused by Xanthomonas, e.g., Xanthomonas campestris pv. dieffenbachiae (=Xanthomonas axonopodis pv. dieffenbachiae) (Vauterin et al, 1995, Intl. J. Systematic Bacteriology 45:472-489) affects a broad range of ornamental and edible aroids. Methods are provided herein for screening plants for resistance to infection by a Xanthomonas.

The plant genus, species, or cultivar that is being tested is referred to herein as a “target plant.” A method has been developed for screening a plant species for resistance to Xanthomonas strains that can infect the plant species. The method is demonstrated herein using an optimized petiole inoculation method using a fluorescent strain of X. campestris pv. dieffenbachiae that can be used to identify genotypes of Anthurium with systemic resistance. In addition, a leaf-disc inoculation method has been developed that can be used to quantitatively assess foliar resistance.

Plants Affected by Xanthomonas and Suitable for Screening

The method of screening for Xanthomonas resistance that is disclosed herein can be used for any genus, species, or cultivar that is susceptible to infection with a Xanthomonas. The genera that can be tested include, for example, Anthurium, Philodendron, Dieffenbachia, Colocasia (taro), Aglaonema, Syngonium, Xanthosoma, Epipremnum, Dracaena, Alocasia, Spathi phyllum, Raphidophora, and Caladium.

Xanthomonas for Screening Assays

Species of Xanthomonas that can infect a target plant are used in the screening methods described herein. Suitable strains of the Xanthomonas species can be obtained from depositories or can be isolated from field samples, e.g., isolated from infected plants or from soil.

Methods of isolating Xanthomonas from field samples are known in the art, for example Lipp et al. (1992) and Fukui et al. (1998). In general, a Xanthomonas strain isolated from a field sample is tested for pathogenicity by infecting healthy plants or plant tissues, for example, using the method described in Fukui et al. (1998, supra). Pathogenicity is determined using methods known in the art, such as lesion size after incubation the infected plant or tissue for a selected amount of time.

Recombinant Xanthomonas

Strains of Xanthomonas that are suitable for use in the screening method provided herein are capable of infecting the target plant (e.g., an Anthurium), and stably express a heterologous fluorescent protein such as Green Fluorescent Protein. In general, the fluorescence resulting from the heterologous protein is distinguishable from any autofluorescence endogenous to the Xanthomonas strain. A Xanthomonas strain having the required properties is referred to herein as a “fluorescent Xanthomonas.”

A recombinant Xanthomonas strain is generated by introducing a gene expressing a heterologous fluorescent protein is introduced into a Xanthomonas strain that can infect a target plant. Methods for introducing a heterologous gene into bacteria, including Xanthomonas are known in the art. For example, a gene encoding a fluorescent protein is introduced into a plasmid vector comprising a promoter (an inducible promoter or a constitutive promoter) suitable for expression in a Xanthomonas such as a lac promoter or npt-2 promoter. Typically, a broad host range vector is suitable for use although a Xanthomonas-specific vector may be used. Examples of suitable plasmids and methods of making such plasmids are known in the art, for example, p519 ngfp (Matthysse et al., 1996, FEMS Microbiol. Lett. 145:87-89).

The gene expressing the fluorescent protein can encode, for example, a Green Fluorescent Protein (GFP), a Blue Fluorescent Protein (BFP), a Yellow Fluorescent Protein (YFP), a cyan fluorescent protein (CFP) or variants thereof. Sequences encoding such proteins are known in the art. In some cases, the nucleic acid sequence encoding the protein has been adapted for expression in a prokaryote.

A heterologous gene encoding a fluorescent protein is introduced into a Xanthomonas strain using any suitable method known in the art. For example, electroporation or calcium shock can be used to introduce a nucleic acid encoding a fluorescent protein into the selected Xanthomonas strain. After transformation, bacteria are selected for stable expression of the fluorescent protein. The nucleic acid that stably expresses the fluorescent protein can be extrachromosomal or integrated (e.g., using a method such as that of Kalogeraki et al. (1997, Gene 188:69-75) employing a suicide plasmid) into the bacterial chromosome. Bacterial strains that stably express a heterologous fluorescent protein are termed herein “fluorescent Xanthomonas.” In some cases, a fluorescent Xanthomonas strain is tested for its ability to infect a target plant compared to the parent strain, e.g., to ensure that infectivity was not significantly affected by expression of the fluorescent protein.

Screening for Systemic Resistance to Xanthomonas Infection

To screen a target plant for resistance to Xanthomonas infection, fluorescent Xanthomonas are cultured and diluted to selected concentrations (e.g., CFU/ml as determined by comparison of culture density to a reference calibration curve). Target plants are inoculated with the fluorescent Xanthomonas. In some cases, the plants are inoculated using the method of Fukui et al. (1998, supra), which is performed by selecting the second youngest leaf, cutting the petiole 2.5 cm from the stem base, and injecting 100 μl of inoculum. The amount of inoculum used can be determined empirically. In general, an inoculum concentration equivalent to about 10⁹ CFU/ml is used. In some cases, about 10⁷ CFU/ml or about 10⁸ CFU/ml is used.

Infected cultivars are then grown and evaluated, e.g., once per week, after two weeks, after three weeks, after four weeks, after fives, or after at least five weeks, for fluorescence associated with the fluorescent protein expressed by the fluorescent Xanthomonas in a plant tissue other than the inoculated leaf. For example, a section of the petiole, midrib, or lamina of a leaf immediately adjacent to the inoculated leaf is macerated in distilled water or buffer, incubated for a selected time (e.g., 15 minutes, 30 minutes, one hour), the sample is cleared of debris, and the presence of the fluorescent protein is assayed in the supernatant.

The presence of fluorescence indicates propagation of Xanthomonas and infection by the Xanthomonas. The lack of fluorescence in the assayed tissue after a suitable incubation period (e.g., two weeks, three weeks, four weeks, five weeks, or at least five weeks) indicates resistance to the Xanthomonas. A minimum incubation period can be determined by selecting the minimum amount of time needed for a cultivar of the target plant known to be susceptible to infection to demonstrate propagated fluorescence.

In some screening methods, more than one plant of a cultivar is infected. If no plants of the cultivar demonstrate propagation of Xanthomonas after a selected incubation period, the cultivar is designated resistant to Xanthomonas. If some, but not all plants demonstrate propagation, the cultivar is designated to be of intermediate resistance. Cultivars that demonstrate 100% propagation are designated susceptible to Xanthomonas infection. Resistant cultivars are desirable for cultivation in regions in which Xanthomonas is present. In some cases, cultivars of intermediate resistance are useful for propagation in such regions. Furthermore, resistant cultivars or intermediate resistance cultivars are useful for breeding programs, e.g., to establish new cultivars that have desirable characteristics in addition to Xanthomonas resistance.

Screening for Foliar Resistance

Blight has two phases, a foliar phase and a systemic phase. In the foliar phase, the pathogen invades the foliage of the plant. In susceptible cultivars, a foliar infection can rapidly progress into a systemic phase resulting in plant death. Anthurium cultivars demonstrate differential susceptibility to foliar and systemic phases of blight (Fukui et al., 1998, supra). A method of screening plants for foliar resistance to Xanthomonas was also developed. Briefly, the method involves an inoculation method that uses vacuum infiltration of leaf discs with a Xanthomonas. Vacuum infiltration is typically carried out by immersing a leaf disc in a Xanthomonas inoculum at a known density (e.g., 10⁷ CFU/ml, 10⁸ CFU/ml, 10⁹ CFU/ml) and applying vacuum of specific magnitude and for a selected time (e.g., 15 psi, 5 seconds). Those in the art will understand how to adjust the conditions for vacuum infiltration to obtain effective inoculation.

After incubation for a suitable time (i.e., time sufficient for lesions to develop) leaf discs are examined for lesion size. In some methods, a leaf disc is examined for lesions several times over a period of days. In a non-limiting example, the leaf disc is examined at 6 days, 12 days, and 16 days. Those in the art will understand how to select other regimes. Parameters assayed can be, for example, time taken for an expanding lesion to cover the entire disc (TLC) and the lesion expansion rate (LER). Lesion size is measured using the leaf area meter, for example, by tracing the lesion area onto clear plastic and transcribing the area onto paper. The area of the cut-outs can be determined using a leaf area meter (ΔT Area Meter Mkt model, Delta-T Devices, England) and used as a covariate to correct final lesion size. Typically, the method of screening for foliar resistance employs TLC as a measure. More than one inoculum concentration can be used on separate samples to further differentiate the resistance to foliar infection. For example, and inoculum density of about 10⁷ CFU/ml, about 10⁸ CFU/ml, or about 10⁹ CFU/ml. In some cases, the measure of foliar resistance is lesion size at a selected day after inoculation, e.g., day 6.

Cultivars having relatively high resistance to foliar infection by Xanthomonas are useful for, e.g., breeding programs to develop cultivars that are resistant to or have improved resistance to foliar infection compared to related species or cultivars of a species. In some cases, the screened species or cultivars are of the genus Anthurium.

Screening for Systemic and Foliar Resistance to Xanthomonas

It was reported that some cultivars resistant to foliar bacterial blight disease succumb rapidly to systemic infection and vice versa (Prior et al., 1985, supra; Fukui et al., 1996; Fukui et al., 1998, supra; and Fukui et al, 1999(a), Phytopathology, 89:1007-1014), indicating a two-phase differential basis for the disease.

Using the developed screening methods, it was discovered that systemic and foliar resistances of Anthurium were under different genetic control. Two major dominant genes, interacting in a duplicate recessive epistasis manner, were found to be responsible for systemic resistance, while polygenic inheritance with a predominance of additive genetic effects accounted for foliar resistance. The narrow sense heritability estimates for systemic and foliar resistance were 42.5% and 92%, respectively. Further experiments comparing systemic resistance and foliar resistance demonstrated poor correlation with foliar resistance, which indicates that systemic and foliar resistance are regulated by different mechanisms.

Accordingly, methods provided herein include screening of cultivars for both systemic resistance and foliar resistance, which enables identification and propagation of cultivars with resistance to Xanthomonas at both levels of infection. In such methods for assaying systemic resistance and foliar resistance, any combination of the methods described herein for separately assaying each feature can be used.

Additional Features of Xanthomonas Resistance Assays

Multiplication of pathogenic Xanthomonas in the leaf tissues of susceptible cultivars is controlled by temperature (Alvarez et al, 1991, Proc. Fourth Hawaii Anthurium Indus. Conf., pp 12-18), and unrestricted by host defense systems (Fukui et al., 1999, supra). Resistant cultivars are generally less sensitive to temperature changes (Fukui et al., 1999a, supra). Accordingly, the assays for Xanthomonas resistance are typically conducted at controlled temperatures. In some cases, resistance at various temperatures is assayed to determine the range of temperatures at which a cultivar is resistant.

Fukui et al. (1999a, supra) indicated that host defense mechanisms activated by disease resistance genes restrict multiplication of the pathogen only after symptom expression or after the bacterial population reaches 10⁴ CFU/mm² in leaf tissues. It was further suggested that the defense mechanism for bacterial blight disease resistance overshadows temperature effects and restricts multiplication of the pathogen in the leaf tissues of resistant cultivars (Fukui et al., 1999a). Typically, sufficiently high densities of pathogen are used in an assay to assure routine establishment of infection and avoid escapees.

Using the method described herein, the genetics of resistance to the Xanthomonas has been elucidated. This allows for the first time the ability to genotype individual accessions and thereby produce anthurium varieties through breeding that do not harbor susceptible alleles. This is important because it results in genotypes that cannot harbor the Xanthomonas without showing symptoms. This is the first time a method for breeding for complete resistance has been developed for anthurium.

Selection for Vase-life of Anthurium

Anthurium cut-flowers are known for their relatively long vase-life that ranges from a week to several months. Anthurium importers prefer cultivars with a long vase-life to ensure that the cut-flowers will retain their freshness throughout the transportation, distribution and retail chain. Commercially, a vase-life of three weeks and over (Kamemoto and Kuehnle, 1996, Breeding Anthuriums in Hawaii, Honolulu, Hi., U. Hawaii Press) is required to market anthurium cut-flowers.

Halevy and Mayak (Halevy and Mayak, 1979, Hort. Rev., 1:204-236) defined vase-life as the useful longevity of the floral product at the final consumer's home. There is no agreed set of criteria for measuring vase-life in anthurium; as what is deemed unsaleable by a wholesaler or retailer is different to what a consumer is prepared to discard (Paull, 1982, HortSci. 17(4):606-607). Shirakawa et al. (Shirakawa et al, 1964, Proc. Am. Soc. Hort. Sci., 85:642-646) used spathe wilting, spathe or spadix darkening as indications of vase-life termination, while Paull (1982, supra) regarded loss of spathe glossiness and spathe blueing as well. Hence, usually complex criteria are used to measure vase-life in anthurium, in which vase-life is determined by whichever of the above symptoms appears first.

Although vase-life is considered an important horticultural quality character in anthurium, there have been few studies conducted to determine the characteristics associated with variation in vase-life. Although post-harvest studies have pointed to dehydration and protein breakdown as possible mechanisms, this has not led to any practical selection criteria for improving vase-life in plant breeding programs. In addition, most studies in the past have used only a few cultivars.

The present invention relates to methods for assessing morpho-physiological characteristics of the anthurium cut-flower, determining their relationship to vase-life; and a method for determining vase-life based on the morpho-physiological characters evaluated.

Prediction of Vase-Life Based on Spadix Necrosis

As described herein, studies were conducted to determine the relationship between various indicators of vase-life in anthurium. The studies demonstrated that time to spadix necrosis is the best measure of vase-life in anthurium cut-flowers for several reasons: Spadix necrosis is the earliest occurring senescence symptom that is common to all anthurium cultivars, has the best differentiation index, and is highly correlated to all other important senescence symptoms. Furthermore, the studies demonstrated that vase-life determined by more complex criteria than simply spadix necrosis correlated well with vase-life as determined using only spadix necrosis as a measure, and has a similar error estimate. Accordingly, methods are provided herein for evaluating vase-life by evaluating spadix necrosis, methods for which are known in the art. The precise number of cut-flowers to be evaluated and conditions under which the flowers are handled can be determined by one skilled in the art, typically, the selected conditions are similar to those under which cut-flowers used for commercial purposes are handled. In one example, the assay for vase-life is determined by observing the first sign of spadix necrosis in two out of the three cut-flowers contained in each experimental unit. The assay for vase-life based on spadix necrosis can be used to select cultivars, e.g., for breeding programs or commercial cultivation. Typically, a cultivar selected as having a vase-life suitable for commercial purposes demonstrates a vase-life of at least three weeks, e.g., at least four weeks, at least five weeks, or at least six weeks, under conditions used for commercial transportation, distribution, and sale.

Prediction of Vase-Life Based on Density of Abaxial Stomata

It was found that the ability of an anthurium cultivar to maintain transpiration rates later in the life of the cut-flower determines its vase-life. The correlation between abaxial stomata density and transpiration was significant as was the correlation between spathe color. Accordingly, a method for predicting vase-life was developed that is based on density of abaxial stomata and spathe color.

In general the method of predicting vase-life is based on the finding that vase-life is influenced by spathe color and abaxial stomatal density, and that vase-life can be predicted using the following equation: vase-life (days)=29.1−1.99 (abaxial stomatal density)+18.3 (green/not green)+18.5 (white/not white). In some cases, the predicted vase-life is compared to a reference, for example a set of correlations for Anthurium cultivars having known information for vase-life, abaxial stomatal density, and spathe color. In general, plants (e.g., cultivars) having a predicted vase-life of at least three weeks, e.g., at least four weeks, at least five weeks, or at least six weeks are useful for breeding programs or commercial purposes.

Both the density of abaxial stomata and spathe color can be determined using methods known to those in the art, and using methods described in the Examples. An alternative way of determining the stomatal density is to apply a coat of nail varnish, allow it to dry, peel off the nail varnish, and count the number of stomatal impressions under a light microscope.

The method provides the means to breed for improved vase-life in anthuriums, as conventional vase-life experiments are time consuming, economically not feasible and difficult to conduct in segregating populations (without replications).

### EXAMPLES

The invention is further illustrated by the following examples. The examples are provided for illustrative purposes only. They are not to be construed as limiting the scope or content of the invention in any way.

### Example 1

### Screening For Resistance to Xanthomonas Campestris pv. Dieffenbachiae in Anthurium

### Use of Bacterial Blight Resistant Anthurium Clones

The most effective control measure for bacterial blight disease is the use of resistant cultivars (Prior et al, 1985, Agronomie Tropicale 42:61-68; Anïs et al, 2000, Acta-Horticulture 408:135-140). The use of such cultivars will reduce the cost and risk associated with anthurium cultivation.

Conventional breeding methods require a screening method that can discriminate between susceptible and resistant plants. Susceptible, non-symptomatic plants cannot be easily discerned from resistant plants, and require laborious and expensive methods such as back indexing, use of selective media or serological techniques.

The use of a bioluminescent strain of X. campestris pv. dieffenbachiae developed by Fukui et al. (1998, supra) provided an effective method of screening resistance in large segregating populations of anthurium but was costly and laborious, requiring exposure of leaves to X-ray film.

As provided herein, the use of a Xanthomonas strain engineered to express a gfp gene simplifies the detection of the pathogen within non-symptomatic tissues and organs. The screening method can also be used to study the mechanism of resistance to bacterial blight disease at both foliar and systemic levels and its genetic basis in the anthurium-X. campestris pv. dieffenbachiae, host-pathogen system.

Accordingly, the present invention relates to an improved method for screening cultivars of susceptible genera (e.g., anthurium cultivars) for resistance to bacterial blight disease caused by Xanthomonas (e.g., Xanthomonas campestris pv. Dieffenbachiae) at the foliar and systemic levels, thereby identifying cultivars suitable for commercial purposes or suitable for use in breeding programs.

### Material and Methods

Location

All screening experiments were carried out in shade-houses on a commercial anthurium farm, Kairi Blooms Ltd. The screen-houses were located well away from the commercial shade-houses, with a separate drainage system. A double layer of saran netting was placed all around the shade-house and covered with 75% shade saran. A clear plastic covering was also placed over the saran to prevent rain from splashing on plants and to maintain high humidity.

Cultivars Used in Screening Assays

Sixty cultivars were evaluated for resistance to systemic infection by bacterial blight disease caused by X. campestris pv. dieffenbachiae, and are presented in Table 1. Forty-nine cultivars were evaluated for resistance to foliar infection by bacterial blight disease (Table 1).

TABLE 1
Anthurium andraeanum (Hort.) cultivars evaluated for resistance to
systemic and foliar infection by bacterial blight disease.

| | | | Resistance |
|---|---|---|---|
| | | | ranking |
| | Experiment | | (Foliar |
| Cultivar | used | | infection)^{b} |
| | | Resistance | |
| | | ranking | |
| | | (Systemic | |
| | | infection) | |
| Acropolis | 2, 5, 6 | 1 | — |
| Alexis | 3, 5 | — | — |
| Andes 115 | 3, 5 | — | — |
| Avonette | 3 | — | — |
| Bianco | 3, 5 | — | — |
| Champagne | 2, 6 | 4 | — |
| Cheers | 2, 4 | 6 | 3 |
| Cotopaxi | 3, 5 | — | — |
| Cross 163 | 3 | — | — |
| Cross 2135 | 3, 5 | — | — |
| Cross 2306 | 5 | — | — |
| Cross 356 | 6 | — | — |
| Cross 3962 | 3, 5, 6 | — | — |
| Cross 4038 | 3 | — | — |
| Cross 4835 | 5 | — | — |
| Cross 793 | 3 | — | — |
| Cuba | 2, 4 | 7 | 6 |
| Cumbia | 3, 5 | — | — |
| Fantasia | 2, 4 | 7 | 8 |
| Farao | 3, 5 | — | — |
| Fla Range | 3 | — | — |
| Furore | 3, 5 | — | — |
| Gloria | 2, 4 | 5 | 7 |
| Hawaii | 2, 4 | 7 | 2 |
| Honduras | 2, 5, 6 | 3 | — |
| Ibara | 2, 5, 6 | 1 | — |
| Kalapana | 2, 5 | 3 | — |
| Laguna | 3, 5 | — | — |
| Lima | 3 | — | — |
| Linda | 3 | — | — |
| Local Cup White | 3 | — | — |
| | | Resistance | |
| | | ranking | |
| | | (Systemic | |
| | | infection)^{a} | |
| Local Mina Pink | 3, 5 | — | — |
| Local Mina Red | 3, 5 | — | — |
| Local Mina White | 3, 5 | — | — |
| Local Orange | 2, 4, 6 | 2 | — |
| Local Pink | 3, 5 | — | — |
| Local Whitish | 3, 6 | — | — |
| Pink | | | |
| Lunette | 3 | — | — |
| Lydia | 1, 2, 4, 6 | 3 | 4 |
| Margaretha | 3 | — | — |
| Midori | 2, 4 | 6 | 3 |
| Minuet | 3, 5 | — | — |
| Mirjam | 2, 4, 6 | 3 | 3 |
| Patricia | 3 | — | — |
| Pierrot | 2, 4, 6 | 5 | 2 |
| Pistache | 5 | — | — |
| President | 3, 5 | — | — |
| Rapido | 3, 5 | — | — |
| Rosa | 1, 2, 4 | 3 | 2 |
| Safari | 3, 5 | — | — |
| Senator | 2, 4 | 1 | 3 |
| Sonate | 2, 5 | 4 | — |
| Spirit | 2, 5 | 4 | — |
| Success | 1, 2, 5, 6 | 4 | — |
| Sweety | 3, 5 | — | — |
| Tequila | 3, 5 | — | — |
| Terra | 3, 5 | — | — |
| Tropical | 1, 2, 4 | 4 | 4 |
| Venus | 2, 4, 6 | 2 | 3 |
| Victoria | 3, 5, 6 | — | — |

Resistance to systemic infection by bacterial blight disease:
1 = Optimization of inoculum concentration;
2 = Repeatability of optimized screening method;
3 = Further screening of cultivars.
Resistance to foliar infection by bacterial blight disease:
4 = Optimization of inoculum concentration and repeatability of screening method in the wet season;
5 = Further set of cultivars evaluated in the wet season;
6 = Cultivars evaluated using optimized screening method in the wet and dry seasons.
^{a,b}Score 1 means most resistant, 10 means most susceptible at the systemic and foliar levels.
A ‘—’ indicates that the performance of the cultivars with regard to resistance bacterial blight disease was not available.

Cultivar performance (resistance to systemic and foliar infection by bacterial blight disease) under field conditions was obtained through a survey of local farms in Trinidad in 1999 by Avey. Farmers were asked to score on a scale of one to ten, one being the most resistant and ten being the most susceptible. Scores for each cultivar were totaled and the mean performance of the cultivar at the systemic and foliar levels was calculated to the nearest natural number (1-10).

Isolation of X. Campestris Pv. Dieffenbachiae and Koch's Postulate

Diseased leaves were obtained from systemically infected anthurium plants showing typical symptoms of X. campestris pv. dieffenbachiae during the wet season of 2001 from a commercial anthurium farm (Kairi Blooms Farms Ltd). The leaves were placed in labeled polythene bags and transported to the laboratory. The leaves were surface sterilized with 70% ethanol, and small leaf pieces dissected from the advancing margins of the lesion were macerated in sterile distilled water. A loop full of the water from the macerated tissues was streaked onto freshly prepared Luria agar (Miller's modification, Sigma) plates under a laminar flow hood, and incubated at 30° C. in an incubator (Gallenkamp, UK; cat. no. 1NA-300-130M). After 48-72 hours, single, mucoid, yellow bacterial colonies were carefully collected using a sterile inoculation loop and sub-cultured onto Luria agar plates to obtain pure cultures, which were confirmed as X. campestris pv. dieffenbachiae.

Ten colonies from each of ten pure cultures were randomly selected, plated onto freshly made Luria agar slants and allowed to grow for 48 hours. The bacteria were washed with sterile distilled water and diluted to 50% transmittance using the 600 nm wavelength of a GENESYS 20 spectrophotometer (Model: 4001/4, Thermo Spectronic, USA).

Koch's postulates was tested by injecting 0.1 ml (100 μl) of the diluted inoculum into the lamina of leaves of a susceptible anthurium cultivar, ‘Fantasia’, using a B-D ultra fine^{(MR)} 1.0 ml syringe (Becton Dickinson, Mexico). Each pure culture was inoculated separately into two leaves. A leaf of the same cultivar was inoculated with 0.1 ml sterile distilled water and used as a negative control. Leaves were of similar age (stage 2; as defined by Fukui et al., 1998) and size. Following inoculation, each leaf was placed adaxial surface up inside a transparent plastic bag containing moist tissue paper and incubated for 20 days at ambient temperature (30° C.). The experiment was arranged in a completely randomized design with two replications. Symptoms were noted daily and the isolates were scored according to their aggressiveness based on lesion size. The isolate with the largest expanding lesion at 20 days after inoculation (DI) was considered the most aggressive. The aggressive isolates from Koch's postulates were re-cultured on Luria agar and maintained on YDC slants (Jones and Chun, 2001, Laboratory Guide for Identification of Plant Pathogenic Bacteria, 3d ed. Minnesota, USA: APS Press) as stock cultures. The most aggressive isolate (X₄) was used in all subsequent experiments.

Transformation of X. Campestris Pv. Dieffenbachiae with p519 ngfp

Determining the Mid-Log Growth Phase of X. Campestris Pv. Dieffenbachiae

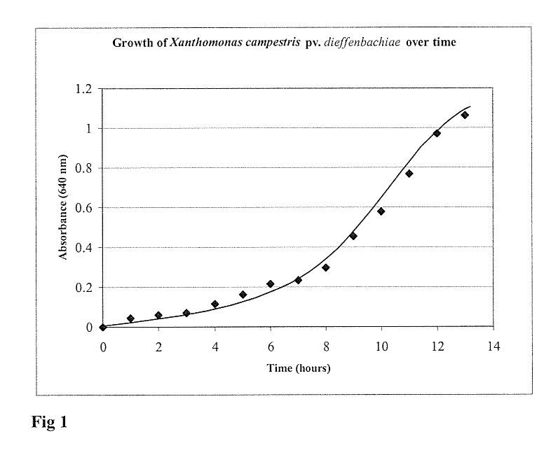

Isolate X₄ of X. campestris pv. dieffenbachiae was plated onto two freshly prepared Luria agar plates (one loopful per plate), and incubated for 48 hours at 30° C. (Incubator size 2, cat no.: 1NA-300-130M, Gallenkamp, UK). One loopful of bacteria was taken from each plate and placed respectively in 250 ml bottles (Nalgene®) containing 100 ml of Luria broth (GIBCO BRL). The bottles were capped to allow for gaseous exchange and incubated at 30° C. in a shaking incubator (serial no: SG94/12/188, Gallenkamp, UK) at 225 rpm. One-ml aliquots were pipetted hourly and the optical density measured at 640 nm using the ‘time drive program’ of a spectrophotometer, (Perkin Elmer UV/VIS Spectrometer Lambda 11, ID No.: BL111209, Germany) until there was no further change in OD (plateau phase). The mean OD values for each hour were used to plot a growth curve for X. campestris pv. dieffenbachiae and the mid-log phase was determined to coincide at OD 0.6 (640 nm) (FIG. 1).

Preparation of Competent Cells for Transformation

A loopful of freshly cultured (48 hours old) bacteria on Luria agar was placed in a Nalgene® 250 ml bottle containing 100 ml of Luria broth (GIBCO BRL). The bacteria were grown to mid-log phase (about OD 0.6 at 640 nm). Bacterial cells were aliquoted into four sterile, ice-cold 50 ml polypropylene tubes (cat. no. 03535, Sorvall, Newton, Conn., USA), placed on ice for ten minutes and then centrifuged at 5150×g for ten minutes at 4° C.

The supernatant was discarded and the cells in each tube were washed with 1 ml cold sterile deionized water. Cells were resuspended in 10 ml of cold 10% glycerol and centrifuged at 5150×g for ten minutes at 4° C. (Sorvall® RC-285, Dupont, USA) and the supernatant discarded (White and Gonzalez, 1991, Phytopathology 81:521-524). Cells were resuspended in 1 ml cold sterile 10% glycerol and transferred to cold, sterile 1.5 ml microfuge tubes. The tubes were centrifuged at 5150×g for 1.5 minutes in a microfuge (Beckman Microfuge™12, USA) at room temperature and the supernatant discarded. The tubes were immediately placed on ice and 10% glycerol was added to each tube to a final volume of 0.5 ml and transformation was performed.

Plasmid Isolation

The E. coli strain, DH5a, containing the plasmid, p519 ngfp (size 9.2 kb; American Type Culture Collection; ATCC number, 87453) was plated onto Luria agar plates augmented with 50 mg/l kanamycin. Colonies were observed for fluorescence using a fluorescence microscope (Leica DM IRB, serial no: 090-132.707, Germany), and fluorescent colonies were plated onto new Luria agar plates containing 50 mg/l kanamycin.

Plasmid isolation was performed using the alkaline lysis procedure of Sambrook et al. (Sambrook et al, 1989) Molecular Cloning: A Laboratory Manual, 2d. ed., New York, USA: Cold Spring Harbor Laboratory Press). The following day, an RNAase digestion was performed and the purified plasmid DNA (10 μl) was electrophoresed on a 0.7% agarose gel containing 2 μl ethidium bromide (10 mg/ml). A high molecular weight marker (Invitrogen, cat. no. 15618-010, lot no. 1114004) was used as a molecular ladder.

Transformation Procedure

Three μl of plasmid DNA were placed in the bottom of an ice-cold sterile bacterial electroporation cuvette (50×1 mm ECU-101, Equibio). To this was added 50 μl of ice-cold competent bacterial cells. The cuvette was tapped lightly to ensure mixing of the bacterial cells and the plasmid DNA. Electroporation was performed immediately using the following parameters: 6 ms, 800 mFD and 450 V (X-CELL Electroporation System, Promega) Immediately following electroporation, 100 μl of YT-broth (Atlas, 1997) was added to the cells that were then kept at room temperature. Four independent electro-transformations were performed. The bacterial cells were then spread-plated onto pre-warmed (30° C.) freshly prepared Luria agar plates with and without 50 mg/l kanamycin.

Approximately 72 hours after plating, individual colonies from the plates (containing kanamycin) were picked and tested for successful transformation by viewing under the L63× lens of the Leica fluorescence microscope (fluorescence filter tube turret No. 3 and IC objective prism turret: BF DF PH FL). Bright green colonies, one from each of the four plates, were selected and maintained separately on fresh Luria agar plates and as stocks on YDC slants.

Koch's postulates were carried out using the transformed cells as described supra using stage-2 leaves of the cultivar, ‘Fantasia’ to identify virulent transformed colonies. Leaves were monitored daily for 13 days and the most aggressive isolate X_{4T2} was used in further work.

Aggressiveness of Transformed (X_{4T2}) Vs. Untransformed (X₄) X. Campestris Pv. Dieffenbachiae

The most aggressive isolates of transformed and untransformed bacteria were grown on Luria agar slants described above, washed with sterile distilled water and diluted to 13% transmittance (600 nm wavelength). Inoculation was performed as described above on stage 2 leaves of the cultivar, ‘Fantasia’ (transformed and untransformed bacteria). The experiment was carried out with five replications using appropriate negative controls as described above, in a completely randomized design. The lesion sizes elicited by the bacterial strains per leaf were traced at regular time intervals and lesion area determined using the leaf area meter (ΔT Area Meter MK2 model, Delta-T Devices, Burwell Cambridge, England).

A one-way ANOVA was carried out using the general linear model to test whether there were significant differences (α=0.05) between untransformed and transformed X. campestris pv. dieffenbachiae (NCSS statistical package). Comparison of regression lines (COLR program, Version 1, December 1974, Caribbean Agricultural Research and Development Institute, CARDI) was performed to determine whether the regression lines for the untransformed and transformed X. campestris pv. dieffenbachiae could be explained by a single regression line.

The mean lesion sizes over the study period were plotted for untransformed vs. transformed X. campestris pv. dieffenbachiae. A linear regression analysis was performed to determine the relationship between lesion expansions for transformed vs. untransformed isolates (NCSS statistical package) on host leaf tissue.

Optimizing the Screening Method for Systemic Infection of Anthurium Andraeanum (Hort.) by X. Campestris Pv. Dieffenbachiae

Optimizing the Inoculum Concentration

One loopful of bacteria each was streaked onto three 100 ml Luria agar slants made in 250 ml Nalgene® bottles and incubated at 30° C. Slants were washed and serial dilutions were made according to the “Dilution procedure for cuvettes” described by Benson (Benson, 1990, Microbial Applications, a Laboratory manual in General Microbiology, 5d ed. USA: Wm. C. Brown Publishers). After the sixth order dilution, following a 3 ml to 3 ml serial dilution, 10 ml serial dilutions were performed up to 10⁻⁷. For each of these dilutions the OD (600 nm) was recorded and 100 μl of each dilution was plated (pour plate method; Benson, 1990) and incubated for 72 hours at 30° C. The number of colony-forming units (CFU) was counted using a colony counter (Bellco® Biotechnology, USA) for plates containing 30 to 300 colonies. A linear regression analysis of mean log CFU/ml against percentage transmittance was performed (Appendix 3), to test the significance of the relationship. From the fitted equation, percentage transmittance values corresponding to 10⁷, 10⁸ and 10⁹ CFU/ml were determined (Appendix 3).

Effect of Cultivar, Age and Inoculum Concentration on Resistance to Bacterial Blight-(Bacterial Blight Experiment 1)

Four A. andraeanum (Hort.) cultivars (Table 4.1) with known levels of field resistance to systemic bacterial blight disease were tested at different ages viz. ‘Lydia’ (2, 2.5, 3 years), ‘Rosa’ (1.5, 2, 3 years), ‘Tropical’ (2, 2.5, 3, 5 years) and ‘Success’ (1.5, 2, 3 years) at three concentrations (10⁷, 10⁸, and 10⁹ CFU/ml) of bacteria during the wet season (December 2002). The plants were initially grown using standard methods. The plants were transferred to pots (21 cm diameter and 22 cm deep) containing peat moss (Premier Horticulture Ltee, Riviere-du-Loup, Quebec) at the centre and coconut husk all around. The cultivars and inoculum concentrations were factorially structured with the ages nested within cultivars. The experiment was replicated twice in a completely randomized design. Inoculations were performed according to the method of Fukui et al. (1998, supra) with some modifications. Plants of each cultivar were injected with 100 μl of each concentration in the cut-petiole of the second youngest leaf (one inoculation per plant). Plants were watered three times a day. Time to systemic infection, time to death and proportion of dead plants per cultivar were recorded once a week over four months. The GFP fluorescence was recorded regularly from sections of leaf lamina and petiole (sections macerated in sterile distilled water, placed on a slide and viewed under the ×63 lens of the Leica fluorescence microscope, blue light) to determine time to systemic infection and at the end, on all non-symptomatic plants to determine whether there was symptomless infection of plants. The presence of the bacteria was readily identified as they emitted strong green fluorescence, which was very different from all other types of fluorescence observed. Based on the study and using time of death of the infected plants as the criterion, the optimum concentration that best differentiated the susceptibility levels of anthurium cultivars to bacterial blight disease was determined.

Identification of Resistance to Systemic Infection by X. Campestris Pv. Dieffenbachiae in Anthurium

Bacterial Blight Experiment 2

Eighteen new cultivars (Table 1) plus the four cultivars used in bacterial blight Experiment 1 were evaluated during the dry season for their resistance to bacterial blight disease using the injection method (100 μl of 10⁹ CFU/ml inoculum), as described above. The four cultivars from Experiment 1 were used as controls to verify the repeatability of the technique. The experiment was performed in a randomized complete block design with three replications, the replications performed over time. Each replicate consisted of three plants per cultivar. Results were recorded as described above.

ANOVA was performed to test whether there were significant cultivar and block differences (P≦0.05) for the 22 cultivars evaluated (NCSS statistical package). Tukey's multiple range test was carried out to separate cultivar differences.

Time to death and time to infection were correlated with the proportion of dead plants and proportion of infected plants using Pearson's product moment correlation (NCSS statistical package) to test whether the linear relationship between them remained similar to that obtained from the optimization experiment.

Average time to death over seasons and the proportion of dead plants over seasons for the four anthurium cultivars evaluated during both the wet and dry seasons using the optimized inoculum density (10⁹ CFU/ml) were correlated using Pearson's product moment correlation (NCSS statistical package) to test for repeatability.

Proportion of dead plants and time (weeks) to death for the 22 cultivars evaluated were correlated with observed field resistance values in Trinidad using Spearman's rank correlation (NCSS statistical package).

Experiment 3

Thirty-four other parental cultivars (Table 1) were screened in the wet season using the injection method as described above. The cultivars, ‘Lydia’ and ‘Tropical’ (part of bacterial blight Experiment 1) were used as controls. The experiment was set in a completely randomized design with 5-9 replications per cultivar based on availability.

Optimizing the Screening Method to Evaluate Resistance to Foliar Infection of Anthurium Andraeanum (Hort.) by X. Campestris Pv. Dieffenbachiae

Experiment 4 (Early Wet Season)

Fourteen anthurium cultivars (Table 1) were used in this experiment, of which field values for 13 were known (through the survey). Three fully opened, immature leaves (stage 2) with no scratches were used per cultivar. Leaves were taken to the Bacteriology laboratory of the University in an air-conditioned vehicle. They were cut under water and placed in conical flasks containing distilled water.

Inoculations were based on a leaf-disc vacuum-infiltration method. Five discs of size 5.6 cm² (2.67 cm in diameter) were randomly cut from each leaf using a cork borer. Each set of five discs was vacuum infiltrated (3-15 seconds depending on the cultivar) with a bacterial suspension (X_{4T2}) containing either 10⁸ or 10⁹ CFU/ml based on the percentage-transmittance-time curve developed earlier. The initial infiltration was kept as uniform as possible.

The original infiltration area of each disc was traced onto clear plastic and later onto brown paper. This was used as a covariate to correct final lesion size. The discs were placed width-wise in plastic trays (dimensions: 54 cm×28 cm×6.5 cm; K10, Canada) lined with two layers of moist paper towel and labeled. The inoculum treatments were kept separate (in trays) and arranged in a randomized complete block design with three replications, and five discs per replicate. The trays were placed individually in large transparent polythene bags in a room with temperature set at 30° C. until total spread of lesions for all cultivars was obtained. For each inoculum density the following variables were measured: the lesion size at 6, 9, 12 and 16 days (lesion size traced onto clear plastic and later onto brown paper) and time taken for lesion expansion to cover the entire disc (TLS). Lesion size was measured using a leaf area meter (ΔT Area Meter MK2 model, Delta-T Devices, Burwell Cambridge, England). Data were analyzed using ANOVA with original infiltration area as a covariate. For each measure of resistance the following were calculated: LSD_{(0.05)}, coefficient of variation, range and index of differentiation. Index of differentiation was calculated as the coefficient of variation between cultivars divided by the coefficient of variation within cultivars.

The rate of lesion expansion over time (slope) was measured for each treatment combination and each replicate by linear regression analysis, keeping the intercept as (0,0). The original infiltration area was subtracted from the lesion size prior to regression analysis. The slopes were subjected to ANOVA to determine the effect of cultivar and inoculum density on the rate of lesion expansion. The LSD_{(0.05)} and index of differentiation were calculated as before.

Based on the most discriminating variable, mean values for cultivars for the two inoculation densities were calculated. The mean values for 13 cultivars from each inoculum density were correlated (Spearman's rank correlation, NCSS statistical package) with field resistance values obtained through the survey to determine the validity of the measure at the two inoculum densities.

Based on the optimum inoculum density, a one-way ANOVA was carried out to determine if there were significant differences between the cultivars evaluated (NCSS statistical package).

Experiment 5 (Late Wet Season)

The experiment was repeated using the same set of fourteen cultivars in the late wet season (October 2003) using the leaf-disc vacuum-infiltration technique only with the optimized inoculum concentration (10⁸ CFU/ml). The experiment was placed in a randomized complete block design with three replicates, and five leaf discs per replicate. Results were recorded as described before in the early wet season.

Data were analyzed as for bacterial blight Experiment 1. Repeatability of the results in this trial compared to the previous trial was assessed by linear regression analysis of time taken for lesion expansion to cover the leaf disc (TLS) (trial 1 vs. trial 2) and by comparison of regression lines (TLS trial 1 vs. Mean TLS; TLS trial 2 vs. Mean TLS).

Experiment 6−Repeatability of Screening Method Over Seasons:

Wet Season Trial

Fourteen anthurium cultivars of which five (‘Local Orange’, ‘Lydia’, ‘Mirjam’, ‘Pierrot’ and ‘Venus’) were already evaluated for foliar resistance to BB earlier in the wet season were evaluated using the optimized screening method and the measure, TLS. The experiment was set in a randomized complete block design with three replicates and five leaf discs per replicate.

Covariance ANOVA (initial infiltration area was used as the covariate) was performed to determine whether cultivar differences were significant at P≦0.05 (NCSS statistical package).

Dry Season Trial

The same set of 14 cultivars (Table 1) was evaluated for resistance to foliar bacterial blight disease in the early dry season (February 2004) using the leaf-disc vacuum-infiltration technique and optimized inoculum concentration (10⁸ CFU/ml). The experiment was set in a randomized complete block design with three replicates and three leaf discs per replicate. Time to total lesion spread (TLS) was measured for each cultivar as described above.

Covariance analysis (initial infiltration area was used as the covariate) was performed to test whether there were significant differences between cultivars (NCSS statistical package). Covariance analysis (GLIM ANOVA) was carried out to determine whether there were significant cultivar, season and cultivar-season interactions for the 14 cultivars (NCSS statistical package).

Correlation analysis was performed between mean wet season and dry season times to total lesion spread (Pearson's product moment correlation; Spearman's rank correlation) to determine whether there was a strong association between seasons (NCSS statistical package).

Comparison of linear regression lines (COLR program) was performed to determine whether the regression lines obtained for mean TLS in each season could be explained by a single regression line.

Evaluation of 27 Anthurium Cultivars for Resistance to Foliar Bacterial Blight Disease in the Late Wet Season

Twenty-seven anthurium cultivars (Table 1) were evaluated for resistance to foliar bacterial blight disease during the wet season (October 2003) using the optimized leaf-disc vacuum-infiltration procedure. The experiment was set in a randomized complete block design with three replications and five leaf discs per replicate. TLS was measured for each cultivar.

All anthurium cultivars evaluated for resistance to foliar infection by bacterial blight disease were categorized based on mean TLS as follows: susceptible (0-8 days), tolerant (>8-12 days), and resistant (>12 days).

Mechanism of Foliar Resistance to Bacterial Blight Disease

Fukui et al. (Fukui et al, 1996, Applied and Environ. Microbiol., 1021-1028) suggested that resistance to bacterial blight disease was due to prevention of bacterial multiplication. Leaf discs of three parental cultivars viz: ‘Local Orange’, ‘Champagne’ and ‘Pierrot’ (susceptible, moderately susceptible, and resistant respectively from the wet season trials) were vacuum infiltrated as described before with the 10⁸ CFU/ml inoculum strength in the dry season trial. Three leaf discs were used per replicate and three replicates were used for each cultivar. On days 5, 7, and 10 respectively of the experiment, one replicate for each cultivar was surface-sterilized with 70% ethanol and macerated in 3 ml of sterile distilled water. Serial dilutions were performed according to Benson (Benson, 1990, Microbial Applications, a Laboratory manual in General Microbiology, 5d ed. USA: Wm. C. Brown Publishers). One hundred microliters of each dilution were pour-plated with Luria agar. After 48 hours, bacterial colonies were counted with a bacterial colony counter.

Log₁₀CFU/ml was calculated for each measurement of CFU/ml for each cultivar. A one-way ANOVA (cultivar and time treatments) was conducted to see whether there were significant differences for bacterial numbers between cultivars over time with each time measurement as one replicate (NCSS statistical package).

Relationship Between Systemic and Foliar Resistance in Anthurium Cultivars

Fukui et al. (1998, supra) indicated that resistance to bacterial blight disease is at two levels, a systemic and a foliar. The mean time to total lesion spread for each of the fourteen anthurium cultivars evaluated for resistance to foliar bacterial blight disease over the wet and dry seasons was calculated. These values were correlated (Pearson's product moment correlation) with the mean time to death and proportion of dead plants for each of these 14 cultivars evaluated for resistance to bacterial blight disease at the systemic level (NCSS statistical package).

Results

Transformed Isolate of X. Campestris Pv. Dieffenbachiae

The success of transformation with the plasmid ‘p519 ngfp’ was confirmed by plasmid isolation and characterization. The transformed colonies of X. campestris pv. dieffenbachiae were morphologically identical to the non-transformed counterparts but fluoresced in blue light when observed under a Leica fluorescence microscope.

There were no significant differences (P≦0.05) in lesion size elicited by the transformed compared to the untransformed isolate, over a 20-day period (Table 2), and the correlation between them was high (r=0.99). A comparison of regressions of lesion expansion vs. time for the transformed and untransformed isolates (Table 3) showed that they can be described by a single regression line (y=5.053x−18.32). These observations indicate that aggressiveness of the bacterial blight isolate was not altered by transformation.

TABLE 2
Aggressiveness of transformed vs. untransformed
X. campestris pv. dieffenbachiae over time
on the leaves of anthurium cultivar, ‘Fantasia’

| | Lesion size (cm²)^{a} | | | | | | |
|---|---|---|---|---|---|---|---|
| | Day | Day | | | | | |
| Isolate | 0 | 9 | Day 11 | Day 13 | Day 16 | Day 18 | Day 20 |
| N-XCD^{b} | 8.58 | 8.58 | 10.30 | 35.82 | 49.80 | 68.68 | 90.62 |
| T-XCD^{c} | 9.04 | 9.60 | 11.08 | 32.76 | 62.78 | 88.40 | 136.68 |
| Mean | 8.81 | 9.09 | 10.69 | 34.29 | 56.29 | 78.54 | 113.65 |
| SED | 0.814 | 1.130 | 2.549 | 15.507 | 35.934 | 51.742 | 63.988 |
| Sig- | NS^{d} | NS | NS | NS | NS | NS | NS |
| nificance | | | | | | | |

^{a}Lesion size for each isolate per day was based on 5 replications.
^{b,c}Untransformed and transformed Xanthomonas campestris pv. dieffenbachiae, respectively
^{d}differences between bacterial isolates were not significant at P ≦ 0.05.

TABLE 3
Comparison of regression of lesion size vs. time (aggressiveness) for
transformed and untransformed X. campestris pv. dieffenbachiae
isolates
Analysis of variance

| Source | DF | SS | MS | VR |
|---|---|---|---|---|
| Residual (pooled) | 10 | 6398.3 | 639.8 | |
| Between slopes | 1 | 450.4 | 450.4 | 0.704 |
| Residual (//lines) | 11 | 6848.6 | 622.6 | |
| Between adj. means | 1 | 434.1 | 434.1 | 0.697 |
| Residual (overall) | 12 | 7282.8 | 606.8 | |
| Overall regression | 1 | 13771.2 | 13771.2 | |
| Total | 13 | 21053.9 | | |

Screening Method: Systemic Resistance to Bacterial Blight Disease (BB)

Effect of Plant Age and Inoculum Concentration

The cultivars ‘Tropical’ and ‘Success’ showed high levels of resistance to BB (Table 4) and did not become infected at the end of the experiment (16 WAI), except for a single plant of ‘Success’ that became infected at the inoculum density of 10⁹ CFU/ml. There was no evidence of symptomless infection as indicated by the lack of green fluorescence in leaves of the non-infected plants.

On the contrary, the cultivars ‘Lydia’ and ‘Rosa’ showed early symptoms 1-2 WAI and can be classified as susceptible. More often than not, there was a quick decline and death immediately following the first evidence of symptoms. Infected plants consistently showed green fluorescence in all leaves. Occasionally, at lower concentrations, particularly in the cultivar, ‘Rosa’, a few plants escaped infection or plant death was delayed. There was no evidence that age of plants (1.5-5.0 years) had an effect on the time to first symptom or time to death (crown infection). Changes in concentration of inoculum did not affect the time to first symptom or death in ‘Lydia’, but high concentrations reduced the chances of escape in ‘Rosa’.

TABLE 4
Effect of cultivar, plant age and inoculum density (CFU/ml) on time to
symptom, time to death and proportion of dead plants on four
cultivars of Anthurium andraeanum (Hort.).

| | | Time to | | | Time to | | | | | | | | |
|---|---|---|---|---|---|---|---|---|---|---|---|---|---|
| Cul- | | symptoms | | | death | | | Proportion of plants | | | | | |
| ti- | Age | (weeks) | | | (weeks) | | | symptomatic dead | | | | | |
| var^{a} | (yrs) | 10⁷ | 10⁸ | 10⁹ | 10⁷ | 10⁸ | 10⁹ | 10⁷ | 10⁸ | 10⁹ | 10⁷ | 10⁸ | 10⁹ |
| Rosa | 1.5 | 10 | 10 | 7 | 10 | 10 | 7 | | | | | | |
| | 1.5 | 13 | 9 | 9 | 13 | 9 | 9 | 1.0 | 1.0 | 1.0 | 1.0 | 1.0 | 1.0 |
| | 2.0 | 15 | ^{b}— | 14 | 15 | — | 14 | | | | | | |
| | 2.0 | 14 | 12 | 11 | — | 12 | 11 | 1.0 | 0.5 | 1.0 | 0.5 | 0.5 | 1.0 |
| | 3.0 | — | 14 | 11 | — | 14 | 11 | | | | | | |
| | 3.0 | 14 | 9 | 14 | 13 | 9 | 14 | 0.5 | 1.0 | 1.0 | 0.5 | 1.0 | 1.0 |
| Trop- | 2.0 | — | — | — | — | — | — | | | | | | |
| ical | 2.0 | — | — | — | — | — | — | 0.0 | 0.0 | 0.0 | 0.0 | 0.0 | 0.0 |
| | 2.5 | — | — | — | — | — | — | | | | | | |
| | 2.5 | — | — | — | — | — | — | 0.0 | 0.0 | 0.0 | 0.0 | 0.0 | 0.0 |
| | 3.0 | — | — | — | — | — | — | | | | | | |
| | 3.0 | — | — | — | — | — | — | 0.0 | 0.0 | 0.0 | 0.0 | 0.0 | 0.0 |
| | 5.0 | — | — | — | — | — | — | | | | | | |
| | 5.0 | — | — | — | — | — | — | 0.0 | 0.0 | 0.0 | 0.0 | 0.0 | 0.0 |
| Suc- | 1.5 | — | — | — | — | — | — | | | | | | |
| cess | 1.5 | — | — | — | — | — | — | 0.0 | 0.0 | 0.0 | 0.0 | 0.0 | 0.0 |
| | 2.0 | — | — | 10 | — | — | 10 | | | | | | |
| | 2.0 | — | — | — | — | — | — | 0.0 | 0.0 | 0.5 | 0.0 | 0.0 | 0.5 |
| | 3.0 | — | — | — | — | — | — | | | | | | |
| | 3.0 | — | — | — | — | — | — | 0.0 | 0.0 | 0.0 | 0.0 | 0.0 | 0.0 |
| Lydia | 2.0 | 10 | 11 | 14 | 10 | 11 | 14 | | | | | | |
| | 2.0 | 10 | 14 | 9 | 10 | — | 9 | 1.0 | 1.0 | 1.0 | 1.0 | 0.5 | 1.0 |
| | 2.5 | 11 | 7 | 9 | 11 | 9 | 10 | | | | | | |
| | 2.5 | 12 | 8 | 8 | 12 | 11 | 10 | 1.0 | 1.0 | 1.0 | 1.0 | 1.0 | 1.0 |
| | 3.0 | 14 | 9 | 7 | 15 | 9 | 11 | | | | | | |
| | 3.0 | 11 | 12 | 11 | 23 | 12 | 11 | 1.0 | 1.0 | 1.0 | 1.0 | 1.0 | 1.0 |

^{a}Only cultivar was significant at P ≦ 0.01. Standard error:time to death = 2.99.
^{b}‘_’ Means plant did not become infected nor died.

These data further demonstrate the suitability of the fluorescent BB method for identifying disease resistant plants.

The results indicate that cultivar effects were the most important for resistance. Since age of cultivar did not seem to influence resistance to BB, segregating populations of anthurium can be screened as early as 1.5 years for resistance to BB. A higher concentration of 10⁹ CFU/ml may be better suited to avoid escape, especially when screening unique progeny genotypes of segregating generations obtained through inter-specific hybridization of anthurium species.

Ability to Differentiate Between Cultivars for Resistance to BB in the Dry Season

Bacterial Blight Experiment 1

The differences between cultivars for time to first symptom, time to death, proportion of symptomatic plants and proportion of dead plants were significant (P<0.001) (Table 5). There was a perfect correspondence (r=1) between the proportion of symptomatic plants and the proportion of plants that died in all cultivars except ‘Venus’ and ‘Spirit’, where a small number of symptomatic plants did not die at the end of the experiment. Similarly, there were high correlations between time to first symptom development and time to death (r=1.00); time to first symptom development and proportion of symptomatic plants/dead plants (r=−0.80); and time to death and proportion of symptomatic plants/dead plants (r=−0.80). The index of differentiation for Tukey's multiple range test was used to separate the cultivars into groups, based on their level of resistance. Those that had 100% death with a quick decline, within 6-10 weeks after inoculation (WAI), were designated as susceptible. Cultivars that did not die and were free of symptoms at the end of the experiment and those that had only a small percentage (<22%) of plants dying but took a very long time to decline (>23 WAI) were designated as resistant. The others, which had a higher percentage of plants declining (>44%) with a relatively quick decline, between 10-12 WAI, were regarded as tolerant. The tolerant cultivars include ‘Honduras’, ‘Sonate’, ‘Spirit’ and ‘Success’.

The results show that time taken to death and the proportion of plants that die can be used to discriminate among resistance levels. Each of the four variables measured was similar, suggesting that any of the four may be used to distinguish cultivar differences in the levels of resistance to systemic infection by BB.

TABLE 5
Time to symptom development/death, proportion of
symptomatic/dead plants for 22 anthurium cultivars inoculated with
X. campestris pv. dieffenbachiae (10⁹ CFU/ml) under shade-house
conditions during the dry season.

| | Time to | | | | | |
|---|---|---|---|---|---|---|
| | symptom^{b} | | Prop. | Time to death | | Prop. |
| | (weeks) | | Infected | (weeks) | | Dead |
| Cultivar^{a} | Range | Mean | plants^{c} | Range | Mean | plants^{c} |
| Acropolis | — | — | 0.00 | — | — | 0.00 |
| Champagne | 7-11 | 9.0 | 1.00 | 7-11 | 9.0 | 1.00 |
| Cheers | 6-12 | 9.2 | 1.00 | 6-12 | 9.2 | 1.00 |
| Cuba | 5-8 | 6.1 | 1.00 | 5-8 | 6.1 | 1.00 |
| Fantasia | 6-8 | 7.1 | 1.00 | 6-8 | 7.1 | 1.00 |
| Gloria | 4-11 | 6.9 | 1.00 | 4-11 | 6.9 | 1.00 |
| Hawaii | 6-10 | 7.7 | 1.00 | 6-10 | 7.7 | 1.00 |
| Honduras | 9-12 | 10.5 | 0.44 | 9-12 | 10.5 | 0.44 |
| Ibara | — | — | 0.00 | — | — | 0.00 |
| Kalapana | 20-27 | 22.7 | 0.22 | 20-27 | 22.7 | 0.22 |
| Local Orange | — | — | 0.00 | — | — | 0.00 |
| Lydia | 6-11 | 7.7 | 1.00 | 6-11 | 7.7 | 1.00 |
| Midori | 8-11 | 9.3 | 1.00 | 8-11 | 9.3 | 1.00 |
| Mirjam | — | — | 0.00 | — | — | 0.00 |
| Pierrot | 5-14 | 8.8 | 1.00 | 5-14 | 8.8 | 1.00 |
| Rosa | 7-13 | 8.6 | 1.00 | 7-13 | 8.6 | 1.00 |
| Senator | 27 | 27.0 | 0.11 | 27 | 27.0 | 0.11 |
| Sonate | 8-13 | 9.8 | 0.56 | 8-13 | 9.8 | 0.56 |
| Spirit | 9-15 | 12.5 | 0.44 | 9-14 | 11.7 | 0.33 |
| Success | 7-15 | 9.8 | 0.56 | 7-15 | 9.8 | 0.56 |
| Tropical | — | — | 0.00 | — | — | 0.00 |
| Venus | 26-27 | 26.5 | 0.22 | 26 | 26 | 0.11 |
| Sig. (P ≦) | | 0.001 | 0.001 | | 0.001 | 0.001 |
| Mean | | 11.8 | 0.84 | | 11.7 | 0.84 |
| SEM | | 0.88 | 0.090 | | 0.88 | 0.087 |
| ID^{d} | | 4.42 | 4.47 | | 4.39 | 4.68 |

^{a}Cultivar differences were significant at P ≦ 0.01, but block differences were not significant.
^{b}Mean and range for time to symptom and death were based on only the plants that died.
^{c}Arcsin proportion of infected & dead plants were used in analysis.
^{d}Index of differentiation.

Repeatability of the Screening Method

There was a strong and significant (P≦0.05) correlation (Table 6) between the mean time taken to death in the late wet and dry seasons (r=−0.81) and the proportion of dead plants in the late wet and dry seasons (r=0.93). Therefore, both measures of resistance were repeatable over seasons. The season effect (blocks) was not significant in the ANOVA test, indicating that the effect of season did not significantly affect the results of screening.

TABLE 6
Average time to death and proportion of dead plants for four
A. andraeanum (Hort.) cultivars evaluated over both
the wet and dry seasons.

| | | Average time to death | Prop. dead |
|---|---|---|---|
| Cultivar | Trial | (weeks) | plants |
| Rosa | Late wet Season | 11.0 | 1.00 |
| | Dry Season | 8.6 | 1.00 |
| Tropical | Late wet Season | — | 0.00 |
| | Dry Season | — | 0.00 |
| Success | Late wet Season | 10.0 | 0.17 |
| | Dry Season | 9.8 | 0.56 |
| Lydia | Late wet Season | 10.8 | 1.00 |
| | Dry Season | 7.7 | 1.00 |

Validity of the Screening Method

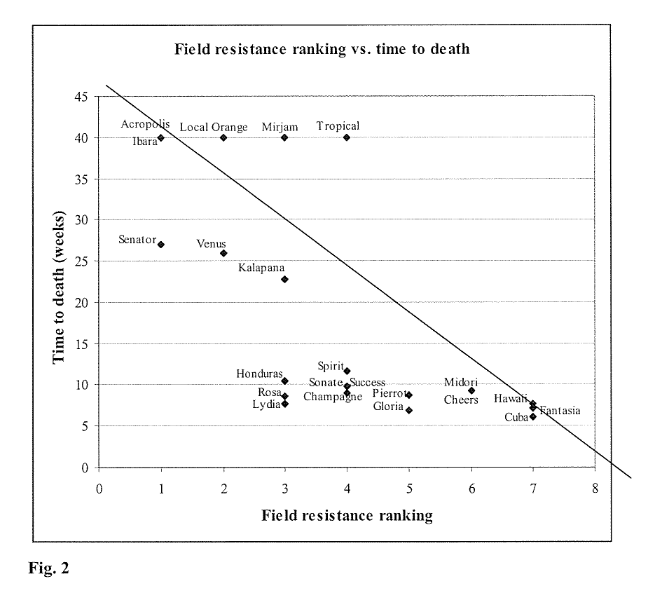

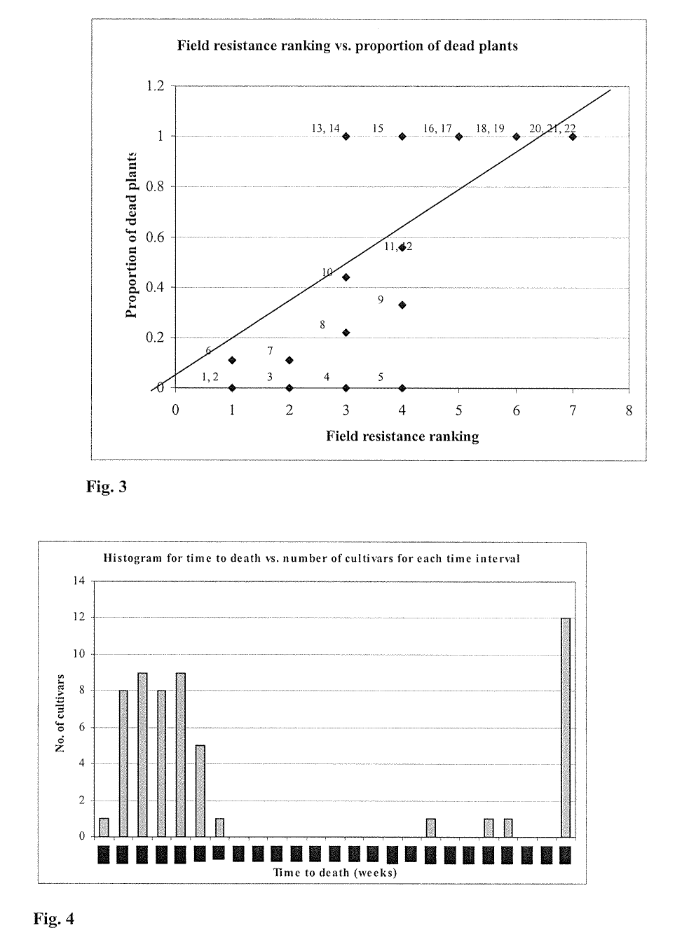

Spearman's rank correlation between cultivar resistance values obtained from the dry season experiment and field resistance reported for the 22 cultivars by farms in Trinidad (Avey, pers. comm., 2001; Table 1) were strong and significant (P<0.05) for time to death (r=−0.70; FIG. 2) and proportion of dead plants (r=0.75; FIG. 3). FIG. 2 and FIG. 3, show that there was general agreement in the field rankings and experimental ranking based on time to plant death and proportion of dead plants. Some inconsistencies seen with ‘Honduras’, ‘Rosa’, ‘Lydia’, ‘Mirjam’ and ‘Tropical’ may be a result of the inability of farmers to distinguish between systemic and foliar resistance.

Identification Of Systemic Resistance To Bacterial Blight Disease Table 7 shows the proportion of plants that died (PPD) and mean time to plant death (TPD) for 34 other cultivars of anthurium using the screening method developed and described above. Seven cultivars, ‘Bianco,’ ‘Cotopaxi,’ ‘Farao,’ ‘Local Cup White,’ ‘Local Pink,’ ‘Local Mina White,’ and ‘Sweety’ with no deaths, were categorized as resistant. The susceptible cultivars (21), as before, had 100% plant death, and declined rapidly between 6.7-10.4 WAI. In FIG. 2, cultivars that did not die at all were given a time of 40 weeks to death to separate them clearly from the others.

TABLE 7
Mean time to death (weeks) for 34 cultivars of anthurium under
artificial inoculation with Xanthomonas campestris pv. dieffenbachiae
during the wet season.

| | Time to | Proportion |
|---|---|---|
| | death^{a} | of dead |
| Cultivar | (weeks) | plants |
| Alexis | 7.8 | 1.00 |
| Andes 115 | 6.4 | 1.00 |
| Avonette | 10.4 | 1.00 |
| Bianco | — | 0.00 |
| Cotopaxi | — | 0.00 |
| Cross 163 | 7.5 | 1.00 |
| Cross 2135 | 6.6 | 1.00 |
| Cross 3962 | 10.3 | 1.00 |
| Cross 4038 | 7.7 | 1.00 |
| Cross 793 | 5.8 | 1.00 |
| Cumbia | 9.7 | 1.00 |
| Farao | — | 0.00 |
| Fla Range | 7.2 | 1.00 |
| Furore | 8.5 | 1.00 |
| Laguna | 8.0 | 1.00 |
| Lima | 8.4 | 1.00 |
| Linda | 7.0 | 1.00 |
| Local Cup White | — | 0.00 |
| Local Mina Pink | 11.0 | 0.80 |
| Local Mina Red | 10.0 | 1.00 |
| Local Mina | — | 0.00 |
| White | | |
| Local Pink | — | 0.00 |
| Local Whitish | 10.0 | 0.22 |
| Pink | | |
| Lunette | 7.0 | 1.00 |
| Margaretha | 7.9 | 1.00 |
| Minuet | 9.3 | 1.00 |
| Patricia | 6.7 | 1.00 |
| President | 8.5 | 0.50 |
| Rapido | 10.5 | 0.67 |
| Safari | 9.0 | 0.33 |
| Sweety | — | 0.00 |
| Tequila | 7.0 | 1.00 |
| Terra | 9.3 | 0.44 |
| Victoria | 8.8 | 1.00 |
| Sig. (P ≦ 0.05) | 0.001 | 0.001 |
| Mean | 8.1 | 0.68 |
| SEM | 0.65 | 0.162 |
| ID^{b} | 1.26 | 1.62 |

‘—’ Means that plants for this cultivar did not die.
^{a}Mean time to death is based only on the plants that died.
^{b}Index of differentiation.

Six cultivars had less than 100% plant death, but varied in the proportion of dead plants (22%-80%). The plants that died, however, declined rapidly between 8.5 to 11 WAI, which were not significantly (P≦0.05) different from the susceptible category based on the Tukey-Kraemer test. Hence, unlike the previous experiment, the correlation (r=−0.40; P≦0.05) between PPD and TPD was weak, although it was significant (P<0.05). This category therefore, is designated as tolerant. Whereas the resistant and susceptible categories can be easily discerned using either the PPD or TPD, the tolerant category is largely distinguished by the proportion of plants that died. Hence, the index of differentiation was higher for PPD than for TPD (Table 7).

Table 8 places the 56 anthurium cultivars screened into three categories based on their systemic resistance to BB. Approximately 55% (31 cultivars) were categorized as highly susceptible. This group of cultivars exhibited 100% plant death, with a quick decline, usually between 6-10.4 WAI. Around 18% fell into the tolerant category, with cultivars belonging to this category showing less than 100% plant death and taking a longer time to succumb to the disease, 10.5 to 20 weeks. The plants in the resistant category (27%) either did not succumb to the disease (21%) or succumbed less frequently than the tolerant category (6%), with those dying taking 20 to 30 weeks to die.

The frequency distribution for time to death (FIG. 4) was skewed to the right and was discontinuous. In FIG. 4, cultivars with no plants that died were given a dummy value of 30 to distinguish them from the other categories. The tolerant cultivars clustered with the susceptible cultivars into one continuous group while the resistant category clustered separately. The frequency distribution for proportion of dead plants (0, 1-20, 21-40, 41-60, 61-80, 81-99, 100%) (FIG. 5 [4.5]) also showed a discontinuous distribution with the resistant and tolerant cultivars clustering together (0-99%). The cultivar, ‘Kalapana’, although resistant to systemic infection by BB, clustered with the tolerant ones. Susceptible cultivars clustered separately, each having 100% dead plants. Pearson's product moment correlation (NCSS statistical package) between time to death and proportion of dead plants for 44 of the 56 cultivars (cultivars that did not have any dead plants were excluded) was high and significant (r=−0.73; P≦0.05), further suggesting that both measures of resistance are governed by the same mechanism.

TABLE 8
Level of systemic resistance (susceptible, tolerant and resistant) of
56 anthurium cultivars to bacterial blight disease based on the
average time to death in weeks
Cultivars

| Susceptible | Tolerant | Resistant |
|---|---|---|
| (0-10 weeks) | (11-20 weeks) | (21-30 weeks) |
| Alexis | Honduras | Acropolis* |
| Avonette | Local Mina Pink | Bianco* |
| Andes 115 | Local Whitish Pink | Cotopaxi* |
| Champagne | President | Farao* |
| Cheers | Rapido | Ibara* |
| Cross 163 | Safari | Kalapana |
| Cross 2135 | Sonate | Local Cup White* |
| Cross 3962 | Spirit | Local Mina White* |
| Cross 4038 | Success | Local Orange* |
| Cross 793 | Terra | Local Pink* |
| Cuba | | Mirjam* |
| Cumbia | | Senator |
| Fantasia | | Sweety* |
| Fla Range | | Tropical* |
| Furore | | Venus |
| Gloria | | |
| Hawaii | | |
| Laguna | | |
| Lima | | |
| Linda | | |
| Local Mina | | |
| Red | | |
| Lunette | | |
| Lydia | | |
| Margaretha | | |
| Midori | | |
| Minuet | | |
| Patricia | | |
| Pierrot | | |
| Rosa | | |
| Tequila | | |
| Victoria | | |

*Plants for that cultivar neither died nor became infected.

Screening Method: Foliar Resistance to Bacterial Blight Disease

Optimum Inoculum Concentration and Measure of Resistance

Time taken for the expanding lesion to cover the entire leaf disc, denoted as time to total lesion spread (TLS), had the largest index of differentiation among the various measures of resistance calculated (Table 9). Furthermore, the index of differentiation for TLS at the inoculum density of 10⁸ CFU/ml was approximately twice that at 10⁹ CFU/ml.

The leaf-disc inoculation method using inoculum at a density of 10⁸ CFU/ml was best able to differentiate levels of foliar resistance among anthurium cultivars. The second most differentiating attribute was ‘lesion size at day 6’. The correlation coefficient for TLS and lesion size at day 6 was large (r=−0.75) and significant (P<0.01).

TABLE 9
Differentiation index of two measures of foliar resistance to bacterial
blight disease of anthurium (a) time taken for the expanding lesion to
cover the entire leaf (TLS); (b) lesion size at 6, 9, 12 and 16 days; and
(c) mean lesion size per day (slope) in response to inoculation at two
inoculum densities (10⁸ and 10⁹ CFU/ml) during the early wet season (trial 1).

| | 10⁸ CFU/ml | | | | | | 10⁹ CFU/ml | | | | | |
|---|---|---|---|---|---|---|---|---|---|---|---|---|
| | | Lesion size at day | | | | Mean | | Lesion size at day | | | | Mean |
| | TLS^{a} | (cm²) | | | | slope | TLS | (cm²) | | | | slope |
| Statistic | (days) | 6 | 9 | 12 | 16 | (cm²/day) | (days) | 6 | 9 | 12 | 16 | (cm²/day) |
| Signifi- | 0.001 | 0.001 | 0.001 | 0.001 | ^{b}NS | 0.001 | 0.001 | 0.001 | 0.001 | 0.001 | 0.001 | 0.001 |
| cance | | | | | | | | | | | | |
| (P ≦) | | | | | | | | | | | | |
| Mean | 10.42 | 3.36 | 4.14 | 4.63 | 4.98 | 0.28 | 10.89 | 4.1 | 4.21 | 4.77 | 5.18 | 0.24 |
| SEM | 0.635 | 0.297 | 0.41 | 0.383 | 0.211 | 0.041 | 0.942 | 0.243 | 0.376 | 0.504 | 0.49 | 0.053 |
| CV (%) | 23 | 30 | 25 | 16 | 6 | 24 | 33 | 80 | 55 | 48 | 28 | 38 |
| Range | 5.6-15.7 | 2.56-5.98 | 3.49-5.62 | 3.98-6.06 | 5.06-5.79 | 0.18-0.64 | 6.0-14 | 0.24-2.67 | 0.67-3.25 | 1.17-4.00 | 0.86-3.88 | 0.09-0.46 |
| ^{c}ID | 6.74 | 4.89 | 2.2 | 2.29 | 1.46 | 4.6 | 3.64 | 4.26 | 2.9 | 2.38 | 2.6 | 2.85 |

^{a}Time taken for the expanding lesion to cover the entire leaf disc.
^{b}not significant.
^{c}index of differentiation was calculated as range/LSD_{(0.05)}.

The correlation between the levels of foliar field resistance to bacterial blight disease, obtained from a farmer survey in Trinidad (Table 1; and TLS, (Table 10) at an inoculum density of 10⁸ CFU/ml, was strong (r=−0.84) and significant (P<0.001), and was much larger than that obtained between field resistance and TLS at inoculum density of 10⁹ CFU/ml (r=−0.30) or that obtained between field resistance and ‘lesion size at day 6’ (r=0.69). As TLS at 10⁸ CFU/ml inoculum density best differentiated amongst the cultivars tested and had the strongest correlation to field resistance, it was the measure of choice to assess resistance to bacterial blight disease in subsequent experiments.

Repeatability within the Wet Season

When the leaf disc inoculation experiment was repeated in the late wet season (Table 11) using an inoculum density of 10⁸ CFU/ml (Xanthomonas campestris pv. dieffenbachiae), again TLS gave the best index of differentiation (Table 12). The standard error of mean TLS was slightly higher for Trial 2 and consequently the indices of differentiation were slightly smaller than for Trial 1.

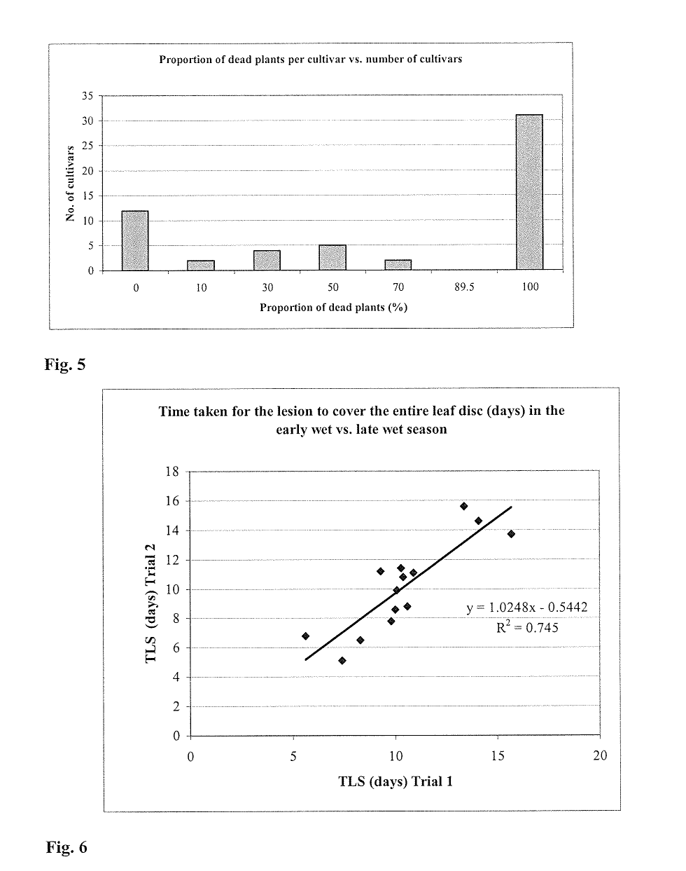

An ANOVA showed that there were no significant differences between cultivar responses in Trial 1 and Trial 2 (Table 4.11). Furthermore, there was a strong correspondence (FIG. 6) between TLS (10⁸ CFU/ml) obtained in the two trials (Pearson's correlation coefficient, r=0.87; Spearman's rank correlation coefficient, r=0.83). A comparison of regression lines ‘mean TLS (Trials 1 and 2) vs. TLS (Trial 1)’ and ‘mean TLS vs. TLS (Trial 2)’ showed that they can be described by a single regression line (Table 4.13). These results showed that the TLS values were repeatable over separate trials.

From Table 11, the cultivars ‘Pierrot’, ‘Rosa’ and ‘Midori’ had the longest mean TLS whereas ‘Local Orange’, ‘Fantasia’ and ‘Gloria’ had the shortest times during the wet season.

TABLE 10
Time taken for the expanding lesion to cover the entire leaf disc (days)
for 14 anthurium cultivars inoculated with 10⁸ and 10⁹ CFU/ml
of X. campestris pv. dieffenbachiae in the early wet season (Trial 1).

Time taken for the lesion to cover the
entire disc (days)

| Cultivar | 10⁸ CFU/ml | 10⁹ CFU/ml |
|---|---|---|
| Cheers | 10.4 | 11.6 |
| Cuba | 10.3 | 13.1 |
| Fantasia | 7.4 | 7.3 |
| Gloria | 8.3 | 11.2 |
| Hawaii | 10.6 | 11.8 |
| Local Orange | 5.6 | 6.0 |
| Lydia | 9.3 | 12.9 |
| Midori | 13.4 | 10.4 |
| Mirjam | 10.1 | 11.9 |
| Pierrot | 14.1 | 14.0 |
| Rosa | 15.7 | 11.4 |
| Senator | 9.8 | 10.7 |
| Tropical | 10.0 | 9.5 |
| Venus | 10.9 | 10.7 |
| SEM | 0.64 | 0.94 |

TABLE 11
Time taken for lesion to cover the entire leaf disc (TLS, in days) for 14
anthurium cultivars inoculated with 10⁸ CFU/ml of X. campestris pv.
dieffenbachiae during the early wet season (Trial 1), late wet season
(Trial 2) and mean TLS for the two experiments.

| | TLS | TLS | Mean TLS of |
|---|---|---|---|
| Cultivar | Trial 1 | Trial 2 | Trials 1 & 2 |
| Cheers | 10.4 | 10.8 | 10.6 |
| Cuba | 10.3 | 11.4 | 10.9 |
| Fantasia | 7.4 | 5.1 | 6.3 |
| Gloria | 8.3 | 6.5 | 7.4 |
| Hawaii | 10.6 | 8.8 | 9.7 |
| Local Orange | 5.6 | 6.4 | 6.0 |
| Lydia | 9.3 | 11.2 | 10.3 |
| Midori | 13.4 | 15.6 | 14.5 |
| Mirjam | 10.1 | 9.9 | 10.0 |
| Pierrot | 14.1 | 14.6 | 14.4 |
| Rosa | 15.7 | 13.7 | 14.7 |
| Senator | 9.8 | 7.8 | 8.8 |
| Tropical | 10 | 8.6 | 9.3 |
| Venus | 10.9 | 11.1 | 11.0 |
| SEM | 0.64 | 0.72 | |

TABLE 12
Differentiation index of two measures of foliar resistance to bacterial
blight (a) time taken for the expanding lesion to cover the entire leaf
(TLS); (b) lesion size at 6, 9, and 12 days; and mean lesion size per
day (slope) in response to inoculation at 10⁸ CFU/ml during the late
wet season (Trial 2).

| | 10⁸ CFU/ml | | | | |
|---|---|---|---|---|---|
| | TLS^{a} | Lesion size at day (cm²) | | | Mean slope |
| Statistic | (days) | 6 | 9 | 12 | (cm²/day) |
| Significance | 0.001 | 0.001 | 0.001 | 0.001 | 0.001 |
| Mean | 10.14 | 3.84 | 4.37 | 5.03 | 0.32 |
| SEM | 0.715 | 0.267 | 0.307 | 0.394 | 0.063 |
| CV (%) | 27 | 22 | 21 | 19 | 30 |
| Range | 5.1-15.6 | 2.55-5.18 | 2.93-5.62 | 3.77-5.72 | 0.14-0.71 |
| ID^{b} | 6.21 | 4.53 | 3.71 | 2.10 | 3.84 |

^{a}time taken for the lesions to entirely cover the leaf disc.
^{b}index of differentiation was calculated as range of cultivars/LSD_{(0.05)}.

TABLE 13
Comparison of linear regression lines, mean TLS vs. TLS in Trial 1
and mean TLS vs. Trial 2, for 14 anthurium cultivars evaluated for
foliar resistance to bacterial blight disease during the wet season.
Analysis of variance

| Source | DF | SS | MS | VR |
|---|---|---|---|---|
| Residual (pooled) | 24 | 14.4067 | 0.6003 | |
| Between slopes | 1 | 1.6819 | 1.6819 | 2.802 |
| Residual (//lines) | 25 | 16.0886 | 0.6435 | |
| Between adj. means | 1 | 0.5714 | 0.5714 | 0.888 |
| Residual (overall) | 26 | 16.6600 | 0.6408 | |
| Overall regression | 1 | 199.2071 | 199.2071 | |
| Total | 27 | 215.8671 | | |

TLS = time taken for the lesions to entirely cover the leaf disc.

Repeatability in the Dry Season

TLS values for 14 cultivars evaluated in the wet and dry seasons are shown in Table 14. TLS was generally higher in the dry season compared to the wet. ‘Cross 3962’, ‘Ibara’ and ‘Local Orange’ showed a more than twofold increase in TLS in the dry than in the wet season. Cultivars ‘Cross 356’, ‘Honduras’, ‘Pierrot’ and ‘Success’ had the highest mean TLS over the two seasons (Table 14). These observations explain the greater problem of this disease in the wet season than in the dry.

An ANOVA of TLS values for the 14 cultivars using seasons as blocks showed that cultivar differences interaction effect was significant only at P<0.05, but the F-value was small. A comparison of regression lines ‘mean TLS (wet & dry) vs. TLS (wet)’ and ‘mean TLS vs. TLS (dry)’ showed that they can be described by two parallel lines (y=1.002x−1.73, wet season; y=1.002x+1.61, dry season), suggesting no cultivar-season interaction (Table 15) and season effects were significant at P<0.001. The cultivar×season interaction effect was significant only at P<0.05, but the F-value was small. A comparison of regression lines ‘mean TLS (wet & dry) vs. TLS (wet)’ and ‘mean TLS vs. TLS (dry)’ showed that they can be described by two parallel lines (y=1.002x−1.73, wet season; y=1.002x+1.61, dry season), suggesting no cultivar-season interaction (Table 15).

TABLE 14
TLS^{a} for leaves of 14 parental Anthurium andraeanum (Hort.)
cultivars in the wet and dry seasons.

| | | | Mean TLS |
|---|---|---|---|
| | Mean TLS | TLS (days) | (days) over |
| | (days) wet | early dry | both wet and |
| Cultivar | season | season | dry seasons |
| Acropolis | 18.0 | 16.0 | 17.0 |
| Champagne | 10.0 | 12.0 | 11.0 |
| Cross 356 | 18.8 | 17.6 | 18.2 |
| Cross 3962 | 7.1 | 18.7 | 12.9 |
| Honduras | 17.2 | 23.4 | 20.3 |
| Ibara | 5.1 | 11.3 | 8.2 |
| Local Orange | 6.0 | 14.2 | 10.1 |
| Local Whitish Pink | 11.1 | 12.3 | 11.7 |
| Lydia | 11.1 | 13.3 | 12.2 |
| Mirjam | 10.1 | 12.1 | 11.1 |
| Pierrot | 14.0 | 20.2 | 17.1 |
| Success | 17.0 | 17.3 | 17.2 |
| Venus | 11.5 | 14.1 | 12.8 |
| Victoria | 11.5 | 11.5 | 11.5 |
| SEM | ^{b}0.87 | ^{c}1.25 | |

^{a}time taken for the lesions to entirely cover the leaf disc.
^{b,c}Means and SE for wet and dry seasons were based on 5 and 3 discs per replicate, respectively.

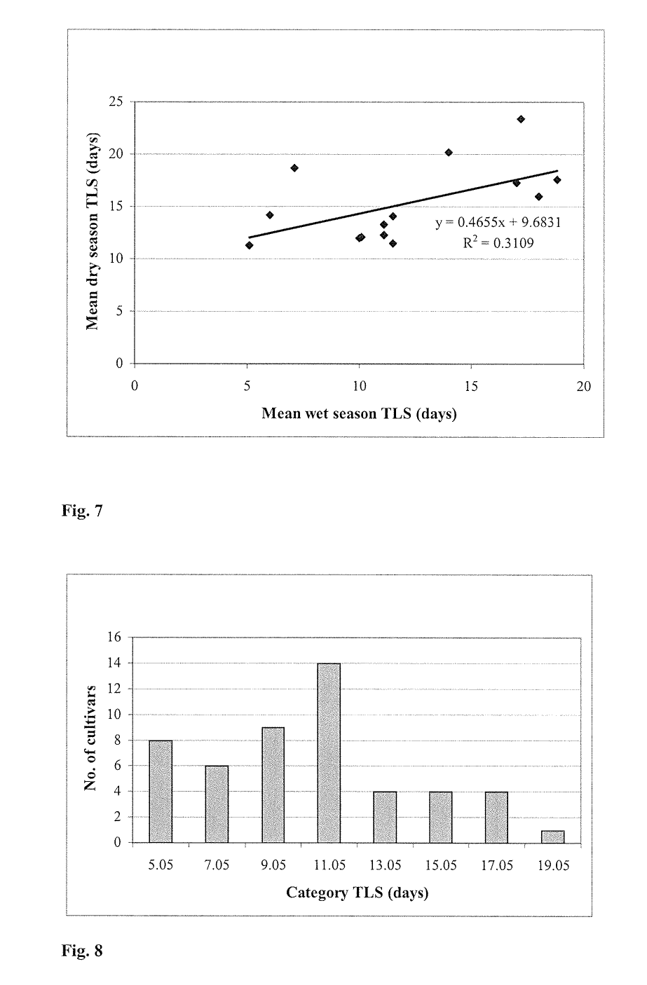

There was a significant correlation (r=0.56, P≦0.05) between TLS for the 14 cultivars evaluated in the wet and dry seasons (FIG. 7), with Pearson's product moment correlation coefficient, r=0.57 and Spearman's rank correlation coefficient, r=0.56. The correlation coefficient for 11 of these cultivars (‘Cross 3962’, ‘Ibara’ and ‘Local Orange’ excluded) was even higher (Pearson's, r=0.75; Spearman, r=0.77). The discrepancy with the three cultivars was perhaps due to the inability to obtain the correct leaf stage due to the restricted number of plants available to choose from.

TABLE 15
Comparison of linear regression lines, mean TLS^{a} vs. TLS in the late
wet season and mean TLS vs. TLS in the dry season, for 14 anthurium
cultivars evaluated for foliar resistance to bacterial blight disease.
Analysis of variance

| Source | DF | SS | MS | VR |
|---|---|---|---|---|
| Residual (pooled) | 24 | 91.8066 | 3.8253 | |
| Between slopes | 1 | 4.7840 | 4.7840 | 1.251 |
| Residual (//lines) | 25 | 96.5906 | 3.8636 | |
| Between adj. means | 1 | 77.8890 | 77.8890 | 20.160 |
| Residual (overall) | 26 | 174.4796 | 6.7108 | |
| Overall regression | 1 | 339.8415 | 339.8415 | |
| Total | 27 | 514.3212 | | |

^{a}time taken for the lesions to entirely cover the leaf disc.

Identification of Foliar Resistance to X. Campestris Pv. Dieffenbachiae in the Wet Season

Twenty-seven other anthurium cultivars were evaluated in the late wet season (Table 16), when the disease is economically most important. The ANOVA indicated differences between cultivars were significant (P<0.05). ‘Kalapana’ was the most susceptible, with a TLS of 4.1 days, whereas ‘Bianco’, (17.1 days) had the longest TLS.

The frequency distribution for TLS of 50 anthurium cultivars evaluated for resistance to foliar BB during the wet season (FIG. 8) was continuous but somewhat skewed towards susceptibility. By day 8, leaf discs of 28% of the cultivars had deteriorated (4-8 days). These may be considered susceptible. Cultivars with TLS of greater than 8 days to 12 days (46%) may be considered tolerant and cultivars with TLS greater than 12 days (26%) may be considered resistant.

TABLE 16
Foliar resistance of 27 Anthurium andraeanum (Hort.) cultivars to
bacterial blight disease during the late wet season.

| | TLS |
|---|---|
| Cultivar | (days) |
| Alexis | 13.8 |
| Andes 115 | 7.5 |
| Bianco | 17.1 |
| Cotopaxi | 10.5 |
| Cross 2135 | 5.3 |
| Cross 2306 | 11.5 |
| Cross 4835 | 11.6 |
| Cumbia | 8.2 |
| Farao | 8.4 |
| Furore | 9.6 |
| Kalapana | 4.1 |
| Laguna | 4.8 |
| Local Mina Pink | 10.8 |
| Local Mina Red | 8.3 |
| Local Mina White | 5.5 |
| Local Pink | 11.6 |
| Minuet | 11.4 |
| Pistache | 8.5 |
| President | 14.2 |
| Rapido | 5.3 |
| Safari | 5.5 |
| Sonate | 12.6 |
| Spirit | 13 |
| Sweety | 7.9 |
| Tequila | 8 |
| Terra | 14.9 |
| Victoria | 11.5 |

TLS = time taken for the lesions to entirely cover the leaf disc.

Mechanism of Foliar Resistance

Differences between cultivars based on log₁₀ CFU/disc were significant at P≦0.05. Table 17 shows the number of colony forming units for three anthurium cultivars evaluated over time in the dry season. ‘Pierrot’ was the most resistant and had the least colony forming units over time, followed by ‘Local Orange’. These can be considered as having some mechanism that prevents bacterial multiplication. ‘Champagne’ was the least resistant and had the most colony forming units over time.

There were large increases in CFUs with time following inoculation for the three cultivars; ‘Champagne’ had the largest increases and ‘Pierrot’ the smallest (Table 17). There was a strong linear regression (R²=0.99) between TLS and tissue bacterial concentration determined in three anthurium cultivars with varying levels of foliar resistance. Thus resistance at the foliar level may be due to varying levels of host resistance to bacterial multiplication.

TABLE 17
Number of colony forming units found in each inoculated disc per
cultivar over time (days after inoculation).

| | CFU/disc | | |
|---|---|---|---|
| Cultivar* | Day 5 | Day 7 | Day 10 |
| Champagne | 4.90 × 10⁶ | 1.41 × 10⁷ | 2.93 × 10⁸ |
| Local Orange | 2.91 × 10⁶ | 8.40 × 10⁶ | 1.92 × 10⁷ |
| Pierrot | 3.10 × 10⁵ | 7.70 × 10⁵ | 9.70 × 10⁵ |

*Cultivar differences for log₁₀ CFU/disc significant at P = 0.05.

Correlation Between Systemic and Foliar Bacterial Blight Resistance in Anthurium Andraeanum (Hort.)

Genetic correlations (Pearson's product moment correlation) between measures of systemic and foliar BB resistance are presented in Table 18. All correlation coefficients were small and not significant at P≦0.05, the highest value being between TLS (wet season) and proportion of dead plants (r=−0.33). This indicates that the two levels of resistance are different. This observation supports field observations, which also suggest that some cultivars that show high levels of foliar symptoms do not succumb to the disease at all.

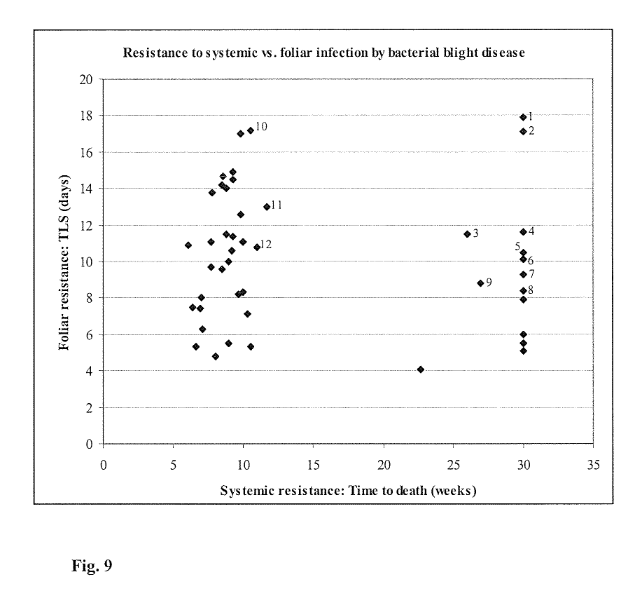

A scatter-plot of systemic resistance (time to death) vs. foliar resistance (TLS) (FIG. 9) showed that two anthurium cultivars (‘Acropolis’ and ‘Bianco’) were resistant to both phases of the disease; seven cultivars were tolerant to foliar resistance but resistant to systemic infection by bacterial blight disease (‘Venus’, ‘Local Pink’, ‘Cotopaxi’, ‘Mirjam’, ‘Tropical’, ‘Farao’, and ‘Senator’); two cultivars were resistant to bacterial blight disease at the foliar level and tolerant at the systemic level (‘Honduras’ and ‘Spirit’); and one cultivar was tolerant to both phases of the disease (‘Local Mina Pink’). Eight cultivars were resistant to foliar infection but susceptible to systemic infection by bacterial blight disease whereas the opposite was true for five cultivars. Six cultivars were susceptible to both phases of the bacterial blight disease.

TABLE 18
Genetic correlations between measures of systemic resistance (time to
death, proportion of dead plants) and foliar resistance (TLS) to
bacterial blight disease for 13 anthurium cultivars.
Pearson's Product Moment correlations
Foliar values

| | Systemic values | |
|---|---|---|
| | Time to death (days) | Proportion of dead plants |
| TLS^{a} wet season | −0.06 | −0.33 |
| TLS dry season | −0.07 | −0.04 |
| Mean TLS over seasons | −0.08 | −0.19 |

^{a}time taken for the lesions to entirely cover the leaf disc.

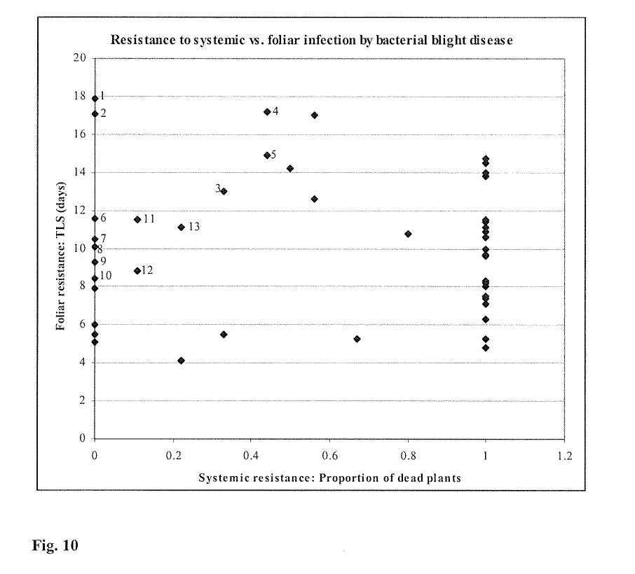

A scatter-plot of proportion of dead plants vs. TLS (FIG. 10) using 0.44 as the cut-off proportion point for systemic resistance (≦0.44) showed that ‘Acropolis’ and ‘Bianco’ were resistant to both phases; ‘Spirit’, ‘Honduras’, and ‘Terra’ were resistant to the foliar phase and highly tolerant to the systemic phase; ‘Local Pink,’ ‘Cotopaxi,’ ‘Mirjam,’ ‘Tropical,’ and ‘Farao’ were resistant to the systemic phase and tolerant to the foliar phase; and ‘Venus,’ ‘Senator,’ and ‘Local Whitish Pink’ were tolerant to both phases.

Mechanism of Resistance to Bacterial Blight Disease of Anthurium

The data provided herein demonstrate that foliar resistance to BB is mediated through the ability of the BB pathogen to multiply in the host tissue. This was clearly evident from the strong linear regression (R²=0.99) between TLS and tissue bacterial concentration determined in three anthurium cultivars with varying levels of foliar resistance. There was a tenfold difference in the bacterial multiplication rate between the susceptible and resistant cultivars of anthurium tested. Cultivar resistance to foliar invasion, measured as TLS, showed a continuous quantitative variation among the 50 cultivars evaluated for this level of resistance.

Artificial inoculation of cut petioles of anthurium plants in the experiments described supra resulted in various cultivar reactions, which fell into discrete categories. On the one hand there were cultivars that were highly resistant, which did not die at all, and did not show GFP fluorescence in any plant part (0% infection). The susceptible cultivars died rapidly, recording 100% plant death within 6 to 9 weeks of inoculation. There was an intermediate category where a varying proportion of anthurium plants died, but in a delayed fashion, taking between 10 to 27 weeks to die. The cultivars that died between 10 and 14 weeks clustered with the susceptible category, whereas the ones that took over 20 weeks to die clustered with the resistant category in a frequency distribution of 56 anthurium cultivars for time to death.

Screening for Resistance to the Bacterial Blight Disease in Anthurium

The differential system of susceptibility at the foliar and systemic levels requires screening methods capable of assessing resistance at the two levels, separately. In addition, a useful measure of resistance should be quantitative, be able to differentiate between various levels of resistance, provide repeatable results over trials and over seasons, and correlate to observed levels of field resistance. Furthermore, the method of screening developed should be amenable to genetic studies and for resistance breeding, where each progeny plant (genotype) resulting from hybridization is unique.

The methods of provided herein for evaluating foliar resistance generally use vacuum-infiltration, which aids in uniform lesion establishment in all leaf discs hence, reducing the experimental error and therefore improving the index of differentiation.

A standardized foliar screening method can be used based on the inoculation of leaf discs (5.6 cm²) from fully opened immature leaves (Stage 2) with an inoculum density of 10⁸ CFU/ml was developed to differentiate between cultivar resistances at the foliar level.

The best measure of resistance was the time taken for the lesion to cover the entire disc (TLS), which apart from having the highest index of differentiation, was the measure that best correlated to field resistance. TLS was also repeatable over trials within the wet season as evident from a comparison of regression lines, but increased in the dry season. The higher rate of pathogen invasion in the wet season can be attributed to higher temperatures and humidity.

The method provided herein is useful for screening foliar resistance to BB in large breeding populations, as each plant can be screened non-destructively based on replicate leaf discs from Stage 2 leaves. The method is straightforward and involves simply noting the number of days taken for leaf discs to completely deteriorate.

The methods described in the Examples used a modification of the petiole inoculation method of Fukui et al. (1998, supra) to assess systemic resistance of whole anthurium plants to the BB pathogen. The method used a bioengineered strain of X. campestris pv. dieffenbachiae containing a gfp-plasmid instead of the bioluminescent plasmid used by Fukui et al. (1996, supra). The GFP was easier to assay and was able to detect the presence of a few bacterial cells in the macerated leaf tissue, and hence, the method is amenable to large-scale screening. This is an improvement over the use of a bioluminescent Xanthomonas strain, which was difficult to detect in the tissue, especially at low concentrations and bacteria were found in regions where bioluminescence was not detected (Fukui et al., 1998, supra). Highly susceptible cultivars die within 6 to 10 weeks of inoculation, and the susceptible cultivars may go on for 14 weeks, and the rare infected plants belonging to the resistant group can remain alive for 20 to 27 weeks. The use of GFP allows the early detection of latent systemically infected plants, therefore reducing the time required to screen for resistance to BB in breeding programs.

Because there were no significant differences observed between the cultivars of ages of 1.5 to 5 years, the new methods can be effectively used to differentiate between levels of resistance among anthurium cultivars as early as 1.5 years. This can greatly reduce the length of the breeding cycles to incorporate resistance to systemic infection by BB and therefore improve the efficiency of breeding. Further, although there were no significant differences between the inoculum densities used with respect to the time taken to death, the higher inoculum density of 10⁹ CFU/ml eliminated the chance of escapes in susceptible cultivars, making the method more useful in screening segregating populations.

A comparison of the various measures of systemic resistance to BB showed that both proportion of infected/dead plants as well as the time taken to death were equally able to differentiate between the highly resistant and highly susceptible categories. These are characterized by 0% plant death and 100% plant death with a quick decline 6-10 WAI, respectively. Most cultivars fell into these two categories. A relatively small number of cultivars fell into two intermediate categories, a resistant category (with usually less than 30% plant death, but the decline was slow, taking 20 to 27 weeks for plants to die) and a tolerant category, with usually more than 50% infection with those infected dying within 12-13 WAI. The highly resistant and highly susceptible categories were highly repeatable over trials; however, the proportions fluctuated somewhat in the intermediary resistant and tolerant categories. Nevertheless, the time to death remained relatively constant, which suggested that the time taken to death might be a more repeatable measure in the intermediate categories. Furthermore, in screening a segregating population with single plant genotypes (each plant in the population is unique), the time taken to infection is the only practical measure. In such populations, the resistant and susceptible categories can be ascertained only if the plant gets infected but if it escapes infection then the plant will be erroneously placed into the highly resistant category. As the escapees are more likely to occur in the resistant/tolerant categories, this is not likely to affect the distribution of the categories if the segregating population is simply classified into resistant (including the highly resistant, resistant and tolerant categories) and susceptible categories. The frequency distribution of time to infection for the cultivars tested also showed that cultivars fell into two discrete categories, reflecting the above classification and therefore, provided further credence to this classification.

Identification of Resistance to Bacterial Blight Disease in Anthurium

The available parental cultivars were screened for resistance to BB at both foliar and systemic levels in the wet season, when the warm humid conditions provided for better symptom development. The relatively high percentage of resistance (approximately 25% at the foliar and 25% at the systemic levels) detected within the anthurium cultivars shows that the farmers' selection or natural selection of cultivars over the years has been reasonably successful. Routine epidemics during the past decade wiped out many of the highly susceptible category of anthurium cultivars in the country, which could not be saved using even the best management practices. This may account for the fact that no cultivar died prior to 6 WAI in this study It may also reflect the incorporation of BB resistance as one of the goals in anthurium breeding programs around the world in recent times.

Of the anthurium cultivars evaluated only ‘Acropolis’ and ‘Bianco’ combined resistance to both foliar and systemic infection of the disease, although there were approximately 25% of the cultivars that were resistant at either the foliar or systemic level. This shows that anthurium breeding programs to date have been inefficient in combining resistance to the disease at both levels. One reason for this may be the inadequate screening methods used to identify resistance in segregating populations.

Of the seven local anthurium cultivars screened for systemic resistance, four fell in the resistant to highly resistant category, two in the tolerant category and one in the susceptible category. This shows that there is variation within the local germplasm for systemic resistance to BB, with a large percentage falling in the resistant category. In contrast, the local cultivars fell into the moderately resistant or susceptible categories with respect to foliar resistance.

Although ‘Kalapana’ was referred to by Fukui et al. (1998, supra) as one that combines resistance to both foliar and systemic infection, in this study ‘Kalapana’ was among the most susceptible to foliar infection but was found to be resistant to systemic infection. This observation is also supported by field ranking for foliar resistance provided by anthurium growers in Trinidad, who also found ‘Kalapana’ to be highly susceptible to foliar symptoms of BB. This may be because of differences between the Hawaiian strain of X. campestris pv. dieffenbachiae and the local strain in Trinidad.

Resistance at both levels is necessary to protect the crop. Although systemic susceptibility causes death of plants and is the most economically important, foliar susceptibility can also affect the economic viability of the industry. Foliar symptoms are a major problem during the warmer and more humid wet season than in the dry season. Not only does it increase the cost of sanitation during the wet season, which often has to be done routinely, it also leads to loss of leaves and hence reduced productivity.

The studies described herein have been used to develop optimized screening methods useful for assessing resistance to BB at the foliar and systemic levels. The study also identified measures of resistance at both levels that were not only capable of differentiating quantitatively between different levels of resistance, but were repeatable and correlated to field resistance.

Assessment of resistance to systemic infection by BB can be carried out by injecting 100 μl of 10⁹ CFU/ml pathogen density into the cut-petiole of the second youngest leaf and measuring time to death and proportion of dead plants whereas, that for resistance to foliar infection can be done by inoculating leaf discs of stage 2 leaves with 10⁸ CFU/ml pathogen density and monitor the discs until disease lesions completely cover the discs.

Using these methods several promising cultivars of anthurium have been identified including ‘Acropolis’ and ‘Bianco’ that can serve as parents in breeding programs. Further, the quantitative assessment of resistance at both levels will allow the possibility of using quantitative genetic analysis methods to determine the genetic basis of resistance.

### Example 2

### Morpho-Physiological Characteristics Associated with Vase-Life in Anthurium Andraeanum (Hort.)

Although vase-life is considered an important horticultural quality character in anthurium, few studies have been conducted to determine the characteristics associated with variation in vase-life. Although post-harvest studies have pointed to dehydration and protein breakdown as possible mechanisms, this has not led to any practical selection criteria for improving vase-life in plant breeding programs. In addition, most studies in the past have used only a few cultivars.

The present invention relates to methods for assessing morpho-physiological characteristics of the anthurium cut-flower, determining their relationship to vase-life; and a method for determining vase-life based on the morpho-physiological characteristics evaluated.

Material and Methods

Location and Seasons

Experiments were conducted during the wet (September to October; October to January) and dry seasons (March to May) during 2001-2002 at the laboratories of the University of the West Indies, St. Augustine, Trinidad. Cut-flowers were collected from a commercial farm in Arima, Kairi Blooms Ltd, where they were grown under uniform cultural and management conditions.

Anthurium Cultivars

Overall, 26 anthurium cultivars (Table 19) were used in three experiments. The cultivars were selected to include various cut-flower shapes, textures, sizes and colors, and were all approximately the same age (3-4 years old). Two experiments were carried out in the dry and wet seasons using a similar set of cultivars (16 or 17) and a third was carried out using an entirely new set of cultivars (9) to test the prediction equation developed from the former experiments (Table 19).

Harvesting and Transport of Cut-Flowers

Cut-flowers with no scratches, no deformities and straight peduncles were harvested at the ¾ mature stage of the spadix (¾ of the true flowers were open on the spadix). Kamemoto (Kamemoto, 1962, Hawaii Farm Sci. 11(4):2-4) reported that maximum vase-life occurs when the inflorescences (spadix) are harvested with three-quarters or more of the true flowers open. The cut-flowers were cut with a sharp, sterile knife (sterilized by dipping in isopropanol alcohol), packed in boxes containing wet shredded paper as for export purposes and transported to the laboratory in an air-conditioned vehicle. Cut-flowers were harvested from along the length of the bed, for each cultivar, to ensure a good representation of the variation within each cultivar. Experiments were set up on the day of harvesting.

TABLE 19
Anthurium andraeanum (Hort.) cultivars evaluated in each vase-life
experiment

| Cultivars | Spathe color | Experiment used |
|---|---|---|
| Spirit | Pink | 1 |
| Lydia | Pink | 1, 2 |
| Local Pink | Pink | 1, 2 |
| Fantasia | White with pink veins | 1, 2 |
| Cuba | White | 1, 2 |
| Pierrot | White | 1, 2 |
| Acropolis | White | 1, 2 |
| Tropical | Red | 1, 2 |
| Honduras | Red and green | 1, 2 |
| Success | Red | 1, 2 |
| Mirjam | Red | 1, 2 |
| Midori | Green | 1, 2 |
| Tequila | Green | 1, 2 |
| Venus | Peach | 1, 2 |
| Fla Range | Orange | 1, 2 |
| Terra | Bronze | 1, 2 |
| Evergreen | White with green lobes | 1, 2 |
| Cheers | Peach | 3 |
| Lunette | Pink | 3 |
| Rosa | Pink | 3 |
| Champagne | White | 3 |
| Ibara | White | 3 |
| Gloria | Red | 3 |
| Sweety | Pink | 3 |
| Senator | Peach with green lobes | 3 |
| Kalapana | Red with green lobes | 3 |

Morpho-Physiological Characterization

The study to characterize morpho-physiological features of Anthurium cultivars was conducted during the dry season of 2001 (March to May). Nine cut-flowers per cultivar were harvested as previously described and subjected to morpho-physiological characterization.

Peduncle length was measured as the distance from the point of attachment of the peduncle to the spathe down to the cut-end using a measuring tape (Knight 3.6 meter Measuring Tape, Taiwan). Peduncle diameter was measured using a pair of calipers (ENKAY Vernier Caliper 5″ scale, No. 430-C) at a distance of 15 cm from the spathe.

Surface area of the spathe was obtained by tracing the spathe outline onto brown paper and measuring it with a ΔT Area Meter MK2 model (Delta-T Devices, Burwell Cambridge, England). The value obtained was doubled to find total spathe surface area. Spathe color was described using a commercial color wheel and using “farmer's” terminology. Spadix length was measured as the distance from the point of attachment of the inflorescence at the peduncle to the tip for ¾ ripe spadices (Dai and Paul, 1990, J. Am. Soc. for Horticul. Sci. 115(6):901-905).

Stomata density, stomata size, and hydathode density were determined on three randomly selected spathes per cultivar. Four 1.5×0.5 cm pieces were cut from each spathe and placed in a clearing solution in a petri dish. The clearing solution was prepared by adding 15 g of NaOH pellets to 500 ml of water and 500 ml of 95% ethanol. The clearing solution was replaced routinely as it became pigmented or dried up. Cleared sections were mounted on slides, stained with toluidine blue, and warmed for two minutes on a slide warmer at 40° C. Stomata density and hydathode density were enumerated for each section based on four views on the lower and four views on the upper spathe surface at ×100 magnification (Euromex, the Netherlands). From these measures, the mean number of stomata mm⁻² and mean number of hydathodes mm⁻² on the abaxial and adaxial spathe surfaces of each cultivar were calculated. Stomata size of each cultivar was measured under 400× magnification using a calibrated eyepiece micrometer (E1 (n)-19 mm, 01B19201, Pyser-SGI Ltd, UK). The length and width of ten randomly selected stomata from four views per section were measured and mean stomata length and width calculated. The length and width of stoma plus guard cell were also measured. From these data, the area of each stoma was calculated using the formula: area=πr₁r₂ where r₁ is half the length and r₂ is half the width; as the stomata were elliptical in shape.

Spathe thickness was recorded using three sections (1.5×0.5 cm sections along the midrib) per spathe for three spathes. The nine sections per cultivar were mounted in wax, cut cross-sectionally with a microtome (20 μm thicknesses) and fixed on slides based on the methods of Johansen (Johansen, 1940, Plant Microtechnique, London: McGraw Hill Book Company Inc.) and O′Brien and McCully (O′Brien and McCully, 1981, The study of plant Structure, principles land selected methods, Melbourne, Australia: Termarcarphi Pty. Ltd.). Slides were viewed under ×100 magnification and the mean thickness of each spathe was calculated by taking the average of five measurements per section using a calibrated eyepiece micrometer as before.

To determine epicuticular wax, three whole spathes were first traced on brown paper and then placed in phenol-chloroform until all the wax had been removed (about 40 seconds). The wax was dried in a rotovac (serial no. 103026, Haake Buehler Instruments Inc., 244 Saddle River Rd, Saddle Brook, N.J., USA) and weighed using an electronic balance (Acculab, AL-104, Pennsylvania, USA) to 0.0001 g accuracy. The traced surface area of the spathes was measured using the ‘leaf area meter’ and the average wax (mg/m²) was calculated.

Vase-Life Experiment 1

Seventeen cultivars, outlined in Table 19, were used in a vase-life experiment during March 2001 (the dry season). The cut-flowers were harvested and transported to the laboratory, as described supra. The experiment was conducted in sterile, 250 ml measuring cylinders in a laboratory setting (11 hours of white fluorescent light; 23.8° C.; 73.5% relative humidity) and arranged in a completely randomized design with 3-8 replications per cultivar. Cultivars ‘Pierrot’, ‘Mirjam,’ and ‘Fla Range’ had five replications each; ‘Venus’ had four replications, ‘Spirit’ had 7 replications, ‘Midori’ had 8 replications, and the others had three replications each. Each cylinder contained three cut-flowers placed in 210 ml of sterile distilled water. The cylinders were covered with a cellophane wrap to prevent evaporation. Before placing the cut-flowers into the cylinder, the outline of each spathe was traced on brown paper to determine the spathe area as described above, and the base of the peduncles were freshly cut under water at an angle of 45° using a sharp, sterile scalpel.

Cut-flowers were monitored until spathe or spadix deterioration with the following variables recorded daily: transpiration per cylinder; peduncle base browning, spadix browning/necrosis, spathe floppiness, spathe browning/necrosis, spathe discoloration, and loss of luster/glossiness in the spathes. Based on this study, a descriptive account of the deterioration process for each cultivar was developed.

Cylinders were topped to the original water level at the same time each day using a dispenser, and the amount of water required was noted as the transpiration value for that day. Five-day mean transpiration rates were calculated (per cut-flower basis) for each replicate of each of the 17 cultivars, for the study period. The experiment was continued for 70 days until all the cultivars began to show signs of deterioration. Data analysis is described in a subsequent section.

Vase-Life Experiment 2

The vase-life experiment described in above was repeated (Table 19) in the wet season (September-October 2001) using the same set of cultivars (except ‘Spirit’) under the same conditions. Three replicates with three cut-flowers per replicate were evaluated for each cultivar. The stomata densities in the adaxial and abaxial surfaces were measured as described above. Preliminary studies had demonstrated that the other morphological characteristics were invariable over seasons.

Vase-Life Experiment 3

A third vase-life experiment was carried out during the period October 2001 to January 2002 on an independent set of nine cultivars (Table 19) under the conditions described above. Each cultivar was replicated three times with three cut-flowers per replicate and arranged in a completely randomised design and monitored as described. The stomata densities in the adaxial and abaxial spathe surfaces were measured as described herein.

Data Analysis

One-way ANOVA was conducted using the NCSS statistical package (2001 version, Number Cruncher Statistical Systems, Kaysville, Utah, USA) to determine the significance of cultivar differences for the morpho-physiological characteristics studied including the five-day transpiration values and vase-life as determined by the time taken to deterioration. In the first experiment the daily five-day mean transpiration rate per cut-flower was measured up to 55 days after the initiation (DAI) of the experiment for each cultivar, and the significance of cultivar differences ascertained using ANOVA. The values were plotted separately for each cultivar on a time scale. The curve shapes were investigated using three variables; time at which inflection occurred on the curve, duration of steady state transpiration following the inflection on the curve, and the rate of steady state transpiration.

Pearson's product moment correlations (NCSS statistical package) were carried out to determine the correlation coefficients between morpho-physiological characteristics and vase-life. Multiple regression analysis (NCSS statistical package), and forward and backward model selections were performed to identify a minimal subset of characteristics that best predicted vase-life and time to spadix necrosis. For these purposes, spathe color of each cultivar was coded as follows: red/not red, orange/not orange, white/not white and green/not green as described by Kamemoto et al. (Kamemoto et al, 1988, U. Hawaii: Research Series 056, HITAHR, Coll. Tropical Agric. And Human Res. 8-88), and Kamemoto and Kuehnle (1996, Breeding Anthuriums in Hawaii, Honolulu, Hi., U. Hawaii Press). Boolean numbers 0 (absence of color) and 1 (presence of color) were used to describe categories.

In the second experiment the daily five-day mean transpiration rate per cut-flower was investigated up to 50 DAI for each cultivar, and the significance of cultivar differences ascertained using ANOVA as per vase-life Experiment 1. The values were plotted separately for each cultivar, and the shapes of the curves were investigated as per vase-life Experiment 1.

Pearson's product moment correlations (NCSS statistical package) were carried out to determine the correlation coefficients between morpho-physiological characteristics measured in vase-life Experiment 2 (five-day transpiration values, abaxial stomata density, time to spadix and spathe necrosis, days to steady state transpiration, duration and steady state transpiration) and vase-life.

Two-way ANOVA was carried out to test whether there was any significant genotype-environmental interaction (cultivar-season interaction) for abaxial stomata density, time to spadix and spathe necrosis, and vase-life for the 16 anthurium cultivars evaluated in Experiments 1 and 2. Comparison of regression lines (COLR program, Version 1, 1974, CARDI, St. Augustine, Trinidad) between average vase-life and vase-life in the wet or dry seasons was done to determine the stability of vase-life over seasons. Similarly, a comparison of regression lines for abaxial stomata density, time to spadix and spathe necrosis for wet and dry seasons was performed.

The regression equations developed in vase-life Experiment 1 to predict vase-life and time to spadix necrosis were validated in the second experiment (vase-life Experiment 2) using the same set of 16 cultivars and in the third experiment (vase-life Experiment 3) using a different set of 9 cultivars.

### Results

Vase-life Experiment 1

Average Transpiration Rate

There were significant differences (P<0.001) between cultivars for the five-day mean transpiration rate from 5 to 45 days (Table 20). The genotypic coefficient of variation increased steadily and reached a maximum value of 338% at 15 days and continued to steadily decrease thereafter. Cultivar differences for mean transpiration rate for days 50 and 55 were not significant (P>0.05).

The within cultivar variation in mean transpiration rates also increased as shown by the within cultivar CV (Table 3.2) suggesting that there were increasingly more replicate to replicate variation in behaviour. Nevertheless, there were some important and revealing trends in the mean transpiration rates over time.

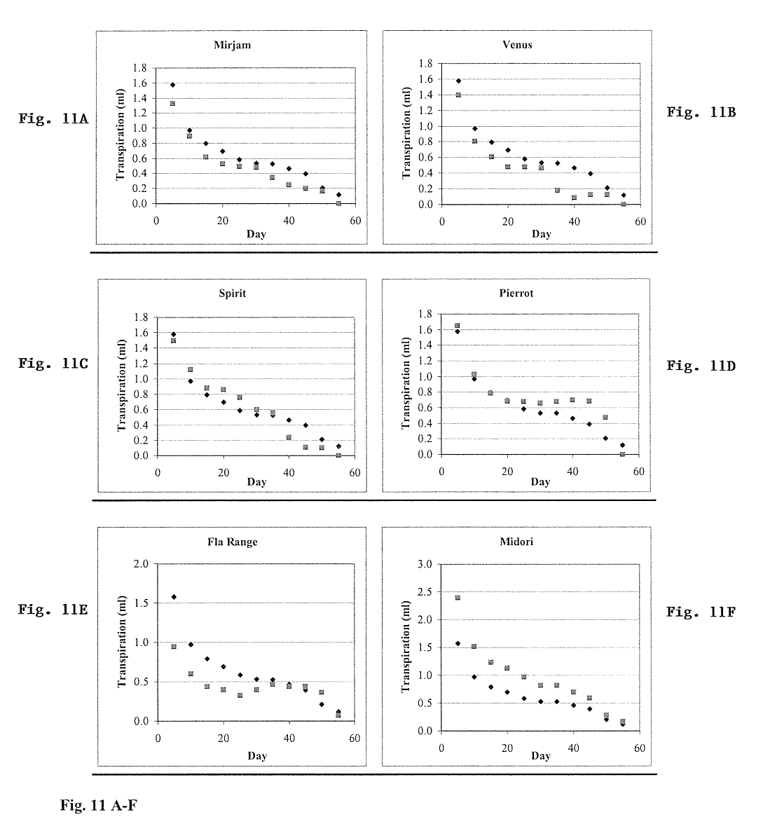

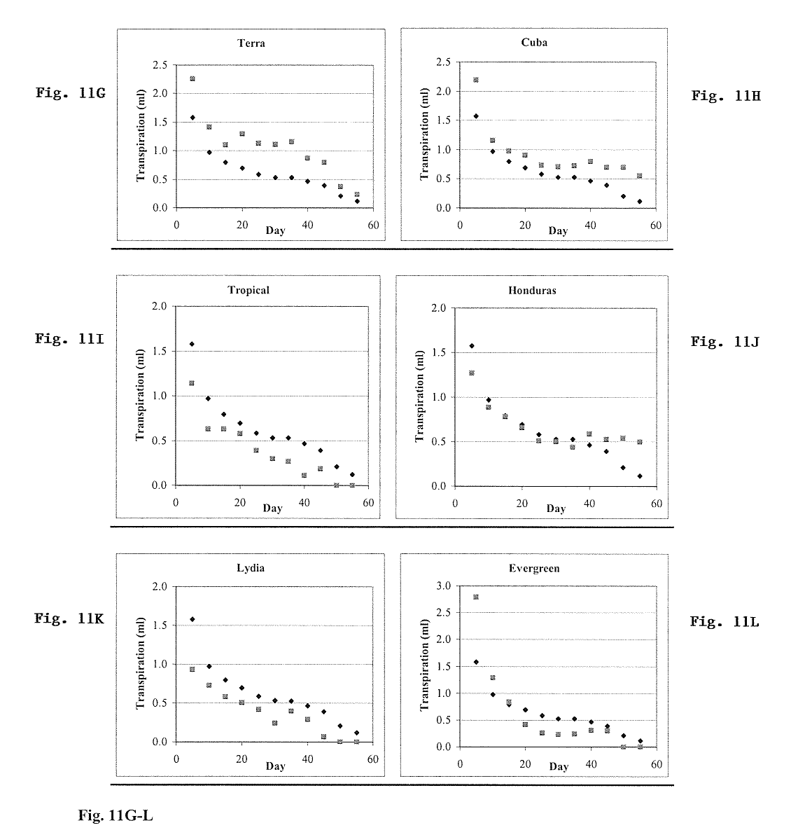

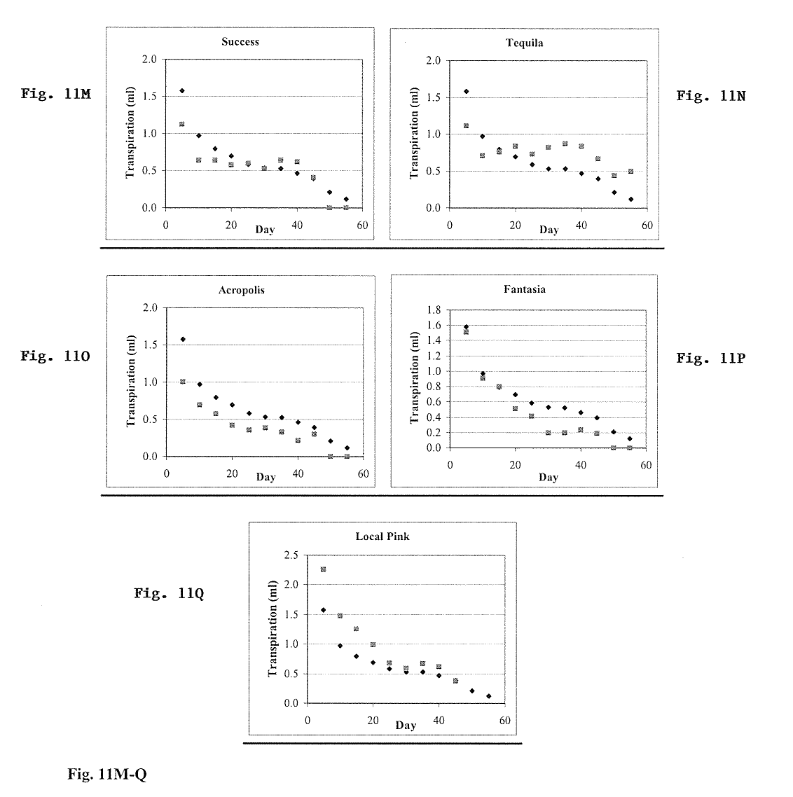

The mean daily transpiration rate over all cultivars (Table 20) showed a steady decline up to 25 days after initiation (DAI) but showed an inflection (FIG. 11), which maintained transpiration rates up to 40 DAI, but continued to decline rapidly thereafter. FIG. 11 and Table 21 show that the time at which the inflection occurred, the duration of inflection and the average steady state transpiration rates during the steady state period following the inflection varied widely with the anthurium cultivars. As a result the cultivars showing above average transpiration rates varied with time (Table 20). For instance, at 5 DAI, ‘Lydia’ (0.93 ml day⁻¹) and ‘Fla Range’ (0.95 ml day⁻¹) had the lowest daily average transpiration rates and ‘Evergreen’ (2.79 ml day⁻¹) the highest (Table 20). The average daily transpiration rate (5 DAI) for ‘Evergreen’ was more than three times that for ‘Lydia’ and ‘Fla Range’. By day 30, cultivar ‘Terra’ had the highest average transpiration rate (1.1 ml day⁻¹) and ‘Fantasia’, ‘Lydia’ and ‘Evergreen’ had the lowest (0.20-0.24 ml day⁻¹).

The results show that cut-flowers of some anthurium cultivars have a mechanism that allows them to maintain transpiration rates over a longer period of time and that there were differences between cultivars in this ability. FIG. 11A-FIG. 11Q [3.1] and Table 3.3 show that ‘Terra’, ‘Tequila’, ‘Cuba’, ‘Pierrot’, ‘Midori’, ‘Honduras’ and ‘Fla Range’ maintained a relatively higher transpiration rate for a longer time through this mechanism. This mechanism was not triggered until some level of decline in transpiration rate had occurred in the cultivars. There is some variation in the timing of this mechanism. Often the mechanism is triggered too late so that the cut-flowers would have already started showing desiccation symptoms.

Cut-Flower Senescence in Anthurium

The various indicators of cut-flower senescence in anthurium and the times at which they occur in various cultivars of anthurium are shown in Table 22. Some signs of senescence were common for all cultivars such as peduncle base browning, spadix and spathe necrosis, whereas others such

TABLE 20
Five-day average transpiration rate (ml day⁻¹) per cut-flower for 17
cultivars of Anthurium andraeanum (Hort.) in vase-life Experiment 1

| Cultivar | 5 | 10 | 15 | 20 | 25 | 30 | 35 | 40 | 45 | 50 | 55 |
|---|---|---|---|---|---|---|---|---|---|---|---|
| Mirjam | 1.33 | 0.90 | 0.62 | 0.53 | 0.49 | 0.48 | 0.35 | 0.25 | 0.20 | 0.17 | 0.00 |
| Venus | 1.40 | 0.81 | 0.61 | 0.48 | 0.48 | 0.47 | 0.18 | 0.09 | 0.13 | 0.13 | 0.00 |
| Spirit | 1.50 | 1.12 | 0.88 | 0.86 | 0.76 | 0.60 | 0.56 | 0.24 | 0.11 | 0.10 | 0.00 |
| Pierrot | 1.65 | 1.03 | 0.79 | 0.69 | 0.68 | 0.66 | 0.68 | 0.70 | 0.69 | 0.48 | 0.00 |
| Fla Range | 0.95 | 0.60 | 0.44 | 0.40 | 0.33 | 0.40 | 0.47 | 0.44 | 0.44 | 0.37 | 0.07 |
| Midori | 2.40 | 1.52 | 1.24 | 1.13 | 0.97 | 0.82 | 0.82 | 0.70 | 0.59 | 0.28 | 0.17 |
| Terra | 2.26 | 1.42 | 1.10 | 1.30 | 1.13 | 1.11 | 1.16 | 0.87 | 0.80 | 0.38 | 0.24 |
| Cuba | 2.19 | 1.16 | 0.98 | 0.91 | 0.74 | 0.71 | 0.73 | 0.80 | 0.70 | 0.70 | 0.56 |
| Tropical | 1.14 | 0.63 | 0.63 | 0.58 | 0.39 | 0.30 | 0.27 | 0.11 | 0.19 | 0.00 | 0.00 |
| Honduras | 1.27 | 0.89 | 0.78 | 0.66 | 0.51 | 0.50 | 0.44 | 0.59 | 0.53 | 0.54 | 0.50 |
| Lydia | 0.93 | 0.73 | 0.58 | 0.51 | 0.42 | 0.24 | 0.40 | 0.29 | 0.07 | 0.00 | 0.00 |
| Evergreen | 2.79 | 1.29 | 0.84 | 0.42 | 0.26 | 0.23 | 0.24 | 0.31 | 0.30 | 0.00 | 0.00 |
| Success | 1.13 | 0.64 | 0.64 | 0.58 | 0.60 | 0.53 | 0.64 | 0.62 | 0.41 | 0.00 | 0.00 |
| Tequila | 1.11 | 0.71 | 0.76 | 0.84 | 0.73 | 0.82 | 0.87 | 0.84 | 0.67 | 0.44 | 0.50 |
| Acropolis | 1.01 | 0.70 | 0.58 | 0.42 | 0.36 | 0.39 | 0.33 | 0.22 | 0.30 | 0.00 | 0.00 |
| Fantasia | 1.51 | 0.91 | 0.80 | 0.51 | 0.42 | 0.20 | 0.20 | 0.24 | 0.19 | 0.00 | 0.00 |
| Local Pink | 2.26 | 1.48 | 1.26 | 0.99 | 0.68 | 0.59 | 0.67 | 0.62 | 0.38 | 0.00 | 0.00 |
| Sig. level (P <) | 0.001 | 0.001 | 0.001 | 0.001 | 0.001 | 0.001 | 0.001 | 0.001 | 0.001 | (NS) | (NS) |
| Mean | 1.58 | 0.97 | 0.80 | 0.69 | 0.59 | 0.53 | 0.53 | 0.47 | 0.39 | 0.21 | 0.12 |
| SEM | 0.167 | 0.100 | 0.114 | 0.108 | 0.105 | 0.127 | 0.129 | 0.114 | 0.114 | 0.185 | 0.394 |
| LSD_{(0.05)} | 0.39 | 0.24 | 0.27 | 0.25 | 0.25 | 0.30 | 0.30 | 0.27 | 0.27 | — | — |
| CV within (%) | 0.20 | 0.21 | 0.29 | 0.30 | 0.34 | 0.46 | 0.48 | 0.47 | 0.57 | — | — |
| Genotypic CV (%) | 272 | 315 | 338 | 260 | 250 | 219 | 196 | 176 | 169 | 91 | 59 |

TABLE 21
Time to steady state transpiration and duration (days), transpiration
(ml) at steady state and vase-life of 17 Anthurium andraeanum
(Hort.) cultivars evaluated for vase-life during the dry season

| | | Duration of | | |
|---|---|---|---|---|
| | Time to inflection | steady state | Transpiration | |
| | on transpiration | transpiration | at steady | Vase-life |
| Cultivar | curve (days) | (days) | state (ml) | (days) |
| Mirjam | 15 | 15 | 0.53 | 18 |
| Venus | 20 | 10 | 0.48 | 15 |
| Spirit | 30 | 5 | 0.58 | 15 |
| Pierrot | 20 | 25 | 0.70 | 37 |
| Fla Range | 30 | 25 | 0.47 | 33 |
| Midori | 15 | 20 | 0.97 | 34 |
| Terra | 15 | 30 | 1.10 | 40 |
| Cuba | 15 | 35 | 0.80 | 49 |
| Tropical | 15 | 10 | 0.63 | 24 |
| Honduras | 25 | 30 | 0.55 | 45 |
| Lydia | 25 | 15 | 0.42 | 26 |
| Evergreen | 20 | 20 | 0.29 | 14 |
| Fantasia | 30 | 15 | 0.20 | 19 |
| Local pink | 25 | 15 | 0.62 | 17 |
| Acropolis | 20 | 25 | 0.33 | 18 |
| Tequila | 15 | 30 | 0.84 | 33 |
| Success | 20 | 20 | 0.59 | 16 |

TABLE 22
Mean time (days) to cut-flower senescence of Anthurium andraeanum
(Hort.) during vase-life Experiment 1 (dry season) based on six
physiological processes
Results for vase-life Experiment 1

| | Mean days to: | | | | | | |
|---|---|---|---|---|---|---|---|
| | Ped. | | Loss of | | | | |
| | base | | Luster | Spadix | | Spathe | Vase- |
| Cultivar | brown^{b} | Flop.^{c} | (Spathe) | necr.^{d} | Spathe disc.^{e} | necr. | life |
| Spirit | 8 | 15 | 18 | 19 | — | 31 | 15 |
| Lydia | 9 | —a | —a | 26 | — | 26 | 26 |
| Local Pink | 8 | —a | 20 | 17 | 20 (light pinking) | 30 | 17 |
| Fantasia | 14 | —a | 20 | 19 | 20 (light pinking) | 22 | 19 |
| Cuba | 8 | —a | —a | 49 | 54 (greening) | — | 49 |
| Pierrot | 15 | —a | —a | 37 | — | 52 | 37 |
| Acropolis | 10 | 23 | —a | 18 | — | 24 | 18 |
| Tropical | 12 | —a | —a | 24 | 26 (blueing) | 27 | 24 |
| Honduras | 8 | —a | —a | 45 | 56 (blueing) | 61 | 45 |
| Success | 9 | 27 | 25 | 16 | 29 (blueing) | 33 | 16 |
| Mirjam | 12 | —a | 18 | 18 | 19 (blueing) | 23 | 18 |
| Midori | 12 | —a | —a | 34 | 40 (light greening) | 54 | 34 |
| Tequila | 8 | —a | —a | 33 | 34 (bleaching) | 38 | 33 |
| Venus | 12 | —a | 15 | 23 | 16 (bleaching/blueing) | 26 | 15 |
| Fla Range | 12 | —a | —a | 33 | 39 (bleaching/blueing) | 45 | 33 |
| Terra | 15 | —a | 42 | 40 | 42 (pinking) | 47 | 40 |
| Evergreen | 8 | 14 | —a | 18 | — | 19 | 14 |
| Significance | 0.001 | | | 0.001 | | 0.001 | 0.001 |
| (P≦) | | | | | | | |
| Mean | 10.0 | | | 27.6 | | 34.9 | 26.6 |
| SEM | 0.62 | | | 2.20 | | 2.70 | 2.19 |
| LSD (0.05) | 1.47 | | | 5.3 | | 6.32 | 5.21 |
| Genotypic CV | 24 | | | 39 | | 37 | 43 |
| (%) | | | | | | | |
| ID^{f} | 2.2 | | | 2.5 | | 1.6 | 2.7 |

‘—a’ physiological change was not observed throughout the course of the experiment.
^{b}Peduncle base browning;
^{c}Spathe floppiness;
^{d}necrosis;
^{e}discoloration;
^{f}= Index of differentiation.

as spathe floppiness, spathe discoloration, loss of glossiness or lustre were observable only in some cultivars. Loss of lustre (7/17) was more common among cultivars than floppiness (4/14). Spathe discoloration occurred in all cultivars except five (‘Spirit’ ‘Lydia’ ‘Pierrot’ ‘Acropolis’, and ‘Evergreen’).

Spathe discoloration can take the form of fading/bleaching, blueing or greening. Blueing was peculiar to all red cultivars. Any one of the indicators of senescence that affects the spathe or spadix appearance can result in the loss of vase-life. Obviously the symptom of senescence that occurs first determines the vase-life of the cut-flower, and hence the need for a complex method of vase-life assessment in anthurium.

Peduncle base browning is usually the first sign of senescence in anthurium cut-flowers. This occurred between 8-15 days in the various cultivars, with approximately half showing browning in 8-10 days, another third between 11-13 days and only a small fraction (‘Fantasia’, ‘Pierrot’ and ‘Terra’) later. This is usually followed by spadix necrosis in all anthurium cultivars. However, some anthurium cultivars (e.g., ‘Spirit’, ‘Mirjam’ and ‘Venus’) may show spathe floppiness or loss of lustre a few days earlier, or later. Spadix necrosis is often followed by spathe discoloration and then by spathe necrosis. The time course for these senescence symptoms, however, varies with the cultivars.

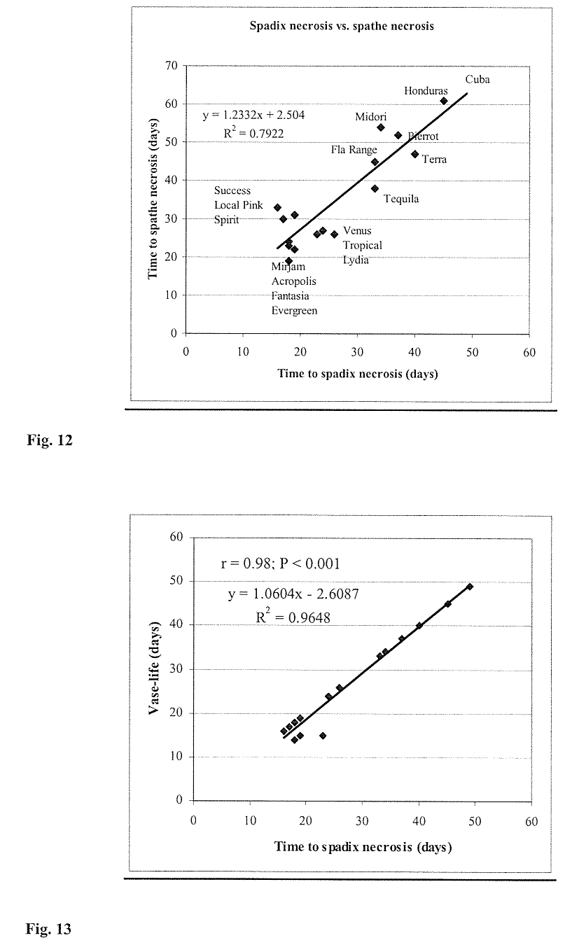

There were significant differences (P<0.001) between cultivars for peduncle base browning, but this senescence symptom did not correlate significantly with vase-life (r=0.20). There was a strong and significant linear correlation between time to spadix necrosis and spathe necrosis (r=0.88; P<0.001; FIG. 12); time to spadix necrosis and time to spathe discoloration (r=0.91; P<0.001) and between time to spathe necrosis and time to spathe discoloration (r=0.95; P<0.001). From FIG. 12, one can glean that ‘Honduras’, ‘Pierrot’, ‘Midori’, ‘Terra’, ‘Fla Range’ and ‘Tequila’ had delayed senescence, as indicated by time to spadix and spathe necrosis.

In 82% of cultivars, the loss of vase-life was attributed to spadix necrosis (Table 22), which was the first observable senescence symptom affecting cut-flower quality. In two (12%) cultivars (‘Spirit’ and ‘Evergreen’) loss of vase-life was due to spathe floppiness and in one cultivar (‘Venus’) it was due to loss of lustre, but was immediately followed by spadix necrosis. As a result, spadix necrosis was highly correlated (r=0.98) to vase-life, determined by a combined criterion (FIG. 13 [3.3]). The results show that spadix necrosis is a generally acceptable criterion to measure vase-life of anthurium cultivars.

There was a wide variation in the vase-life of cultivars (Table 3.4). Vase-life, determined by time to spadix necrosis, varied from 14 to 49 days (range of 35 days; 3.5 fold variation) among the anthurium cultivars investigated. ‘Evergreen’ and ‘Spirit’ had the shortest and second shortest vase-life, while ‘Cuba’ and ‘Honduras’ had the longest and second longest, respectively.

Morpho-Physiological Characteristics

The morpho-physiological data collected from the 17 cultivars used in vase-life Experiment 1 are presented in Table 3.5. There were significant differences between cultivars for all characteristics measured except adaxial spathe hydathode density (Table 23). Differences between abaxial spathe hydathode density and epicuticular wax content were significant at P≦0.05 whereas differences for other characteristics were highly significant at P≦0.001. Large genotypic coefficients of variation were recorded for adaxial and abaxial stomata density as well as abaxial hydathode density.

Abaxial stomata density ranged from 1.82 per mm² in the cultivar ‘Fla Range’ to 25.70 per mm² in the cultivar ‘Evergreen’, with a mean of 6.18 per mm² The frequency distribution for abaxial stomata density was skewed to the right. Only three cultivars (‘Fantasia’, ‘Acropolis’ and ‘Evergreen’) had abaxial stomata density greater than 10 per mm² and four between 5 and 10, whereas most (10 of the 17 cultivars or 60%) had stomata densities of five and below. Adaxial stomata densities were very small in all cultivars. The cultivar, ‘Cuba’ had no adaxial stomata.

Stomata size (Table 23) showed an approximately two-fold variation (209-442 μm²) among cultivars, with ‘Midori’ having the smallest size and ‘Success’, the largest stomata size. The mean stomata size was 331 μm².

Abaxial hydathode (Table 23) densities for all cultivars were very low and varied between 0-270 per cm². The highest value observed was for ‘Evergreen’, while ‘Pierrot’ had none. The abaxial hydathode densities were on average 60-fold lower than abaxial stomata density. Adaxial hydathode densities were also very small in all cultivars, with 53% of cultivars having no adaxial hydathodes. The large standard error for hydathode density supports the uneven distribution noted in observations under the microscope. This suggests that hydathode density may require a much larger sample size and/or sampling area to get good estimates.

TABLE 23
Morpho-physiological characteristic of 17 Anthurium andraeanum
(Hort.) cultivars grown in Trinidad (vase-life Experiment 1)

| | Spathe | | | | | | | | | | | | |
|---|---|---|---|---|---|---|---|---|---|---|---|---|---|
| | Stomatal | | Hydathode | | | Epicuticular | | | | | | | |
| | Number Per | | Number Per | | | Wax | Surface | | Spadix | | Peduncle | | Vase- |
| | mm² | | mm² | | Stomatal | Content | area | Thickness | Length | Diameter | Diameter | Length | life |
| Cultivar | Adaxial | Abaxial | Adaxial | Abaxial | Size (μm²) | (mg/m²) | (cm²) | (mm) | (mm) | (mm) | (mm) | (cm) | (days) |
| Spirit | 0.1 | 9.6 | 0.00 | 0.1 | 266.0 | 50 | 302.0 | 0.39 | 70 | 8 | 5.3 | 49.6 | 15 |
| Lydia | 0.0 | 2.8 | 0.00 | 0.0 | 324.8 | 130 | 306.0 | 0.41 | 74 | 9 | 5.7 | 56.0 | 26 |
| Local Pink | 0.1 | 2.2 | 0.04 | 0.1 | 380.5 | 160 | 213.2 | 0.47 | 69 | 9 | 6.1 | 63.7 | 17 |
| Fantasia | 0.0 | 16.4 | 0.02 | 0.2 | 220.8 | 80 | 269.8 | 0.46 | 73 | 10 | 6.7 | 45.3 | 19 |
| Cuba | 0.0 | 1.9 | 0.02 | 0.0 | 376.1 | 70 | 301.4 | 0.49 | 84 | 10 | 5.7 | 47.0 | 49 |
| Pierrot | 0.2 | 5.0 | 0.02 | 0.0 | 442.4 | 60 | 271.8 | 0.47 | 80 | 9 | 6.1 | 56.4 | 37 |
| Acropolis | 0.0 | 11.0 | 0.02 | 0.1 | 210.3 | 150 | 210.6 | 0.41 | 77 | 8 | 4.9 | 35.9 | 18 |
| Tropical | 0.1 | 3.3 | 0.00 | 0.0 | 352.3 | 90 | 322.8 | 0.43 | 67 | 10 | 6.2 | 52.6 | 24 |
| Honduras | 0.1 | 4.2 | 0.00 | 0.0 | 402.6 | 140 | 286.4 | 0.39 | 60 | 10 | 5.7 | 48.8 | 45 |
| Success | 0.0 | 2.6 | 0.00 | 0.0 | 447.8 | 100 | 250.6 | 0.54 | 78 | 10 | 6.0 | 45.4 | 16 |
| Mirjam | 0.2 | 6.6 | 0.04 | 0.1 | 248.3 | 170 | 221.6 | 0.48 | 53 | 10 | 5.1 | 48.3 | 18 |
| Midori | 0.0 | 5.9 | 0.02 | 0.1 | 209.4 | 120 | 372.8 | 0.47 | 59 | 9 | 5.7 | 54.3 | 34 |
| Tequila | 0.1 | 4.1 | 0.00 | 0.0 | 255.7 | 100 | 260.6 | 0.52 | 64 | 10 | 6.0 | 56.8 | 33 |
| Venus | 0.1 | 5.3 | 0.00 | 0.2 | 358.1 | 70 | 328.0 | 0.53 | 69 | 9 | 5.3 | 42.7 | 15 |
| Fla Range | 0.1 | 1.8 | 0.00 | 0.3 | 399.2 | 120 | 190.4 | 0.56 | 67 | 9 | 5.9 | 54.1 | 33 |
| Terra | 0.2 | 5.1 | 0.04 | 0.0 | 398.9 | 90 | 300.2 | 0.44 | 68 | 11 | 5.8 | 52.5 | 40 |
| Evergreen | 0.0 | 25.7 | 0.00 | 0.4 | 328.5 | 70 | 804.4 | 0.34 | 69 | 11 | 7.0 | 40.5 | 14 |
| ^{b}Sig. | 0.001 | 0.001 | NS^{a} | 0.05 | 0.001 | 0.05 | 0.001 | 0.001 | 0.001 | 0.001 | 0.001 | 0.001 | 0.001 |
| (P ≦) | | | | | | | | | | | | | |
| Mean | 0.07 | 6.7 | 0.01 | 0.1 | 330.7 | 104.1 | 306.6 | 0.459 | 69.5 | 9.5 | 5.8 | 50 | 26.6 |
| SEM | 0.04 | 1.05 | 0.02 | 0.08 | 28.34 | 0.02 | 24.39 | 0.02 | 2.35 | 0.4 | 0.25 | 2.58 | 2.29 |
| LSD_{(0.05)} | 0.1 | 3.01 | — | 0.23 | 78.56 | 0.07 | 69.34 | 0.06 | 5.52 | 0.93 | 0.71 | 7.32 | 5.21 |
| Genotypic | 83.1 | 92.7 | — | 108.2 | 24.4 | 35.1 | 44.6 | 13 | 11.4 | 9.2 | 9.1 | 13.8 | 43.1 |
| CV (%) | | | | | | | | | | | | | |
| ID^{c} | 0.92 | 3.43 | | 0.70 | 0.90 | 0.90 | 3.19 | 1.86 | 1.04 | 0.71 | 1.30 | 1.53 | 2.70 |

^{b}Significance;
‘NS^{a}’ cultivar differences not significant;
^{c}index of differentiation.

Epicuticular wax content varied from 50 mg m⁻² in ‘Spirit’ to 170 mg m⁻² in ‘Mirjam’ (Table 23), with a 3.5-fold variation among cultivars. There was a large variation between cultivars for this characteristic.

The cultivar, ‘Fla Range’ had the smallest spathe surface area (190 cm²) and ‘Evergreen’, the largest (804 cm²). The spathe size of ‘Evergreen’ was more than twice that of ‘Midori’, the second largest spathe among the cultivars studied (Table 23). Similarly ‘Evergreen’ had the narrowest spathe, whereas ‘Fla Range’ and ‘Success’ had the broadest (Table 23).

Spadix length varied from 53 mm in the cultivar ‘Mirjam’ to 84 mm in ‘Cuba’ with a general mean of 69.5 mm. ‘Spirit’ and ‘Acropolis’ had the narrowest spadix (8 mm) while ‘Terra’ and ‘Evergreen’ had the widest (Table 23).

‘Evergreen’ had the widest peduncle diameter, whereas the cultivar ‘Acropolis’ had the narrowest (Table 23). ‘Acropolis’ also had the shortest peduncle, whereas ‘Local Pink’ had the longest (Table 23).

Character Correlation and Regression Analysis

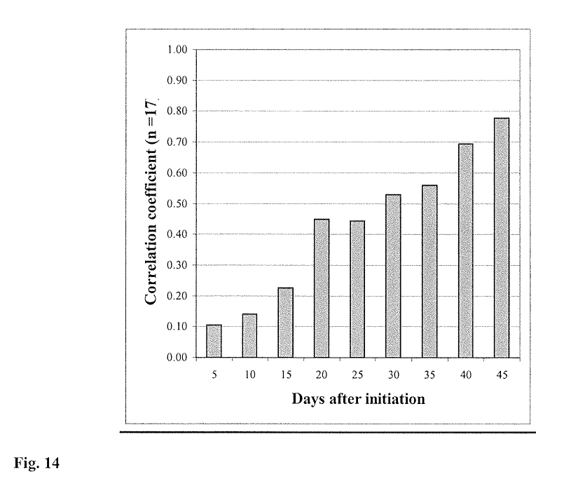

Correlation analysis between vase-life and average daily transpiration rates at 5, 10, 15, 20, 25, 30, 35, 40, and 45 DAI (FIG. 14) showed that the correlation coefficients steadily increased from 0.11 to 0.78. The results show that the ability of an anthurium cultivar to maintain transpiration rates later in the life of the cut-flower determines its vase-life.

The correlation between abaxial stomata density and transpiration at day 10 (r=0.51), day-15 (r=0.52) DAI were significant (P≦0.05). Spadix length and transpiration rate at 30 (r=0.51), 45 (r=0.49) and 50 (r=0.55) DAI showed significant correlation (P≦0.05). Peduncle length correlated significantly (P≦0.05) with transpiration rate at 40(r=−0.51) and 45 (r=−0.52) DAI (Table 3.6). Average transpiration (over 5-50 DAI) had significant positive correlation (P≦0.05) with abaxial stomatal density (r=0.50) and spathe surface area, r=0.60 (Table 24).

Pearson's product moment correlation between vase-life and components of average daily transpiration curve (Table 25) showed that the correlation coefficients were moderately large and significant for duration of steady state transpiration (r=0.68; P≦0.01). Duration of steady state transpiration alone explained 46% of the variation in vase-life. Days to steady state transpiration and steady transpiration rates were correlated (r=−0.55; P≦0.05). Steady rate transpiration explained 17% of the variation in vase-life.

TABLE 24
Genetic correlation between transpiration and other morpho-
physiological characteristics of Anthurium andraeanum (Hort.) in
Experiment 1 (dry season)
Pearson's product moment correlation

| | Mean transpiration rate at | | | | | | | | | | |
|---|---|---|---|---|---|---|---|---|---|---|---|
| | | | | | | | | | | | Mean |
| Character | Day 5 | Day 10 | Day 15 | Day 20 | Day 25 | Day 30 | Day 35 | Day 40 | Day 45 | Day 50 | Day 5-50 |
| Sto ad^{a} | −0.09 | 0.00 | 0.03 | −0.08 | −0.09 | −0.33 | −0.25 | −0.28 | −0.40 | −0.35 | 0.08 |
| Sto ab^{b} | 0.39 | 0.51* | 0.52* | 0.38 | 0.29 | 0.25 | 0.32 | 0.33 | 0.21 | −0.07 | 0.50* |
| Hyd ad^{c} | 0.08 | 0.17 | 0.06 | 0.02 | 0.04 | −0.18 | −0.04 | −0.17 | −0.26 | −0.21 | 0.38 |
| Hyd ab^{d} | 0.06 | 0.21 | 0.26 | 0.15 | 0.07 | 0.11 | 0.17 | 0.14 | 0.04 | −0.20 | 0.40 |
| Sto size^{e} | −0.25 | −0.15 | 0.04 | 0.01 | −0.06 | −0.10 | −0.18 | −0.14 | −0.08 | 0.11 | −0.08 |
| Wax^{f} | −0.12 | −0.11 | −0.17 | −0.05 | −0.06 | −0.17 | −0.15 | −0.38 | −0.39 | −0.34 | −0.17 |
| Sp_area^{g} | 0.40 | 0.41 | 0.47 | 0.18 | −0.01 | −0.01 | 0.04 | 0.15 | 0.01 | −0.17 | 0.60** |
| Sp_thick^{h} | −0.41 | −0.44 | −0.39 | −0.29 | −0.18 | −0.08 | 0.00 | 0.12 | 0.14 | 0.16 | −0.37 |
| Spd_lenⁱ | 0.08 | 0.21 | 0.20 | 0.33 | 0.40 | 0.51* | 0.36 | 0.37 | 0.49* | 0.55* | 0.39 |
| Spd-dia^{j} | −0.12 | −0.14 | 0.02 | −0.20 | −0.31 | −0.43 | −0.24 | −0.01 | −0.12 | −0.11 | −0.20 |
| Ped_dia^{k} | 0.27 | 0.31 | 0.39 | 0.11 | 0.04 | −0.01 | 0.00 | 0.15 | 0.12 | 0.06 | 0.38 |
| Ped_len^{l} | −0.05 | −0.12 | −0.19 | −0.34 | −0.25 | −0.37 | −0.46 | −0.51* | −0.52* | −0.34 | 0.08 |
| Vase-life | −0.10 | −0.30 | −0.31 | −0.41 | −0.42 | −0.44 | −0.38 | −0.34 | −0.18 | −0.27 | −0.24 |

^{a}Adaxial stomata;
^{b}Abaxial stomata;
^{c}Adaxial hydathode;
^{d}Abaxial hydathode;
^{e}Stomata size;
^{f}Spathe epicuticular wax content;
^{g}Spathe surface area;
^{h}Spathe thickness;
ⁱspadix length;
^{j}Spadix diameter;
^{k}Peduncle diameter;
^{l}Peduncle length.
*, **Correlation coefficients are significant at P ≦ 0.05 and P ≦ 0.01 and 15 d.f. when r = 0.482 and 0.606. respectively.

TABLE 25
Genetic correlation between transpiration parameters and vase-life of
17 Anthurium andraeanum (Hort.) cultivars evaluated in vase-life
Experiment 1 (dry season)
Pearson Correlations Section (Pair-Wise Deletion)

| | Duration of | | |
|---|---|---|---|
| | steady state | Transpiration | |
| | transpiration | at steady state | Vase-life |
| Onset of | −0.09 | −0.55 | 0.02 |
| steady state | | | |
| (DAI) | | | |
| | Duration of | 0.18 | 0.68 |
| | steady state | | |
| | transpiration | | |
| | | Transpiration | 0.41 |
| | | at steady state | |

Correlation (Table 26) between morphological characteristics and components of the mean daily transpiration curve showed that steady state transpiration values had significant correlation with abaxial, adaxial stomata density (r=−0.51, P≦0.05; r=0.54, P≦0.05, respectively) and abaxial hydathode density (r=−0.59; P≦0.05). Although not significant, peduncle length and vase-life showed positive trends whereas white/not white showed negative trends with vase-life. Days to steady state transpiration had significant (P≦0.05) correlation with white/not white (r=0.51), and showed negative trends with adaxial stomata density. Duration of steady state transpiration had significant (P≦0.05) correlation with red/not red (r=−0.58) and green/not green (r=0.49).

Pearson's correlations between morpho-physiological characteristics are presented in Table 27. There was significant positive correlation (P≦0.01) between abaxial hydathode and stomata density (r=0.73). Spathe surface area had significant correlation (P≦0.01) with abaxial stomata density (r=0.74) and abaxial hydathode density (r=0.63).

There was significant correlation (P≦0.05) between spathe thickness and abaxial stomata density (r=−0.59), and between spathe thickness and spathe surface area (r=−0.56). Therefore generally, cultivars with thicker spathes tended to have lower abaxial stomata density and smaller spathe surface area.

There was significant correlation (P≦0.05) between peduncle diameter and spathe surface area (r=0.55). Thus cultivars with larger spathe sizes generally had wider peduncles. Peduncle length and abaxial stomata density had significant (P≦0.05) negative correlation, r=−0.57. Therefore, cultivars with longer peduncles generally had a lower abaxial stomata density.

Spadix diameter had significant correlation (P≦0.05) with white/not white (r=0.60) indicating that cultivars with white spathes had wider spadices. Spadix length had significant correlation (P≦0.05) with green/not green (r=0.51) and peduncle diameter (r=0.58). Spadix green/not green (P≦0.01) had significant correlation with spathe green/not green (r=0.61) and spadix length (r=0.73). It showed negative trends with spadix diameter (r=−0.43) and positive trends with vase-life (r=0.38), although the correlations were not significant. Green/not green also had significant correlation (P≦0.05) with spathe surface area (r=0.48).

TABLE 26
Genetic correlation between cut-flower characteristics and
transpiration for 16 Anthurium andraeanum (Hort.) cultivars
evaluated in vase-life Experiment 1 (dry season)

| | | Duration | |
|---|---|---|---|
| | Days to steady | of steady | Steady state |
| Character | state ^{a}transp. | state transp. | transp. |
| Red/not red | 0.12 | −0.58 | −0.17 |
| Orange/not orange | −0.30 | 0.23 | 0.15 |
| White/not white | 0.51 | 0.17 | −0.40 |
| Green/not green | 0.00 | 0.49 | 0.26 |
| Adaxial stomata number | −0.43 | −0.06 | 0.48 |
| Abaxial stomata number | 0.38 | −0.20 | −0.50 |
| Adaxial hydathode number | −0.23 | −0.01 | 0.31 |
| Abaxial hydathode number | 0.25 | −0.12 | −0.59 |
| Stomata size | 0.17 | 0.34 | 0.19 |
| Spathe epicuticular wax | 0.15 | 0.05 | −0.17 |
| Spathe surface area | 0.22 | −0.12 | −0.25 |
| Spathe thickness | −0.29 | 0.32 | 0.14 |
| Peduncle diameter | 0.25 | 0.13 | −0.29 |
| Peduncle length | −0.15 | 0.05 | 0.45 |
| Spadix diameter | 0.17 | 0.15 | −0.05 |
| Spadix length | 0.07 | 0.29 | 0.01 |
| Vase-life | 0.02 | 0.68 | 0.41 |

^{a}Transp. = Transpiration.
The cultivar, ‘Midori’, was not used because it did not have a steady phase of transpiration.

TABLE 27
Genetic correlation between morpho-physiological characteristics
measured for 17 Anthurium andraeanum (Hort.) cultivars grown in
Trinidad during the dry season (vase-life Experiment 1)
Pearson's product moment correlation coefficient

| | Stom. | Stom. | Hyd. | Hyd. | Stom. | Spathe | Spathe | Spathe | Ped. | Ped. | Spad. | Spad. | Vase- |
|---|---|---|---|---|---|---|---|---|---|---|---|---|---|
| | Ad. | Ab. | Ad. | Ab. | Size | Wax | S. area | Thick. | Dia. | Length | Dia. | Leng. | life |
| Rd/Nr^{a} | 0.02 | −0.11 | −0.03 | −0.20 | 0.00 | 0.29 | −0.25 | −0.20 | −0.03 | 0.17 | −0.18 | −0.03 | −0.35 |
| Or/Nor^{b} | 0.30 | −0.20 | 0.01 | 0.25 | 0.32 | −0.14 | −0.12 | 0.41 | −0.15 | −0.02 | −0.09 | 0.07 | 0.11 |
| Wh/Nwh^{c} | −0.29 | 0.57* | 0.13 | 0.26 | −0.12 | −0.33 | 0.32 | −0.28 | 0.31 | −0.48* | 0.60** | 0.05 | 0.04 |
| Gr/Ngr^{d} | 0.06 | 0.25 | −0.04 | 0.08 | −0.10 | 0.00 | 0.48* | −0.30 | 0.26 | 0.06 | −0.46 | 0.51* | 0.38 |
| | Stom. | −0.14 | 0.42 | −0.14 | 0.21 | 0.01 | −0.18 | 0.00 | −0.23 | 0.27 | −0.38 | 0.11 | 0.09 |
| | Ad.^{e} | | | | | | | | | | | | |
| | | Stom. | −0.07 | 0.73** | −0.42 | −0.27 | 0.74** | −0.59* | 0.44 | −0.57* | −0.01 | 0.23 | −0.45 |
| | | Ab.^{f} | | | | | | | | | | | |
| | | | Hyd. | −0.19 | −0.12 | 0.38 | −0.28 | 0.04 | −0.16 | 0.21 | −0.11 | 0.11 | 0.10 |
| | | | Ad.^{g} | | | | | | | | | | |
| | | | | Hyd. | −0.15 | −0.16 | 0.63 | −0.17 | 0.44 | −0.36 | −0.14 | 0.17 | −0.47 |
| | | | | Ab.^{h} | | | | | | | | | |
| | | | | | Stom. | −0.19 | −0.04 | 0.23 | 0.23 | 0.26 | 0.34 | 0.27 | 0.32 |
| | | | | | Sizeⁱ | | | | | | | | |
| | | | | | | Spathe | −0.39 | 0.05 | −0.36 | 0.17 | −0.47 | −0.13 | −0.05 |
| | | | | | | Wax^{j} | | | | | | | |
| | | | | | | | Spathe | −0.56* | 0.55* | −0.32 | −0.03 | 0.44 | −0.19 |
| | | | | | | | S. area^{k} | | | | | | |
| | | | | | | | | Spathe | −0.14 | 0.24 | 0.05 | −0.07 | 0.11 |
| | | | | | | | | Thick.^{l} | | | | | |
| | | | | | | | | | Ped. | 0.16 | 0.15 | 0.58* | −0.02 |
| | | | | | | | | | Dia.^{m} | | | | |
| | | | | | | | | | | Ped. | −0.20 | −0.06 | 0.31 |
| | | | | | | | | | | Lengthⁿ | | | |
| | | | | | | | | | | | Spad. | −0.17 | 0.06 |
| | | | | | | | | | | | Dia.^{o} | | |
| | | | | | | | | | | | | Spad. | 0.24 |
| | | | | | | | | | | | | leng^{p} | |

^{a}Red/not red;
^{b}Orange/not orange;
^{c}White/not white;
^{d}Green/not green;
^{e}adaxial stomata density;
^{f}abaxial stomata density;
^{g}Adaxial hydathode density;
^{h}Abaxial stomata density;
ⁱstomata size;
^{j}Spathe epicuticular wax content;
^{k}Spathe surface area;
^{l}Spathe thickness;
^{m}Peduncle diameter;
ⁿPeduncle length;
^{o}Spadix diameter;
^{p}Spadix length.
Correlation coefficient significant at P ≦ 0.05, 0.01; 15 d.f. when r = 0.482 and 0.606, respectively.

Vase-life did not significantly correlate with any of the other characteristics. It had correlation coefficients of r=−0.45 with abaxial stomata density and r=−0.47 with abaxial hydathode density, but the values were not significant at P≦0.05. Stepwise multiple regression analyses on the dependence of vase-life and time to spadix necrosis on morpho-physiological characteristics showed that the smallest subset that best predicted vase-life and time to spadix necrosis of cultivars were given by the equations: Vase-life (days)=29.1−1.99 (abaxial stomatal density)+18.3 (green/not green)+18.5 (white/not white). R²-value was 0.803. Spadix necrosis (days)=29.45−1.46 (abaxial stomata density)+15.1 (green/not green)+11.1 (white/not white). The R²-value was 0.73.

Vase-Life Experiment 2 (Wet Season)

Transpiration

Five-day transpiration means for cultivars evaluated in vase-life Experiment 2, during the wet season, are shown in Table 3.10. Differences between cultivars were significant at P≦0.001. There was a steady increase in genotypic coefficient of variation with time (40.8-148.3%) but also a steady decrease in index of differentiation up to day 25, an increase followed by another steady decrease up to day 50. ‘Terra’ had the highest transpiration up to day 40. By day 45, ‘Cuba’ had the highest transpiration. On day 5, ‘Success’ and ‘Lydia’ had the lowest transpiration. ‘Success’ had the lowest transpiration up to day 15. ‘Tropical’ had the lowest transpiration by day 20, and by day 25 to 30 with ‘Fantasia’. ‘Acropolis’, ‘Lydia’ and ‘Tropical’ had the lowest transpiration by day 35 and ‘Evergreen’ by day 40. By day 45 and 50, 62.5% of the cultivars showed no transpiration.

TABLE 28
Five-day means for transpiration, abaxial stomatal density, time to
spadix necrosis, spathe necrosis and vase-life of 16 Anthurium
andraeanum (Hort.) cultivars evaluated in vase-life Experiment 2, the wet season
Experiment 2 (5-day) transpiration averages

| | | | | | | | | | | | Abaxial | | | |
|---|---|---|---|---|---|---|---|---|---|---|---|---|---|---|
| | | | | | | | | | | | stomata | | | Vase- |
| | | | | | | | | | | | density | Spadix | Spathe | life |
| Cultivar | 5 | 10 | 15 | 20 | 25 | 30 | 35 | 40 | 45 | 50 | per mm² | necr.^{a} | necr. | (days) |
| Acropolis | 3.67 | 2.07 | 2.00 | 1.53 | 1.40 | 0.87 | 0.40 | 0.44 | 0.00 | 0.00 | 14.6 | 16 | 21.8 | 16.0 |
| Cuba | 5.20 | 3.20 | 2.80 | 2.80 | 2.60 | 2.73 | 2.67 | 2.13 | 2.20 | 2.20 | 2.6 | 41.4 | 48.7 | 41.0 |
| Evergreen | 5.27 | 3.73 | 3.80 | 3.53 | 1.60 | 0.67 | 0.67 | 0.11 | 0.00 | 0.00 | 24.5 | 21.4 | 21.7 | 21.0 |
| Fantasia | 4.67 | 2.53 | 1.27 | 1.40 | 0.67 | 0.40 | 0.67 | 0.67 | 0.00 | 0.00 | 16.2 | 14.2 | 14.4 | 14.2 |
| Fla Range | 4.53 | 2.80 | 2.13 | 1.47 | 1.20 | 1.47 | 1.47 | 0.78 | 0.00 | 0.00 | 2.8 | 22 | 30.7 | 22.0 |
| Honduras | 3.33 | 2.33 | 2.13 | 1.73 | 1.47 | 1.60 | 1.33 | 1.24 | 1.07 | 0.93 | 4.2 | 37.6 | 47.2 | 37.7 |
| Local Pink | 8.60 | 5.27 | 3.93 | 3.07 | 2.33 | 1.93 | 1.47 | 0.89 | 0.00 | 0.00 | 2.5 | 21 | 28.4 | 21.0 |
| Lydia | 2.87 | 1.80 | 1.33 | 1.23 | 1.07 | 0.53 | 0.40 | 0.89 | 0.00 | 0.00 | 4.3 | 15.3 | 16.6 | 15.0 |
| Midori | 4.93 | 3.13 | 3.00 | 2.67 | 2.47 | 2.33 | 2.13 | 1.80 | 1.43 | 1.53 | 6.2 | 48.7 | 49.4 | 48.7 |
| Mirjam | 3.20 | 2.07 | 1.53 | 1.13 | 1.20 | 1.27 | 0.73 | 0.78 | 0.00 | 0.00 | 6.9 | 15 | 24.1 | 15.0 |
| Pierrot | 4.87 | 3.13 | 2.33 | 2.27 | 1.73 | 1.87 | 2.00 | 2.00 | 1.07 | 1.07 | 6.5 | 34.3 | 42.2 | 34.3 |
| Success | 2.87 | 1.73 | 1.13 | 1.40 | 0.93 | 1.20 | 1.33 | 0.44 | 0.00 | 0.00 | 5.6 | 19 | 32.3 | 19.0 |
| Tequila | 5.00 | 3.20 | 3.07 | 2.93 | 2.53 | 2.73 | 2.33 | 1.60 | 1.80 | 1.27 | 11.2 | 21.5 | 28.8 | 21.5 |
| Terra | 10.00 | 6.40 | 5.40 | 4.80 | 3.93 | 3.87 | 3.47 | 2.27 | 0.83 | 0.67 | 6.6 | 30 | 39.0 | 30.0 |
| Tropical | 3.47 | 2.27 | 1.47 | 1.07 | 0.67 | 0.40 | 0.40 | 0.56 | 0.00 | 0.00 | 6.2 | 14.3 | 16.2 | 14.0 |
| Venus | 4.27 | 2.67 | 2.60 | 1.93 | 1.73 | 1.27 | 1.40 | 0.67 | 0.00 | 0.00 | 5.6 | 20.3 | 23.3 | 18.0 |
| ^{b}Sig (P ≦) | 0.001 | 0.001 | 0.001 | 0.001 | 0.001 | 0.001 | 0.001 | 0.001 | 0.001 | 0.001 | 0.001 | 0.001 | 0.001 | 0.001 |
| Mean | 4.80 | 3.02 | 2.50 | 2.19 | 1.72 | 1.57 | 1.43 | 1.08 | 0.53 | 0.48 | 7.4 | 23.1 | 30.3 | 22.8 |
| SEM | 0.434 | 0.288 | 0.310 | 0.366 | 0.325 | 0.257 | 0.233 | 0.268 | 0.304 | 0.264 | 0.954 | 2.339 | 4.45 | 2.26 |
| LSD_{(0.05)} | 1.03 | 0.68 | 0.74 | 0.87 | 0.77 | 0.61 | 0.55 | 0.64 | 0.72 | 0.63 | 2.27 | 5.56 | 10.6 | 5.37 |
| Genotypic | 40.8 | 41.4 | 46.4 | 47.5 | 50.3 | 62.0 | 63.0 | 62.4 | 145.0 | 148.3 | 80.4 | 44.5 | 39.0 | 45.6 |
| CV (%) | | | | | | | | | | | | | | |
| ID^{c} | 2.62 | 2.56 | 2.21 | 1.67 | 1.52 | 2.11 | 2.11 | 1.40 | 1.36 | 1.46 | 4.54 | 2.66 | 1.55 | 2.81 |

^{a}necrosis,
^{b}Significance,
^{c}Index of differentiation.

The differences between cultivars for abaxial stomata density, time to spadix and spathe necrosis, and vase-life were significant (P≦0.001). Abaxial stomata density had the highest genotypic coefficient of variation (80.4%) and also the highest index of differentiation.

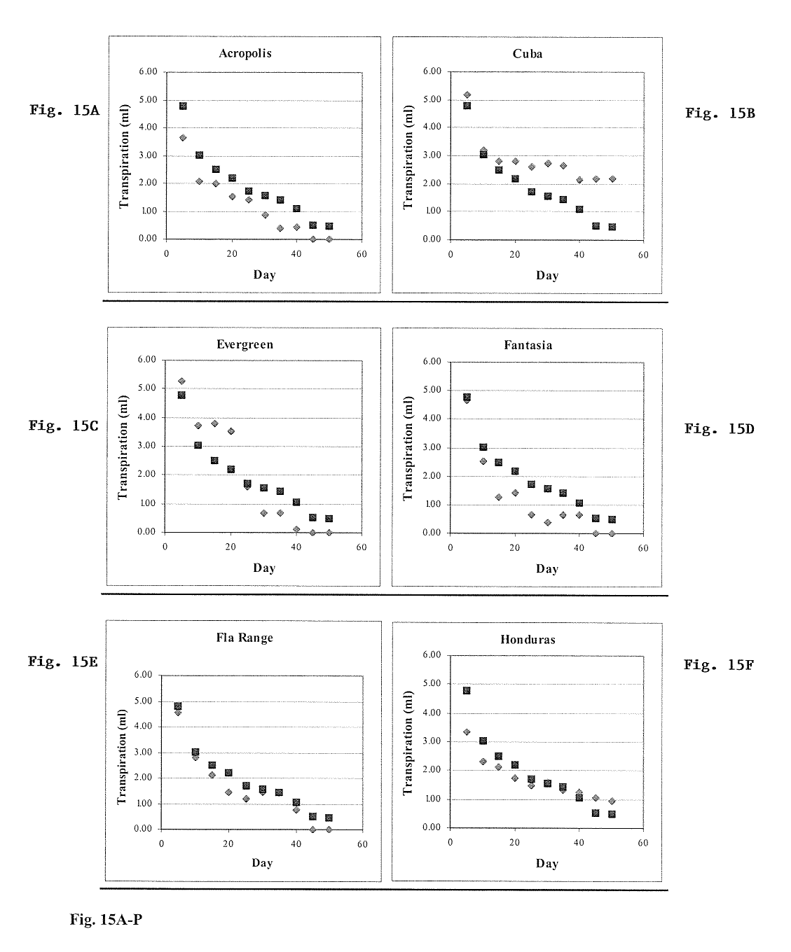

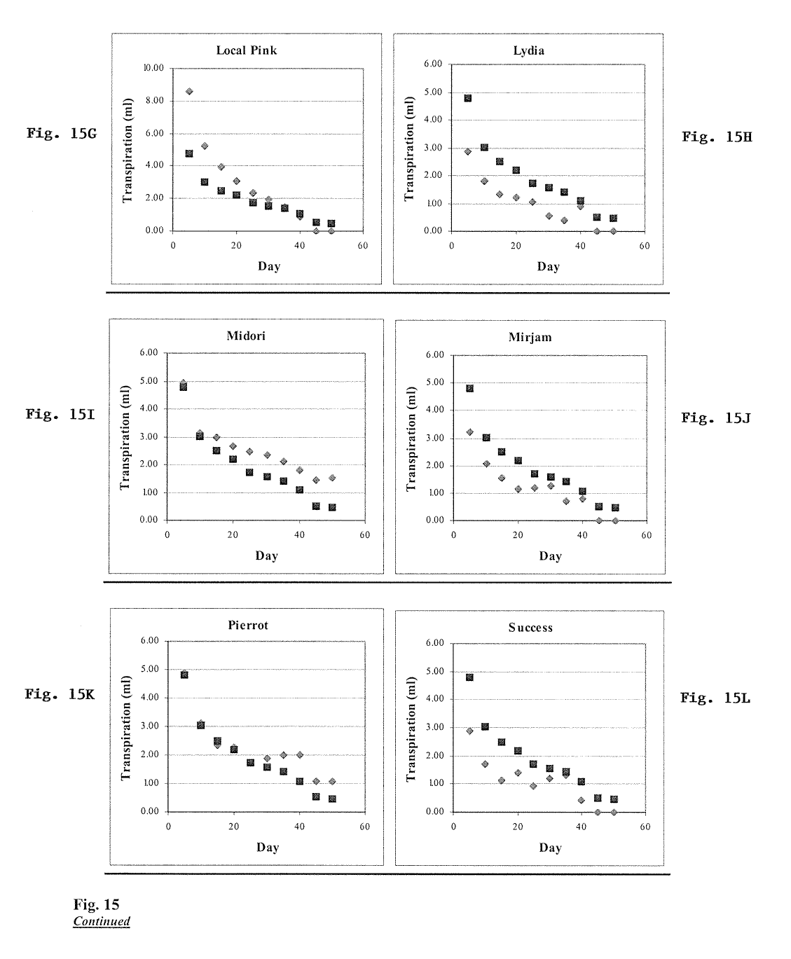

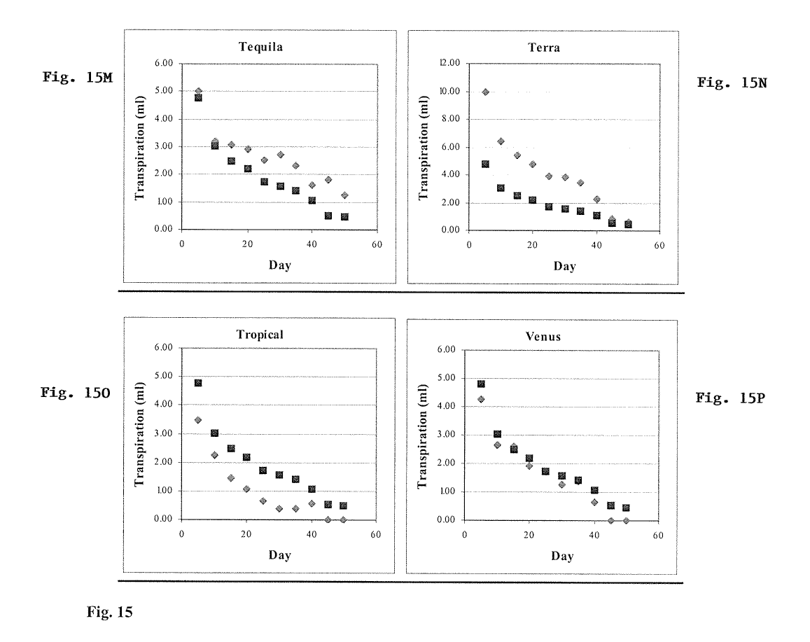

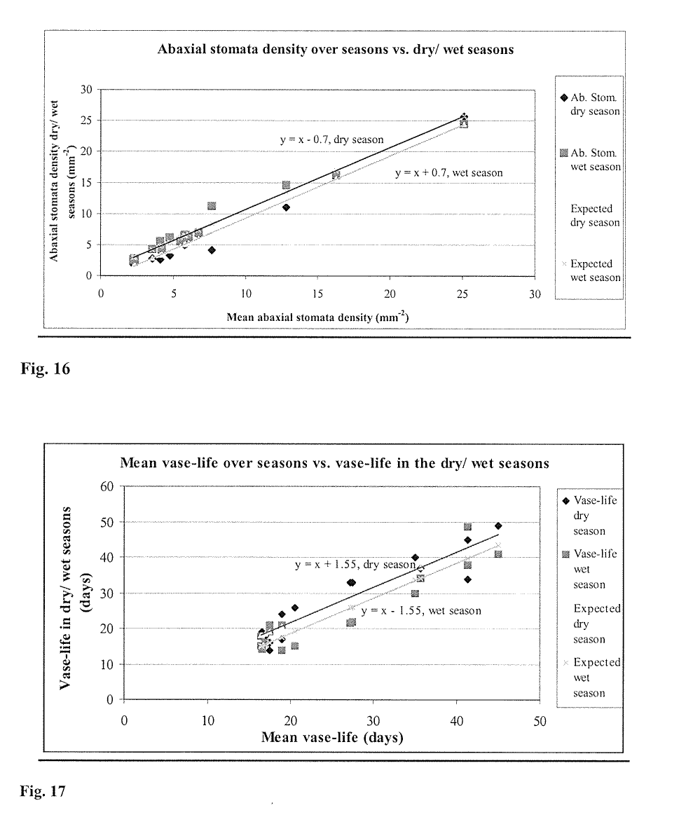

FIG. 15 shows the scatter plots for 5-day transpiration averages of the 16 anthurium cultivars evaluated in the wet season as well as the scatter plot of the mean of all cultivars as a reference. Cultivars with short vase-lives had transpiration values below the general mean of cultivars as in ‘Acropolis’, ‘Evergreen’, ‘Fantasia’, ‘Fla Range’, ‘Lydia’, ‘Mirjam’, ‘Success’, ‘Tropical’ and ‘Venus’. Cultivars with long vase-lives had transpiration values generally greater than the mean transpiration such as in ‘Cuba’, ‘Midori’, ‘Pierrot’, and ‘Terra’. The cultivar, ‘Honduras’ initially had transpiration values lower than the general mean, but by day 40 had values greater than the general mean. ‘Tequila’ had a low vase-life although it had transpiration values greater than the general mean; however, like ‘Local Pink’ it did not have a steady state transpiration period.

The data of Table 29 demonstrate that cultivars with shorter vase-lives took a relatively long time to reach steady state transpiration (>30 days). These cultivars also had shorter duration of steady state transpiration as well as low steady state transpiration values. The opposite scenarios were seen for cultivars with longer vase-lives.

Correlation Between Characteristics

Day 5 and 15 transpiration values (Table 3.12) had significant correlation (P≦0.05) with steady state transpiration (r=0.58, 0.59, respectively), whereas all the other day values, including mean 5 to 50 day transpiration values, had significant correlation (P≦0.01) with steady state transpiration (r=0.60 to 0.87). Days 25 to 50, including mean 5 to 50 day transpiration values, had significant correlation (P≦0.05, 0.01) with time to spadix and spathe necrosis.

DAI had significant correlation (P≦0.05, 0.01) with transpiration values at day 40, 45, 50, and time to spadix necrosis.

TABLE 29
Commencement of steady state transpiration, duration, steady state
transpiration values, abaxial stomata density, time to spadix and
spathe necrosis and vase-life of 16 Anthurium andraeanum (Hort.)
cultivars evaluated in vase-life Experiment 2, the wet season

| | | Duration | | Abaxial stomata | Days to spadix | Days to | Vase-life |
|---|---|---|---|---|---|---|---|
| Cultivar | DAI^{a} | (days)^{b} | SST^{c} (ml) | density per mm² | necr.^{d} | spathe necr.^{d} | (days) |
| Acropolis | 35 | 5 | 0.40 | 14.6 | 16.0 | 21.8 | 16 |
| Cuba | 15 | 20 | 2.80 | 2.6 | 41.4 | 48.7 | 41 |
| Evergreen | 30 | 5 | 0.67 | 24.5 | 21.4 | 21.7 | 21 |
| Fantasia | 35 | 5 | 0.67 | 16.2 | 14.2 | 14.4 | 14 |
| Fla Range | 45 | 5 | 0.00 | 2.8 | 22.0 | 30.7 | 22 |
| Honduras | —^{e} | —^{e} | —^{e} | 4.2 | 37.6 | 47.2 | 38 |
| Local Pink | 45 | 5 | 0.00 | 2.5 | 21.0 | 28.4 | 21 |
| Lydia | 45 | 5 | 0.00 | 4.3 | 15.3 | 16.6 | 15 |
| Midori | —^{e} | —^{e} | —^{e} | 6.2 | 48.7 | 49.4 | 49 |
| Mirjam | 35 | 5 | 0.78 | 6.9 | 15.0 | 24.1 | 15 |
| Pierrot | 35 | 5 | 2.00 | 6.5 | 34.3 | 42.2 | 34 |
| Success | 45 | 5 | 0.00 | 5.6 | 19.0 | 32.3 | 19 |
| Tequila | —^{e} | —^{e} | —^{e} | 11.2 | 21.5 | 28.8 | 22 |
| Terra | 25 | 5 | 3.93 | 6.6 | 30.0 | 39.0 | 30 |
| Tropical | 30 | 5 | 0.40 | 6.2 | 14.3 | 16.2 | 14 |
| Venus | 45 | 5 | 0.00 | 5.6 | 20.3 | 23.3 | 18 |

^{a}Day after initiation of experiment to commencement of steady state transpiration;
^{b}Duration of steady state transpiration;
^{c}Steady state transpiration;
^{d}necrosis.
‘—^{e}’ means that the cultivar had no steady state transpiration, therefore no DAI and no duration.

TABLE 30
Genetic correlation between vase-life and morpho-physiological
characteristics measured in vase-life Experiment 2

| | | | | Spathe | | | | |
|---|---|---|---|---|---|---|---|---|
| Day | Mean 5-50 | Ab. stom.^{b} | Spad. necr.^{c} | necr.^{d} | DAI^{e} | Duration^{f} | SST^{g} | Vase-life |
| ^{a}D 5 | 0.87 | −0.04 | 0.24 | 0.25 | −0.27 | 0.04 | 0.58 | 0.24 |
| D 10 | 0.89 | −0.02 | 0.28 | 0.28 | −0.3 | 0.03 | 0.60 | 0.28 |
| D 15 | 0.91 | 0.09 | 0.38 | 0.36 | −0.35 | 0.09 | 0.59 | 0.38 |
| D 20 | 0.93 | 0.17 | 0.44 | 0.41 | −0.45 | 0.18 | 0.69 | 0.43 |
| D 25 | 0.97 | −0.16 | 0.57 | 0.58 | −0.46 | 0.33 | 0.78 | 0.56 |
| D 30 | 0.92 | −0.35 | 0.63 | 0.72 | −0.44 | 0.39 | 0.82 | 0.63 |
| D 35 | 0.90 | −0.34 | 0.67 | 0.74 | −0.43 | 0.43 | 0.82 | 0.67 |
| D 40 | 0.77 | −0.42 | 0.75 | 0.76 | −0.53 | 0.50 | 0.87 | 0.75 |
| D 45 | 0.61 | −0.22 | 0.78 | 0.77 | −0.74 | 0.85 | 0.79 | 0.79 |
| D 50 | 0.59 | −0.25 | 0.85 | 0.81 | −0.73 | 0.86 | 0.75 | 0.85 |
| | Mean 5-50 | −0.14 | 0.58 | 0.59 | −0.49 | 0.32 | 0.80 | 0.58 |
| | | Ab. stom. | −0.30 | −0.43 | −0.20 | −0.25 | −0.07 | −0.30 |
| | | | Spad. necr. | 0.94 | −0.58 | 0.70 | 0.76 | 1.00 |
| | | | | Spathe | −0.44 | 0.60 | 0.70 | 0.95 |
| | | | | necr. | | | | |
| | | | | | DAI | −0.67 | −0.79 | −0.59 |
| | | | | | | Duration | 0.46 | 0.69 |
| | | | | | | | SST | 0.77 |

^{a}day;
^{b}Abaxial stomata density;
^{c}Spadix necrosis;
^{d}Spathe necrosis;
^{e}Day after initiation of experiment to commencement of steady state transpiration;
^{f}Duration of steady state transpiration;
^{g}Steady state transpiration.
Correlation coefficient significant at P ≦ 0.05 and 0.01 when r = 0.497 and 0.623, respectively.

Duration of steady state transpiration (Table 30) had significant correlation (P≦0.05 to 0.01) with day 40, 45, 50, spadix necrosis, spathe necrosis, and DAI. Steady state transpiration had significant correlation (P≦0.05 to 0.01) with all the attributes measured except abaxial stomata density and duration of steady state transpiration. Vase-life had significant correlation (P≦0.05 to 0.01) with all attributes measured except day 5 to day 20 transpiration values, and abaxial stomata density.

Stability of Abaxial Stomata Density Over Season

The cultivar differences for abaxial stomata density in vase-life Experiment 2 (wet season) were significant (P≦0.001) (Table 28). The cultivar, ‘Evergreen’ again had the largest number of abaxial stomata and ‘Local Pink’ had the least. The cultivar, ‘Tropical’ and ‘Tequila’ had considerably more in the wet season experiment than in the dry (vase-life Experiment 1). Nevertheless, the correlation of stomata density in the wet versus that in the dry season was high (Pearson's, r=0.95; Spearman's Rank, r=0.76).

Furthermore, an ANOVA showed that genotype×season interactions were not significant (P≦0.05) for stomata density. Cultivar differences were nevertheless significant (P≦0.001). A comparison of regression lines (Table 3.13) showed that the two regression lines for abaxial stomata density (FIG. 16 [3.6]) over both seasons could be explained with two parallel regression lines (y=x−0.70, dry season; y=x+0.70, wet season) suggesting that there were no cultivar-season interactions. The equations showed that abaxial stomata density increased by one stoma mm⁻² in the wet season suggesting environmental influence on abaxial stomata density. Cultivar differences for abaxial stomata density were nevertheless significant (P<0.001) in the wet season.

On an average the stomata density in the wet season (7.9 mm⁻²) was higher than that in the dry season (6.7 mm⁻²), but was not significant (P<0.05). This suggests there was a close correspondence between stomata densities in both seasons.

TABLE 31
Comparison of regression lines for spathe abaxial stomata density for
16 Anthurium andraeanum (Hort.) cultivars evaluated for vase-life in
the dry and wet seasons.
Abaxial stomata density

| Source | DF | SS | MS | VR |
|---|---|---|---|---|
| Residual (pooled) | 28 | 28.1984 | 1.0071 | |
| Between slopes | 1 | 1.2079 | 1.2079 | 1.199 |
| Residual (//lines) | 29 | 29.4064 | 1.014 | |
| Between adj. means | 1 | 15.8203 | 15.8203 | 15.602 |
| Residual (overall) | 30 | 45.2267 | 1.5076 | |
| Overall regression | 1 | 1105.995 | 1105.995 | |
| Total | 31 | 1151.221 | | |

Ab. Stom. = Abaxial stomata density.

Stability of Vase-Life, Time to Spadix and Spathe Necrosis Over Seasons

Vase-life in the wet season ranged from 14 days in the cultivars, ‘Fantasia’ and ‘Tropical’ to 49 days in ‘Midori’ with a mean of 24.3 days (Table 29). The standard error values obtained for vase-life in the wet (2.26) and dry (2.29) seasons were essentially the same. Most cultivars (75%) showed a decrease in vase-life in the wet season. The vase-life of ‘Lydia’ and ‘Tropical’ were reduced by almost half the dry season value in the wet season. However, the vase-life of ‘Evergreen’ increased by 7 days in the wet season. There was a general correspondence between vase-life of cultivars over the two seasons (Pearson's r=0.76; Spearman's r=0.65). An ANOVA on vase-life data showed that both cultivar and cultivar×season differences for vase-life were significant (P≦0.001). However, comparison of regression lines, Table 32, showed that the two regression lines for vase-life over the dry and wet seasons in FIG. 17 could be explained by two parallel lines (y=x+1.55, dry season; y=x−1.55; wet season), suggesting that there were no cultivar-season interactions for vase-life. There was environmental influence on vase-life, with vase-life in the dry season being an average of 3 days more than in the wet season.

TABLE 32
Comparison of regression lines for vase-life for 16 Anthurium
andraeanum (Hort.) cultivars evaluated in the dry and wet seasons

| Source | DF | SS | MS | VR |
|---|---|---|---|---|
| Residual (pooled) | 28 | 436.1609 | 15.5772 | |
| Between slopes | 1 | 3.9392 | 3.9392 | 0.253 |
| Residual (//lines) | 29 | 440.1001 | 15.1759 | |
| Between adj. means | 1 | 76.8799 | 76.8799 | 5.066 |
| Residual (overall) | 30 | 516.98 | 17.2327 | |
| Overall regression | 1 | 3264.6 | 3264.6 | |
| Total | 31 | 3781.58 | | |

Time to spadix necrosis in the dry season had significant correlation (P≦0.01) with that in the wet season (r=0.78) indicating that this parameter of vase-life is stable over seasons. An ANOVA for time to spadix necrosis over seasons suggest significant genotype×season interactions at P≦0.001. However, comparison of regression lines (Table 33, FIG. 18) show that spadix necrosis during each season can be explained by two parallel regression lines (y=x+1.81, dry season; y=x−1.81, wet season), suggesting no cultivar-season interactions.

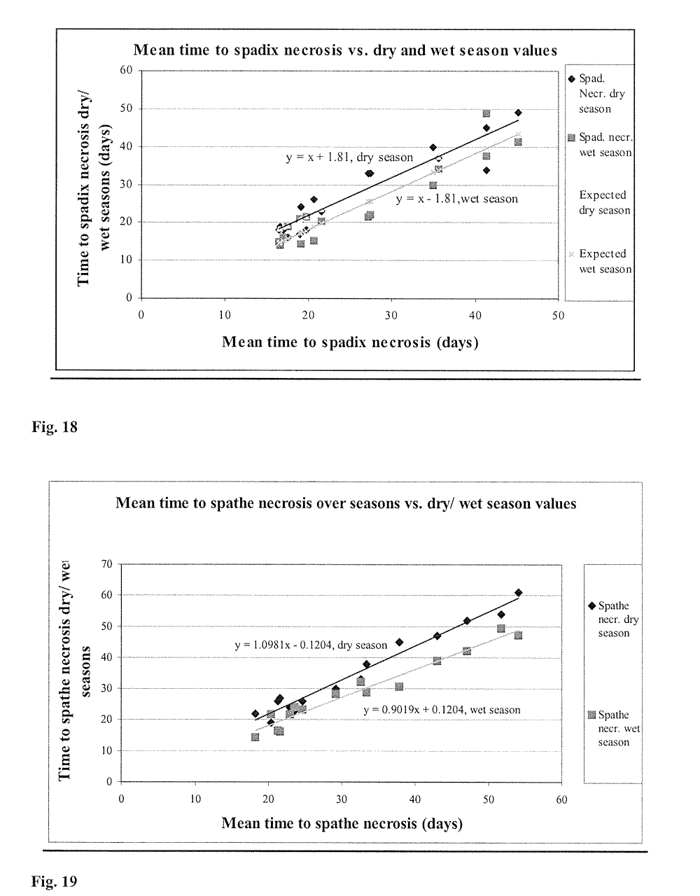

Time to spathe necrosis in the dry season had significant correlation (P≦0.01) with time to spathe necrosis in the wet season (r=0.92), suggesting that this character increases or decreases in a linear manner over seasons. Comparison of regression lines (Table 34, FIG. 19), shows that the two individual regression lines (y=1.098x−0.12, dry season; y=0.902x+0.12, wet season) cannot be written as two parallel lines or one regression line, suggesting cultivar-environment interactions at the 5% level. This was also confirmed by ANOVA, which showed significant genotype×season interactions at P≦0.001.

TABLE 33
Comparison of regression lines for spadix necrosis for 16 Anthurium
andraeanum (Hort.) cultivars evaluated in the dry and wet seasons

| Source | DF | SS | MS | VR |
|---|---|---|---|---|
| Residual (pooled) | 28 | 383.6512 | 13.7018 | |
| Between slopes | 1 | 0.0336 | 0.0336 | 0.002 |
| Residual (//lines) | 29 | 383.6848 | 13.2305 | |
| Between adj. means | 1 | 105.125 | 105.125 | 7.946 |
| Residual (overall) | 30 | 488.8098 | 16.2937 | |
| Overall regression | 1 | 3059.485 | | |
| Total | 31 | 3548.295 | | |

TABLE 34
Comparison of regression lines for spathe necrosis for 15 Anthurium
andraeanum (Hort.) cultivars evaluated in the dry and wet seasons

| Source | DF | SS | MS | VR |
|---|---|---|---|---|
| Residual (pooled) | 26 | 159.1399 | 6.1208 | |
| Between slopes | 1 | 39.0376 | 39.0376 | 6.378 |
| Residual (//lines) | 27 | 198.1775 | 7.3399 | |
| Between adj. means | 1 | 275.4272 | 275.4272 | 37.525 |
| Residual (overall) | 28 | 473.6047 | 16.9145 | |
| Overall regression | 1 | 4053.685 | 4053.685 | |
| Total | 29 | 3548.295 | | |

Correlation Between Abaxial Stomata Number, Vase-Life and Spadix Necrosis

There was significant positive correlation (P≦0.01) between abaxial stomata densities for cultivars evaluated over both seasons (r=0.94). There was also significant positive correlation (P≦0.01) between vase-life of cultivars evaluated over the wet and dry seasons (r=0.78). Correlation between vase-life and abaxial stomatal density for cultivars evaluated over the wet season was low and not significant (r=−0.30). Time to spadix necrosis had significant correlation with vase-life (P≦0.001) with r=1.00.

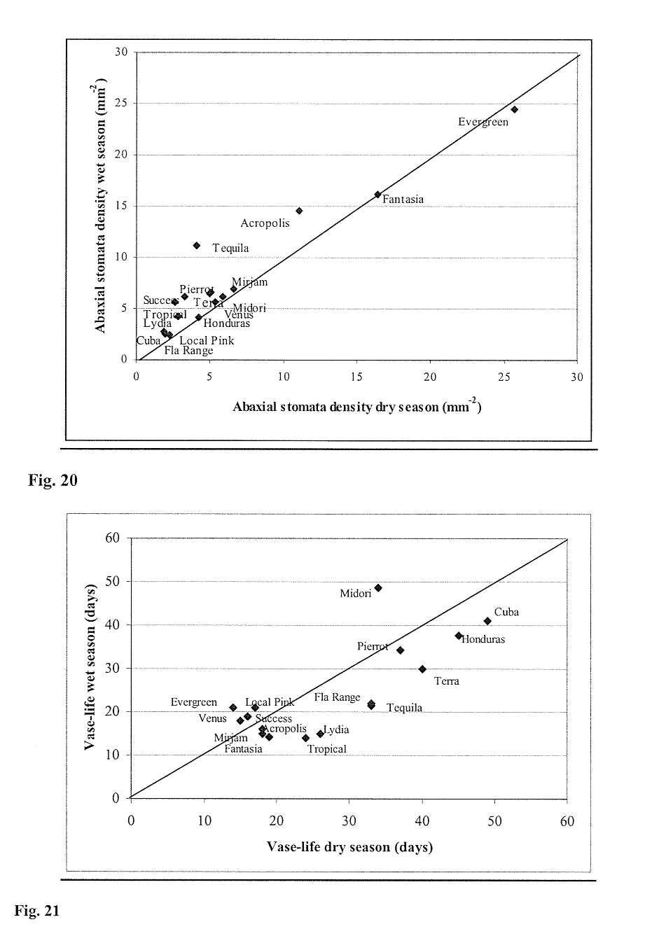

The data of FIG. 20 demonstrate that the majority of cultivars had abaxial stomata density of below 10/mm² over both seasons. Only the cultivars, ‘Tequila’ and ‘Acropolis’ had substantially more abaxial stomata per mm² in the wet season. There was much wider variation for vase-life (FIG. 21 [3.11]) with only 5 cultivars (‘Midori’, ‘Local Pink’, ‘Evergreen’, ‘Venus’ and ‘Success’) having better vase-life in the wet season.

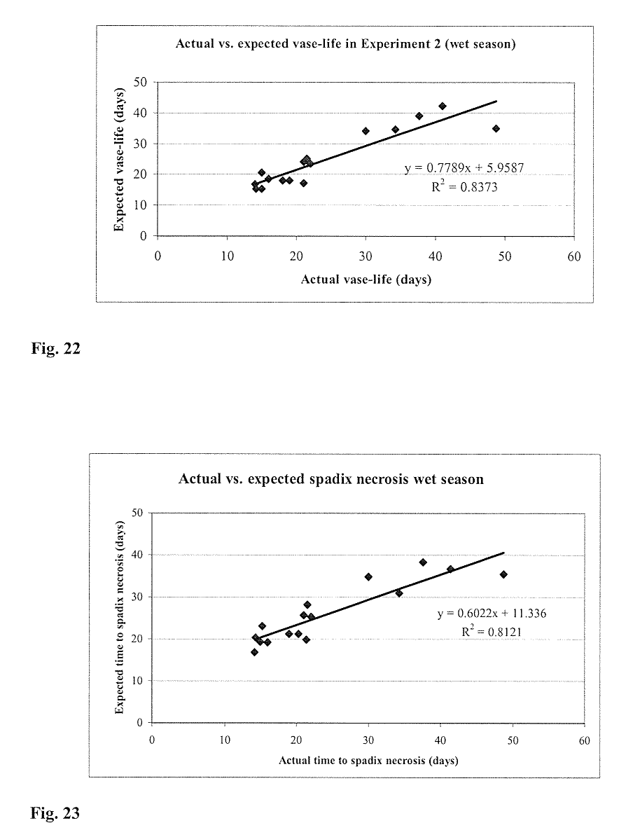

The data of FIG. 22 demonstrate that the prediction equation developed from the first vase-life experiment in the dry season was able to predict vase-life of cultivars in the second vase-life experiment conducted in the wet season (R^{2=0.84}). The results show that variation in spathe abaxial stomatal density between seasons explained most of the variation in vase-life observed over the two seasons.

The data of FIG. 23 demonstrate that the regression equation developed for time to spadix necrosis from the first vase-life experiment (dry season) was also able to predict vase-life of cultivars in the wet season (R^{2=0.81}).

Experiment 3—Prediction Experiment

The prediction equation developed in the previous experiment was tested on nine other anthurium cultivars (Table 19). The cultivar differences for both abaxial stomata density and vase-life were significant (P≦0.01) (Table 35). The red-obake cultivar, ‘Kalapana’, had the most abaxial stomata whereas the cultivar, ‘Lunette’, had the least. Though not shown, the distribution of abaxial stomata in the red and green regions of the ‘Kalapana’ spathes was not uniform. There were many more in the red portion compared to the green. The cultivars, ‘Champagne’ and ‘Sweety’ had the longest vase-lives (21 days) whereas ‘Ibara’ and ‘Kalapana’ had the shortest (10 days).

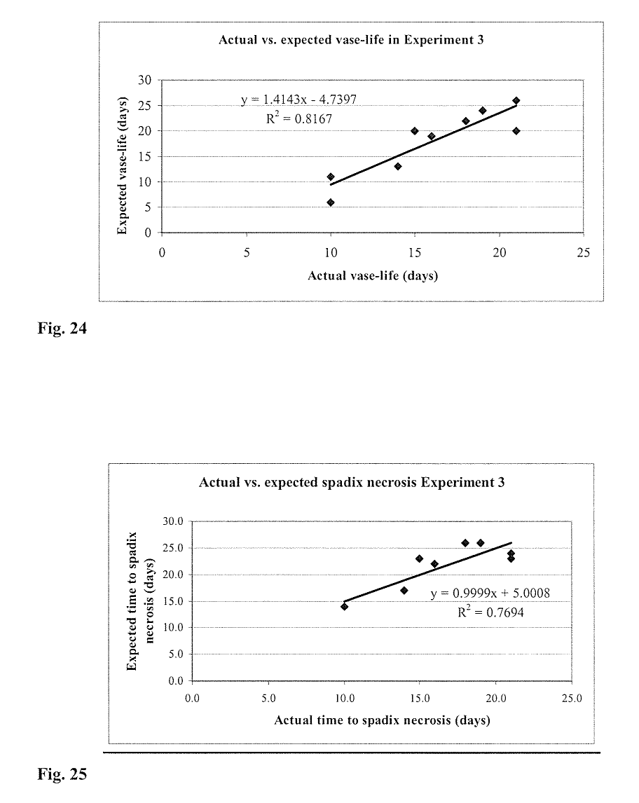

The data of FIG. 24 demonstrate that 82% of the variation in vase-life of the nine cultivars (Experiment 3) can be explained by the vase-life prediction equation. This result shows that the vase-life prediction equation can predict vase-life in a new set of anthurium cultivars, indicating that the equation is valid.

The data of FIG. 25 demonstrate that the regression equation developed for spadix necrosis in vase-life Experiment 1 in the dry season was able to explain time to spadix necrosis in a new set of nine other cultivars in the late wet season (R²=0.77). This showed that the spadix necrosis prediction equation developed for time to spadix necrosis is valid.

TABLE 35
Abaxial stomata density, time to spadix necrosis and actual vase-life
of 9 Anthurium andraeanum (Hort.) cultivars used in Experiment 3,
the prediction experiment

| | Abaxial | | |
|---|---|---|---|
| | stomata density | Time to spadix | Vase-life |
| Cultivar | (mm⁻²) | necrosis (days) | (days) |
| Champagne | 11.1 | 21.0 | 21 |
| Cheers | 4.7 | 15.0 | 15 |
| Gloria | 5.0 | 16.3 | 16 |
| Ibara | 18.6 | 10.0 | 10 |
| Kalapana | 20.9 | 10.0 | 10 |
| Lunette | 2.6 | 19.0 | 19 |
| Rosa | 8.3 | 14.0 | 14 |
| Senator | 12.7 | 18.0 | 18 |
| Sweety | 4.7 | 21.0 | 21 |
| Significance (P≦) | 0.001 | 0.001 | 0.001 |
| Mean | 9.83 | 16.05 | 16 |
| SEM | 0.76 | 0.86 | 0.86 |
| LSD_{(0.05)} | 2.39 | 2.09 | 2.09 |
| Genotypic CV (%) | 66.2 | 26.6 | 26.7 |
| ID^{a} | 4.88 | 2.86 | 2.84 |

^{a}= Index of differentiation.

Criteria for Assessment of Vase-Life

The post-harvest life of anthurium cut-flowers is usually determined by the onset of one of a number of senescence symptoms: loss of spathe glossiness, blueing of spathe, spadix dehydration and necrosis, spathe wilting, browning of the peduncle base and stem collapse (Shirakawa et al, 1964, Proc. Am. Soc. Hort. Sci., 85:642-646; Paull, 1982, HortSci. 17(4):606-607; Paull et al, 1985, J. Am. Soc. Hort. Sci., 110:156-162). Furthermore, the point of termination of vase-life can be preset at the first sign of senescence symptoms (Mayak and Dilley, 1976, J. Am. Soc. Hort. Sci. 101:583-585) or fixed up to the point of 50% loss of cut-flower quality (Paull and Goo, 1985, J. Am. Soc. Hort. Sci., 110:84-88), or extended until the total death of cut-flowers (Salinger, 1975, Acta Horticulturae 41:207-216). This makes the measure of vase-life complex, less precise and therefore not amenable for use in breeding programs.

This study for the first time investigated senescence symptoms in a wide range of anthurium cultivars. Although the cultivars showed a variety of symptoms viz. peduncle base browning, loss of glossiness, spathe wilting (floppiness), spadix and spathe necrosis and spathe discoloration; in conformity with previous studies (Paull et al., 1985, supra), peduncle base browning, spadix necrosis and spathe necrosis were the only signs of senescence that were common to all cultivars.

Peduncle base browning was the first senescence symptom (8-15 DAI) to appear in anthurium cultivars, but it neither discriminated between the cultivars nor correlated with time to onset of spathe or spadix symptoms, and therefore was not considered a good measure of vase-life. Spadix necrosis was usually the second symptom (14-49 DAI) to appear in anthurium cut-flowers. This, apart from being discriminatory, correlated very well with the onset of spathe necrosis and spathe discoloration, which were either delayed in a cultivar specific manner or did not occur at all. Importantly, the vase-life of 82% of the cultivars was terminated because of spadix necrosis.

The study demonstrates that time to spadix necrosis is the best measure of vase-life in anthurium cut-flowers for several reasons. It is the earliest occurring senescence symptom that is common to all anthurium cultivars, has the best differentiation index and is highly correlated to all other important senescence symptoms. Further, the study demonstrates that vase-life determined by a more complex criterion correlated well with vase-life determined by spadix necrosis, and has a similar error estimate. It is recommended that vase-life should therefore be based on the first sign of spadix necrosis in two out of the three cut-flowers contained in each experimental unit.

Water Status and Vase-Life

Water relations in cut-flowers is considered to be one of the main factors affecting vase-life, and is a function of the water content at harvest, and the rates of water uptake and water loss after harvest (Halevy and Mayak, 1979, Hort. Rev., 1:204-236). The clear association of daily transpiration rates during the 25-45 day period with vase-life and the strong correlation between dehydration-induced necrosis of the spadix with other senescence symptoms in Experiments 1 and 2 supports earlier findings that senescence symptoms in anthurium are attributed to water stress.

The transpiration rate was high initially but steadily decreased in all cultivars. The present study supports previous findings by Paull and Goo (Paull and Goo, 1985, J. Am. Soc. Hort. Sci., 110:84-88), that vascular occlusion may not be due to bacteria or air clogging. Firstly, the precautions taken during the experimentation process preclude the influence of these factors and secondly, the relatively low error (low CV_{within}) for vase-life observed in this study does not point to factors other than experimental factors influencing vase-life.

The rate of water loss due to transpiration during the first 15 days correlated with abaxial stomata density and showed strong positive trends with spathe size (Table 24, but the relationship with stomatal density and spathe size became progressively weaker with subsequent assessments of transpiration. Hence, at the end of the 15 day period, the water status of anthurium cut-flowers would have been in favor of cultivars with less abaxial stomata numbers.

The progressively stronger correlation between vase-life and mean daily transpiration rates, during the period 25-45 DAI, showed that cultivars that maintained a high transpiration rate over a longer time had delayed symptoms of water stress. This was evident in the transpiration rate curve as an inflection, resulting in the leveling off of transpiration rates. The study showed that cultivars which maintained a high enough steady state transpiration rate for long periods of time had delayed symptoms of desiccation-induced senescence in Experiments 1 and 2.

The results indicate that the vascular occlusion process may be stalled for some time, depending on the anthurium cultivar. This allows some anthurium cultivars to maintain water status by allowing water intake and transpiration, therefore extending their vase-life. The duration of steady state transpiration was correlated to the color of the spathe, with the non-reds, and particularly the greens, maintaining steady state transpiration for longer periods.

The two processes that reflect the water status of the anthurium cut-flower, viz. (i) transpiration loss during the initial period of 0-15 days, which is influenced by stomata density and (ii) the timing and duration of steady state transpiration during the period 20-45 days, which is affected by cut-flower color; were represented in the prediction equation for vase-life through abaxial stomata density and cut-flower color. This indicates an integral role for these processes. Furthermore, the steady state transpiration levels achieved were also negatively correlated to abaxial stomata density (Table 26). Hence, cultivars with relatively short vase-lives either had an above average transpiration rate during the early period and a relatively low steady state transpiration and/or a very short steady state duration of transpiration (FIG. 11).

A Role for Spathe Color

The inclusion of green/not green in the prediction equation for vase-life in this study indicates a direct or indirect role for carbohydrates in the senescence process. The green-spathed cultivars performed better in vase-life experiments than the reds, oranges and pinks. The red cultivar ‘Honduras’, which has a green co-pigmentation also performed well in vase-life experiments. As the senescence symptoms in anthurium cut-flowers are largely due to water stress, it may well be that carbohydrate status may be involved in the regulation of stomatal function.

Similarly, the variable, spathe white/non-white, was also important in the prediction of anthurium vase-life. The positive relationship of white-spathed cultivars with the duration of steady state transpiration compared to a significant (P<0.05) negative relationship obtained for red-spathed cultivars indicated that the whites were able to exert greater regulatory control over the rapidly declining transpiration rate than the red group. The red group showed typical blueing of spathe during senescence in this study.

Water Stress a Role for Stomatal Regulation

Abaxial stomata density was correlated with transpiration rate during the early stages (1-15 DAI) after cutting, but was not correlated during the latter stages. Abaxial stomata density was however, negatively correlated to steady state transpiration rates achieved in anthurium cultivars, and accounted for about 21% of the total variation in vase-life in vase-life Experiment 1. These observations indicate that stomata may have a role in the latter stages following partial vascular occlusion. The relationship of spathe color with duration of steady state transpiration and its implication to carbohydrate status and cell membrane integrity indicate a role for stomata regulation in vase-life.

Transpiration during the latter stages was positively correlated to spadix length and negatively correlated to peduncle length. In anthurium, the spadix is the major site of water loss, accounting for as much as 50-60% of the total water loss; the spathe accounted for 40% and the peduncle, 10-22% (Paull and Goo, 1985, J. Am. Soc. Hort. Sci., 110:84-88).

Seasonal Stability of Vase-Life

Abaxial stomata-season interactions of cultivars over the wet and dry seasons were found to be not significantly different (P≦0.05) over seasons by ANOVA and comparison of regression lines. Sixty-nine percent of the 16 cultivars evaluated in Experiments 1 and 2 had more stomata in the wet season. Fifty percent of all cultivars had shorter vase-lives because of the higher abaxial stomatal density. These results may also be translated to drier and wetter parts of the beds.

Cultivar-season interaction for vase-life over seasons was significant at P≦0.05 using ANOVA, but a comparison of regression lines of mean performance over seasons showed that there was none. Abaxial stomata explained 84% of the variation (based on the vase-life regression equation) over the two seasons (cultivar spathe color remained constant over the two seasons). Some of the other morpho-physiological characters may have changed significantly, for example, hydathode numbers, adaxial stomatal numbers and epicuticular wax content, thus causing some cultivars to change ranks with regards to vase-life.

Since abaxial stomata density is the only characteristic that needs measurement when predicting the vase-life and time to spadix necrosis of anthurium cultivars using the prediction equations, then any variation in vase-life will be detected by an increase or decrease in abaxial stomata density.

Accordingly, the vase-life of a cultivar can be predicted by assaying abaxial stomata density.

Predicting Vase-Life Based on Morpho-Physiological Variables

The data provided herein propose a novel regression equation to predict vase-life based on abaxial stomata density and spathe color/s in anthurium cultivars. The prediction equation performed well over seasons when the same cultivars were used, or when a new set of cultivars was used, indicated by the high coefficient of determination as well as high rank correlation. The robustness of the equation suggests that the prediction equation for vase-life can be used in a wide range of situations for cultivars grown under standard management conditions. The equation is generally applied with the proviso that cut-flowers be obtained from cultivars grown under similar conditions with the same harvesting standard to obtain a similar ranking order as that obtained under a different environment.

Traditionally vase-life evaluation required controlled experiments with replications (Kamemoto and Kuehnle, 1996, Breeding Anthuriums in Hawaii, Honolulu, Hi., U. Hawaii Press), which can last for up to three months. The prediction equation eliminates the need for such vase-life experiments. The prediction equation has terms that are relatively easy to assess. Spathes of cut-flowers of each cultivar can be cleared and the abaxial stomata density counted using a microscope, relatively easily. The spathe color can be readily determined visually. If the stomata density can be assessed without the clearing step, it can further accelerate selection as well as reduce the cost of breeding programs.

The equation indicates that vase-life can vary with the color of the cut-flower being bred. In the equation, colors were broadly classified into groups according to Kamemoto et al. (Kamemoto et al, 1988, U. Hawaii: Research Series 056, HITAHR, Coll. Tropical Agric. And Human Res. 8-88) and Kamemoto and Kuehnle (1996, supra). This classification does not discriminate reds from pinks and oranges from corals. Table 36 shows the variation in vase-life for various color groupings. The red group had the largest number of cultivars (9), followed the white group (6). The results indicate that a minimum acceptable vase-life of three weeks can be achieved in all the colors, despite the fact that the means vary greatly with spathe color. Cultivars with vase-life consistently over three weeks, along with their spathe color are shown in Table 37.

The results provided herein, using a large number of anthurium cultivars grown under standard conditions, demonstrates that spadix necrosis is the most consistent criterion for vase-life assessment and was the one that was best able to discriminate between anthurium cultivars in vase-life experiments. However, results suggest that vascular occlusion in anthurium may not be complete and the ability to maintain steady state transpiration at some level for a longer time seemed to be important in delaying the appearance of water-stress symptoms. Correlation and regression analysis provide circumstantial evidence that this ability may relate to the physiology of stomata regulation.

TABLE 36
Anthurium cultivar groupings for vase-life based on spathe color;
number of cultivars in each color group; mean, range and standard
deviation in each group

| Color group | Number of cultivars | Mean | Range | SD |
|---|---|---|---|---|
| Green and white | 1 | 17.5 | — | — |
| Red and green | 2 | 25.7 | 31.4 | 22.2 |
| Orange and green | 2 | 26.5 | 17.0 | 12.0 |
| Orange | 3 | 19.7 | 12.5 | 6.8 |
| Red | 9 | 18.1 | 7.0 | 2.3 |
| White | 6 | 24.2 | 35.0 | 13.3 |
| Green | 2 | 34.3 | 14.1 | 10.0 |

TABLE 37
Anthurium andraeanum (Hort.) cultivars that showed vase-life
performance over 21 days

| Cultivar | Spathe color | Mean vase-life (days) |
|---|---|---|
| Champagne | White | 25 |
| Fla Range | Orange | 28 |
| Tequila | Green | 30 |
| Pierrot | White | 35 |
| Terra | Bronze | 35 |
| Midori | Green | 41 |
| Honduras | Red and green | 44 |
| Cuba | White | 45 |

Genotype×season interactions for vase-life based on a complex set of criteria, or on time to spadix necrosis alone were found to be significant, suggesting that vase-life and time to spadix necrosis are not stable over seasons, but the mean performance of cultivars over seasons is stable, based on comparison of linear regression lines.

Importantly, these data demonstrate that abaxial stomata number and spathe color can be used to predict the vase-life performance in anthurium, which has the potential for reducing the cost of and simplifying the selection procedures used in anthurium breeding programs.

### Example 3

### Inheritance of the Major Spathe Colors and Productivity in Anthurium

Anthurium is well known for its attractive and long-lasting spathes of various colors, shapes and sizes, which have contributed to its emergence as an important floricultural crop in the tropics. Studies describing the inheritance of spathe and spadix color in Anthurium andraeanum Linden ex André (Kamemoto et al., 1988; Kamemoto and Kuehnle, 1996), may not apply to modern anthurium cultivars, which are, for example, interspecific hybrids between A. andraeanum Linden ex André and other species belonging to the section Calomystrium. Furthermore, there is evidence from molecular biological studies of the flavonoid pathway of anthurium that at least some of the white-spathed cultivars are regulatory mutants. In light of this information, the prevailing genetic model for flower color in anthuriums was evaluated and new models generated for anthurium flower color. The new models are useful for breeding programs for developing and/or propagating anthurium color variants.

The five major spathe colors of anthurium are red, pink, orange, coral (peach) and white. In some cases anthurium spathes can be brown (orange and green), purple, green, have patterns of pink and white, or obake (bi-colored spathes with green lobes and one of the other major spathe colors). Blue and yellow spathe colors are not found in anthurium or its allies.

Carotenoids are found only in the anthurium spadix where they impart bright yellow colors, but all other spadix colors are flavonoid based (Collette, 2002, Ph.D. Thesis, Massey University, Palmerston North, New Zealand). Chlorophylls produce green color in spathes, either alone or in conjunction with orange resulting in brown color shades depending on the concentration of pigments (Kamemoto and Kuehnle, 1996, Breeding Anthuriums in Hawaii, Honolulu, Hawaii: University of Hawaii Press). Hence, the intensity of the green color depends on the presence of chlorophylls without anthocyanins.

Genetics of Spathe Color—‘Kamemoto's Two-Gene Model’

Kamemoto et al. (1988, University of Hawaii: Research Series 056, HITAHR, College of Tropical Agriculture and Human Resources, 8-88), and Kamemoto and Kuehnle (1996, supra) produced evidence for a two-gene model controlling the major spathe colors of anthurium. The genetic model proposed was based on segregation analysis of spathe color of progeny plants derived from crosses of various cultivars of A. andraeanum Linden ex André. Spathe colors were classified as red to pink, orange to coral, and white because of the difficulty at times to separate reds from pinks, and oranges from corals through pigment analysis (Iwata et al., 1979, J. Am. Soc. Hort. Sci. 104:464-466). The two structural genes theorized to control the production of the two main anthocyanin pigments were denoted ‘M’ and ‘O’ with ‘M’ coding for cyanidin 3-rutinoside and ‘O’, the production of pelargonidin 3-rutinoside. They further suggested that recessive epistasis of the ‘O’ locus over the ‘M’ reflects the biochemical pathway for anthocyanin biosynthesis.

### Material and Methods

Hybridization and Experimentation

The location, method of hybridization, nursery practices and after care were similar to those described herein and as practiced by those in the art. Seventy-eight crosses involving 60 parental lines (genotypes) were made and the resulting 3,444 progenies were assessed for cut-flower color over several harvests. For purposes of simplicity all white-spathed and green-spathed genotypes that possessed no anthocyanin were recorded as white, whereas the others were grouped into the major spathe color groups, red and orange, according to Kamemoto et al. (1988, supra) and Kamemoto and Kuehnle (1996, supra), and used in genetic analysis.

Similarly, 15 crosses involving 16 genotypes were used to study the heritability of productivity. Approximately 20 progeny plants per cross were evaluated for productivity over 6 months in a completely randomized design, along with the parental genotypes. The productivity records were extrapolated for a year. Parental genotypes used in the crosses included cultivars with low, moderate and high productivity.

Genetic Analysis

Segregation analysis was conducted for spathe color for each cross against the expected ratios based on Kamemoto's two-gene model, and two other newly proposed models involving a regulatory gene. The expected ratios were compared to observed ratios, based on the above models, using the chi-square test (with Yates' correction) to determine the best model that was able to explain the segregation ratios for spathe color in various crosses.

The proposed new models are different from the model of Kamemoto et al. (1988, supra) in that they invoke a regulatory gene in addition to the structural genes ‘M’ and ‘O’, proposed by Kamemoto et al. In the first model proposed herein, a dominant regulatory gene, ‘R’, which is a repressor that regulates the ‘O’ gene. The ‘R’ gene in the dominant form (R-) therefore represses the ‘O’ gene regardless of its genotypic form (dominant and recessive epistasis) to produce white-spathed flowers. Hence, O-R-, ooR- and oorr should be white and O-rr colored. In the second model proposed here, a dominant regulatory gene ‘R’ that produces a transcriptional activator is assumed, which activates the ‘O’ gene (duplicate recessive epistasis). In this model, genotypes ooR-, oorr and O-rr should be white and O-R-colored. The specific segregation of the colors into red and orange groups in the two newly proposed models follows that of Kamemoto et al. (1988, supra), where ‘MM’ and ‘Mm’ will result in colors belonging to the red group, whereas ‘mm’ will lead to colors belonging to the orange group.

Because the models differ only in the segregation of colored vs. white spathe, the models were first tested for segregation of color:white ratio, and the best fit model was then tested for the segregation of colors, viz. red:orange. Table 38 shows the expected ratios for all possible crosses based on the (a) Kamemoto model, (b) dominant and recessive epistatic model and (c) duplicate recessive epistatic model; Table 39 summarizes the expected ratios for white×white, colored×white and colored×colored crosses under the three models.

Productivity data are presented as the range of genotype productivity together with mid-parent productivity per cross. The coefficient of variation for between cultivar productivity was calculated as the standard deviation between the means for the cultivars divided by the general mean. A histogram was drawn to show the frequency distribution of various productivity levels and another was drawn to compare mean productivity per cross. A mid-parent offspring regression was carried out and an estimate of narrow sense heritability was obtained based on the slope of the fitted regression line. The data analysis was done using the NCSS statistical package.

### Results

Testing Kamemoto's Genetic Model Against Alternative Models for Spathe Color

Tables 38 and Table 39 summarize the most plausible ratios (theoretical analyses) fitted for the segregation (colored vs. white) of spathe colors in white×white, white×colored and colored×colored crosses (Table 41 to Table 49) based on the three genetic models described above. Table 40 shows the most plausible observed ratios for color:white spathed progeny colors based on the three models and the proportion of crosses producing these ratios.

With respect to the white×white cultivar crosses, none of the fitted ratios (Table 41) gave the expected 0:1 (color:white) ratio based on Kamemoto's model (Table 38a), whereas five of the six crosses fitted the duplicate recessive epistasis model (1:3; 1:1) and all six fitted the dominant and recessive epistasis model (1:3; 1:1 and 1:7) (Table 39). Significantly, the new models allow colored-spathed progenies to be produced, in ratios (C:W) of 1:1, 1:3 or 1:7, from crosses between white-spathed cultivars, which is not possible under Kamemoto's model (Table 38a). Although the 0 colored:1 white is the most probable ratio for progenies from white×white crosses (Table 39), based on all three genetic models, none of the six crosses gave this ratio. The possible reasons for this discrepancy are discussed later. The ratios 1:3, 1:1 and 1:7 were equally frequent among the white×white crosses (Table 40).

TABLE 38
The expected segregation ratios for colored:white spathes based on (a)
Kamemoto et al., (b) dominant-and-recessive epistasis and (c) duplicate-
recessive epistasis genetic models.

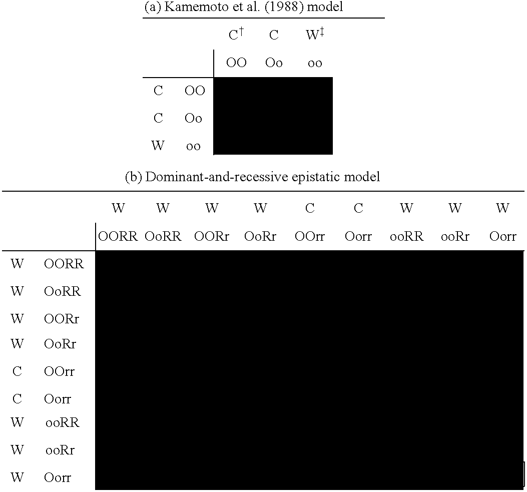

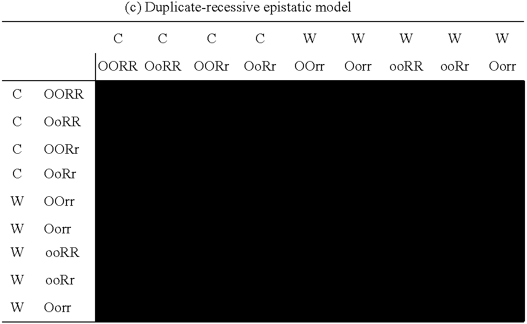

C^{†L , W}^{‡} indicate colored- and white-spathed, respectively.
The ratios given are colored:white.

TABLE 39
The expected ratios for colored:white-spathed colors in segregating
populations of white × white, white × color and color × color crosses in
anthurium, based on three genetic models^{a} along with the proportion of
crosses giving the specific ratios.

| | Genetic model | | | | | |
|---|---|---|---|---|---|---|
| | Kamemoto | | Dom. & recessive | | Dupl. recessive | |
| Spathe color | Expected | Proportion | Expected | Proportion | Expected | Proportion |
| Of crosses | ratio | crosses | ratio | crosses | ratio | Crosses |
| White × white | 0:1 | 1.00 | 0:1 | 0.76 | 0:1 | 0.68 |
| | | | 1:3 | 0.14 | 1:1 | 0.16 |
| | | | 3:13 | 0.02 | 1:0 | 0.08 |
| | | | 1:7 | 0.04 | 1:3 | 0.08 |
| | | | 1:1 | 0.04 | | |
| White × color | 1:0 | 0.50 | 0:1 | 0.43 | 1:1 | 0.40 |
| | 1:1 | 0.50 | 1:1 | 0.36 | 1:0 | 0.35 |
| | | | 3:5 | 0.07 | 3:1 | 0.10 |
| | | | 1:3 | 0.07 | 3:5 | 0.10 |
| | | | 1:0 | 0.07 | 1:3 | 0.05 |
| Color × color | 1:0 | 0.67 | 1:0 | 0.75 | 9:7 | 0.06 |
| | 3:1 | 0.33 | 3:1 | 0.25 | 3:1 | 0.38 |
| | | | | | 1:0 | 0.56 |

^{a}The ratios presented are plausible ratios based on the Kamemoto et al. (1988) model, dominant-and-recessive epistasis model or duplicate-recessive epistasis model.

TABLE 40
The most plausible observed ratios for color:white spathe colors
in segregating populations of white × white, white × color and
color × color crosses in anthurium, based on three genetic models^{a},
along with the proportion of crosses giving the specific ratios.

| | | |
|---|---|---|
| Spathe color | Observed | Prop.^{b} |

| of crosses | Ratio | No. | crosses |
|---|---|---|---|
| White × white | 1:1 | 2 | 0.33 |
| | 1:3 | 1/3 | 0.17/0.50 |
| | 1:7 | 1/2 | 0.17/0.33 |
| | 3:13 | 1 | 0.17 |
| | | 6 | |
| White × color | 1:0 | 23 | 0.68 |
| | 1:1 | 4 | 0.12 |
| | 3:1 | 5 | 0.15 |
| | 3:5 | 2 | 0.06 |
| | 1:3 | 0 | 0.00 |
| | 0:1 | 0 | 0.00 |
| | | 34 | |
| Color × color | 1:0 | 31 | 0.82 |
| | 3:1 | 7 | 0.18 |
| | 9:7 | 0 | 0.00 |
| | | 38 | |

^{a}The ratios presented are plausible ratios based on the Kamemoto et al. (1988) model, dominant-and-recessive epistasis model or duplicate-recessive epistasis model.
^{b}proportion.

TABLE 41
Progeny spathe color segregation ratios (color:white) for 6 white- × white-
spathed anthurium cultivar crosses.
White × White crosses

| | | | | | Fitted | | |
|---|---|---|---|---|---|---|---|
| Female | Male | Color | White | Total | Ratio | N² | Probability |
| Bianco | Pierrot | 19 | 24 | 43 | 1:1 | 0.372 | 0.50-0.70 |
| Pierrot | Priscilla | 15 | 34 | 49 | 1:3 | 0.551 | 0.30-0.50 |
| Local Mina White | Cuba | 10 | 67 | 77 | 1:7; 1:3 | 0.002; | 0.95-0.975; |
| | | | | | | 5.305 | 0.02-0.025 |
| Ibara | Cross 4044 | 4 | 30 | 34 | 1:7; 1:3 | 0.017; | 0.80-0.90; |
| | | | | | | 2.509 | 0.10-0.20 |
| Linda | Midori | 32 | 40 | 72 | 1:1 | 0.680 | 0.30-0.50 |
| Champagne | Pierrot | 16 | 75 | 91 | 3:13; | 0.018; | 0.80-0.90; |
| | | | | | 1:3 | 2.289 | 0.10-0.20 |

TABLE 42
Progeny spathe color segregation ratios (color:white) for 9 orange-,
coral- × white-spathed anthurium cultivar crosses.
Orange/Coral × White crosses

| | | | | | Fitted | | |
|---|---|---|---|---|---|---|---|
| Female | Male | Color | White | Total | Ratio | N² | Probability |
| Midori (w)^{a} | Venus (c)^{b} | 20 | 0 | 20 | 1:0 | 0.000 | 1.00 |
| Fantasia (w) | Venus (c) | 32 | 2 | 34 | 1:0 | | |
| Champagne (w) | Venus (c) | 51 | 0 | 51 | 1:0 | 0.000 | 1.00 |
| Pierrot (w) | Venus (c) | 52 | 0 | 52 | 1:0 | 0.000 | 1.00 |
| Local Orange (o) | Cuba (w) | 104 | 0 | 104 | 1:0 | 0.000 | 1.00 |
| Acropolis (w) | Hawaii (o)^{c} | 72 | 5 | 77 | 1:0 | | |
| Fantasia (w) | Hawaii (o) | 32 | 3 | 35 | 1:0 | | |
| Pierrot (w) | Local Orange (o) | 50 | 9 | 59 | 1:0 | | |
| Champagne (w) | Hawaii (o) | 31 | 3 | 34 | 1:0 | | |

^{a,b,c}white-, coral-, and orange-spathe cultivars, respectively.

TABLE 43
Progeny spathe color segregation ratios (color:white) for 12 pink- × white-
spathed anthurium cultivar crosses.
Pink × White Crosses

| | | | | | Fitted | | |
|---|---|---|---|---|---|---|---|
| Female | Male | Color | White | Total | Ratio | N² | Probability |
| Laguna (w)^{a} | Sonate (p)^{b} | 27 | 13 | 40 | 3:1 | 0.833 | 0.30-0.50 |
| Champagne (w) | Victoria (p) | 17 | 13 | 30 | 1:1 | 0.300 | 0.50-0.70 |
| Champagne (w) | Sonate (p) | 15 | 7 | 22 | 3:1 | 0.243 | 0.50-0.70 |
| LWP^{c} (p) | Pierrot (w) | 35 | 64 | 99 | 3:5 | 0.115 | 0.70-0.75 |
| Cuba (w) | Local Pink (p) | 16 | 28 | 44 | 3:5 | 0.000 | 1.00 |
| Laguna (w) | Local Pink (p) | 16 | 14 | 30 | 1:1 | 0.034 | 0.80-0.90 |
| Champagne (w) | Cross 3962 (p) | 46 | 4 | 50 | 1:0 | | |
| New Pink 2 (p) | Pierrot (w) | 22 | 1 | 23 | 1:0 | | |
| Cuba (w) | Pastora (p) | 35 | 3 | 38 | 1:0 | | |
| Champagne (w) | Lydia (p) | 16 | 5 | 21 | 3:1 | 0.016 | 0.80-0.90 |
| Rosa (p) | Acropolis (w) | 11 | 14 | 25 | 1:1 | 0.160 | 0.50-0.70 |
| Acropolis (w) | Victoria (p) | 40 | 38 | 78 | 1:1 | 0.012 | 0.90-0.95 |

^{a, b, c}white, pink and ‘Local Whitish Pink’, respectively.

TABLE 44
Progeny spathe color segregation ratios (color:white) for 13 pink- × white-
spathed anthurium cultivar crosses.
Red × White Crosses

| | | | | | Fitted | | |
|---|---|---|---|---|---|---|---|
| Female | Male | Color | White | Total | Ratio | N² | Probability |
| Midori (w)^{a} | Sweety (r)^{b} | 25 | 11 | 36 | 3:1 | 0.333 | 0.50-0.70 |
| Laguna (w) | LMR^{f} | 37 | 0 | 37 | 1:0 | 0.000 | 1.00 |
| Cuba (w) | Success (r) | 37 | 8 | 45 | 3:1 | 0.896 | 0.30-0.50 |
| LMR^{c} (r) | Cuba (w) | 45 | 1 | 46 | 1:0 | | |
| Mirjam (r) | Cuba (w) | 26 | 0 | 26 | 1:0 | 0.000 | 1.00 |
| Mirjam (r) | Ibara (w) | 33 | 0 | 33 | 1:0 | 0.000 | 1.00 |
| Gloria (r) | Pierrot (w) | 99 | 0 | 99 | 1:0 | 0.000 | 1.00 |
| Champagne (w) | Gloria (r) | 23 | 0 | 23 | 1:0 | 0.000 | 1.00 |
| Acropolis (w) | Gloria (r) | 39 | 1 | 40 | 1:0 | | |
| Mirjam (r) | Tequila (w) | 17 | 1 | 18 | 1:0 | | |
| Champagne (w) | Honduras (r) | 103 | 8 | 111 | 1:0 | | |
| Honduras (r) | Cuba (w) | 26 | 1 | 27 | 1:0 | | |
| Cross 4038 (r) | Cross 442 (w) | 28 | 0 | 28 | 1:0 | 0.000 | 1.00 |

^{a,b}white- and red-spathed, respectively.
^{c}= Local Mina Red.

TABLE 45
Progeny spathe color segregation ratios (color:white) for 8 red-, pink- ×
orange-, coral-spathed anthurium cultivar crosses.
Red/Pink × Orange/Coral crosses

| | | | | | Fitted | | |
|---|---|---|---|---|---|---|---|
| Female | Male | Color | White | Total | Ratio | N² | Probability |
| Local Orange (o)^{a} | Gloria (r)^{b} | 18 | 0 | 18 | 1:0 | 0.000 | 1.00 |
| Mirjam (r) | Hawaii (o) | 11 | 0 | 11 | 1:0 | 0.000 | 1.00 |
| Kalapana (r) | Hawaii (o) | 23 | 0 | 23 | 1:0 | 0.000 | 1.00 |
| Local Orange (o) | Honduras (r) | 95 | 0 | 95 | 1:0 | 0.000 | 1.00 |
| Sonate (p)^{c} | Terra (o) | 22 | 6 | 28 | 3:1 | 0.048 | 0.80-0.90 |
| Senator (c)^{d} | Victoria (p) | 22 | 7 | 29 | 3:1 | 0.012 | 0.90-0.95 |
| Senator (c) | New obake (p) | 26 | 8 | 34 | 3:1 | 0.000 | 1.00 |
| Cross 2794 (p) | Marissa (c) | 45 | 5 | 50 | 1:0 | | |

^{a,b,c,d}orange-, red-, pink-, and coral-spathed cultivars, respectively.

TABLE 46
Progeny spathe color segregation ratios (color:white) for 8 pink- × pink-
spathed anthurium cultivar crosses.
Pink × Pink Crosses

| Female | Male | Color | White | Total | Fitted Ratio | N² | Probability |
|---|---|---|---|---|---|---|---|
| Cross 4414 | Cross 3858 | 34 | 1 | 35 | 1:0 | | |
| Lydia | New Obake | 59 | 5 | 64 | 1:0 | | |
| Cross 2918 | Cross 800 | 31 | 0 | 31 | 1:0 | 0.000 | 1.00 |
| Spirit | Lydia | 19 | 2 | 21 | 1:0 | | |
| Cross 4062 | Local Mina Pink | 28 | 2 | 30 | 1:0 | | |
| Lydia | Cross 3962 | 84 | 1 | 85 | 1:0 | | |
| Lydia | Local Pink | 31 | 2 | 33 | 1:0 | | |
| Cross 2520 | Cross 3942 | 22 | 0 | 22 | 1:0 | 0.000 | 1.00 |

TABLE 47
Progeny spathe color segregation ratios (color:white) for 11 red- × pink-
spathed anthurium cultivar crosses.
Red × Pink Crosses

| Female | Male | Color | White | Total | Fitted Ratio | N² | Probability |
|---|---|---|---|---|---|---|---|
| Rosa (p)^{a} | Local Mina Red (r)^{b} | 25 | 0 | 25 | 1:0 | 0.000 | 1.00 |
| Gloria (r) | Local Mina Pink (p) | 39 | 0 | 39 | 1:0 | 0.000 | 1.00 |
| Cross 3023 (r) | Cross 2135 (p) | 75 | 0 | 75 | 1:0 | 0.000 | 1.00 |
| Alexis (r) | Cumbia (p) | 68 | 7 | 75 | 1:0 | | |
| Lydia (p) | Honduras (r) | 22 | 0 | 22 | 1:0 | 0.000 | 1.00 |
| Cross 868 (r) | Cross 3476 (p) | 40 | 11 | 51 | 3:1 | 0.164 | 0.50-0.70 |
| Alexis (r) | Sonate (p) | 21 | 7 | 28 | 3:1 | 0.000 | 1.00 |
| Cross 3071 (p) | Cross 3864 (r) | 25 | 5 | 30 | 3:1 | 0.711 | 0.30-0.50 |
| Alexis (r) | Local Pink (p) | 35 | 21 | 56 | 3:1 | 4.024 | 0.025-0.05 |
| Honduras (r) | Cross 3962 (p) | 27 | 0 | 27 | 1:0 | 0.000 | 1.00 |
| Cross 2679 (p) | Cross 4038 (r) | 19 | 2 | 21 | 1:0 | | |

^{a,b}pink- and red-spathe colors, respectively.

TABLE 48
Progeny spathe color segregation ratios (color:white) for 8 red- × red-
spathed anthurium cultivar crosses.
Red × Red Crosses

| | | | | | Fitted | | |
|---|---|---|---|---|---|---|---|
| Female | Male | Color | White | Total | Ratio | N² | Probability |
| Local Mina Red | Gloria | 24 | 0 | 24 | 1:0 | 0.000 | 1.00 |
| Cross 163 | Cross 793 | 25 | 0 | 25 | 1:0 | 0.000 | 1.00 |
| Kalapana | Local Mina Red | 30 | 0 | 30 | 1:0 | 0.000 | 1.00 |
| Mirjam | Kalapana | 45 | 0 | 45 | 1:0 | 0.000 | 1.00 |
| Mirjam | Gloria | 43 | 0 | 43 | 1:0 | 0.000 | 1.00 |
| Mirjam | Alexis | 34 | 0 | 34 | 1:0 | 0.000 | 1.00 |
| Gloria | Honduras | 40 | 3 | 43 | 1:0 | | |
| Honduras | Local Mina Red | 91 | 0 | 91 | 1:0 | 0.000 | 1.00 |

TABLE 49
Progeny spathe color segregation ratios (color:white) for 3 orange- × orange-
spathed anthurium cultivar crosses.
Orange, coral × Orange Crosses

| Female | Male | Color | White | Total | Fitted Ratio | N² | Probability |
|---|---|---|---|---|---|---|---|
| Local Orange (o) | Terra (o) | 21 | 0 | 21 | 1:0 | 0.000 | 1.00 |
| Senator (c) | Hawaii (o) | 79 | 6 | 85 | 1:0 | | |
| Local Orange (o) | Venus (c) | 44 | 0 | 44 | 1:0 | 0.000 | 1.00 |

The colored×white crosses gave the observed ratios 1:0, 1:1, 3:1 and 3:5 (Table 40). Although all these ratios are plausible under the ‘duplicate-recessive’ and ‘dominant-and-recessive’ epistasis models, only the ratios 1:1 and 1:0 are possible under Kamemoto's model (Table 39). These results further indicate that the Kamemoto model may not be able to explain the segregation of spathe colors in modern anthurium cultivars. Theoretical analysis (Table 38) shows that the 1:0, colored:white ratio, is the most frequent result to be expected among the colored×white crosses under the ‘duplicate-recessive’ model (Table 39), which was indeed the case in this study (Table 40). On the other hand, the 1:0, colored:white ratio, for colored×white crosses is theoretically expected to have a low frequency, 0.07 (Table 39), based on the ‘dominant-and-recessive’epistasis model, which was not observed in this study. Hence the results from the colored×white crosses favor the ‘duplicate-recessive’ epistasis model.

Of the 23 colored×white crosses that gave a 1:0 (colored:white) segregation ratio, only 10 gave a perfect fit (Table 42 to Table 44). In the other cases, one to a few whites were found against a large number of colored. These whites occurred at such low frequencies that they can be considered contaminants or mislabeled plants. Similar results were also obtained in the study by Kamemoto et al. (1988, supra).

The colored×colored crosses (Table 40) gave the ratios 1:0, 3:1 and 9:7, of which the 1:0 ratio is by far the most frequent (80%). This is the expectation with all three genetic models (1:0, colored:white ratio for colored×colored crosses). Among the three ratios observed (Table 45-Table 49), the former two are plausible with all the three genetic models, whereas the latter is possible only with the ‘duplicate-recessive’ epistasis model. This further provides support for the ‘duplicate-recessive’ epistasis model. Twenty of the 31 colored×colored crosses that produced a 1 color:0 white ratio gave a perfect fit, whereas the remainder had one or a few white contaminants (Table 45-Table 49).

In general, a ‘duplicate-recessive’ epistasis model can be used to explain the segregation (colored:white) patterns of almost all of the crosses investigated in this study. The only ratio that did not fit the model was the 1:7 (C:W) obtained with one of the white×white crosses (‘Local Mina White×Cuba’). This cross however, did not have very large progeny numbers.

Although, the 0:1 (color:white) ratio was the most likely one for the white×white crosses, none of the six crosses gave this ratio. Incidentally, this was the only ratio observed by Kamemoto et al. (1988, supra) for white×white crosses. This surprising result can be explained using the ‘duplicate-recessive’ epistasis model. It is noteworthy that the 0:1, colored:white ratio, is obtained only when either gene ‘O’ or ‘R’ are in the homozygous recessive state in both the parents based on the ‘duplicate-recessive’ model (Table 38). In modern anthurium cultivars, which are based on interspecific hybridization and are a result of frequent intercrossing between various colors, it is highly probable that the color loci are heterozygous at least in one of the parents, accounting for this unusual result.

It was also noteworthy that the 1:0 ratio (C:W) for the colored×white crosses was predominantly seen in the red×white crosses (Table 44), whereas all the ratios were equally frequent among the pink×white crosses (Table 43). One can observe, based on Table 38c, that the 1:0 ratios between colored and white crosses were predominantly obtained when the colored have a genotype of ‘OORR’. Prior work by Kamemoto et al. (1988, supra) suggested a dosage effect of genes and ‘O’ and ‘M’, which in their heterozygous forms give pinks and in their homozygous dominant states give reds. Hence, the heterozygous nature of ‘O’ may explain why 1:0 ratio was not common in pink×white crosses as with red×white ones.

Testing Kamemoto's Monogenic Model for the Segregation of Colors

Table 80 shows the expected ratios for the segregation of spathe colors in the red- and orange-groups as described by Kamemoto et al. (1988, supra). This follows a monogenic inheritance with complete dominance The gene is denoted as ‘M’, with genotypes ‘MM’ and ‘Mm’ resulting in the red-group, and genotype ‘mm’ resulting in the orange-group.

Table 81 to Table 89 show the observed and expected phenotypic ratios as well as chi-squared tests for the various crosses. Genotypes were assigned to each cultivar by trial and error based on the number of crosses each cultivar was involved in. As the colored-spathed phenotypes were further subdivided into reds and orange, the numbers were considerably smaller than for the colored vs. white analysis. However, the results showed a general agreement throughout with the Kamemoto model.

Of the 78 crosses investigated, 53 gave good fits to the proposed ratios and another 10 had just one spurious orange, which was most likely due to contamination or misclassification. Hence, in total approximately 81% of the crosses fitted the Kamemoto model very well. The poor fits with the remainder of the crosses are most likely due to misclassification as the lighter pinks and corals can sometimes be mistaken for each other.

Based on the genotype assignment to cultivars (Table 90), white cultivars could be ‘mm’, ‘Mm’, and ‘NM’, whereas all coral and orange cultivars were ‘mm’ Pink and red cultivars (Table 90) were assigned either ‘NM’ or ‘Mm’.

TABLE 80
Segregation patterns for expected progeny outcomes for red-
and orange-spathed anthurium crosses based on the
proposed dominance model.
Red × Red crosses

| Red | Red | Red | Orange |
|---|---|---|---|
| MM | MM | 1 | 0 |
| MM | Mm | 1 | 0 |
| Mm | Mm | 3 | 1 |

Red × Orange crosses

| Red | Orange | Red | Orange |
|---|---|---|---|
| MM | mm | 1 | 0 |
| Mm | mm | 1 | 1 |

Orange × Orange crosses

| Orange | Orange | Red | Orange |
|---|---|---|---|
| mm | mm | 0 | 1 |

TABLE 81
Progeny spathe color segregation ratios (red:orange) for 6 white- × white-
spathed anthurium cultivar crosses based on the M gene.
White × White crosses

| | | | | | | | Fitted | | |
|---|---|---|---|---|---|---|---|---|---|
| Female | Genotype | Male | Genotype | Red | Orange | Total | Ratio | N² | Probability |
| Bianco | MM | Pierrot | Mm | 19 | 0 | 19 | 1:0 | 0.000 | 1.00 |
| Pierrot | Mm | Priscilla | MM | 15 | 0 | 15 | 1:0 | 0.000 | 1.00 |
| Local Mina White | MM | Cuba | Mm | 10 | 0 | 10 | 1:0 | 0.000 | 1.00 |
| Ibara | Mm | Cross 4044 | MM | 4 | 0 | 4 | 1:0 | 0.000 | 1.00 |
| Linda | MM | Midori | mm | 29 | 3 | 32 | 1:0 | | |
| Champagne | Mm | Pierrot | Mm | 13 | 3 | 16 | 3:1 | 0.084 | 0.75-0.80 |

TABLE 82
Progeny spathe color segregation ratios (red:orange) for 9 orange-/coral ×
white-spathed anthurium cultivar crosses based on the M gene.

| | | | | | | | Fitted | | |
|---|---|---|---|---|---|---|---|---|---|
| Female | Genotype | Male | Genotype | Red | Orange | Total | Ratio | N² | Probability |
| Midori (w)^{a} | mm | Venus (c)^{b} | mm | 0 | 20 | 20 | 0:1 | 0.000 | 1.00 |
| Fantasia (w) | MM | Venus (c) | mm | 31 | 1 | 32 | 1:0 | | |
| Champagne (w) | Mm | Venus (c) | mm | 29 | 22 | 51 | 1:1 | 0.706 | 0.30-0.50 |
| Pierrot (w) | Mm | Venus (c) | mm | 34 | 18 | 52 | 1:1 | 4.326 | 0.025-0.05 |
| Local Orange (o) | mm | Cuba (w) | Mm | 55 | 49 | 104 | 1:1 | 0.240 | 0.50-0.70 |
| Acropolis (w) | mm | Hawaii (o)^{c} | mm | 6 | 66 | 72 | 0:1 | | |
| Fantasia (w) | MM | Hawaii (o) | mm | 31 | 1 | 32 | 1:0 | | |
| Pierrot (w) | Mm | Local Orange (o) | mm | 19 | 31 | 50 | 1:1 | 2.420 | 0.10-0.20 |
| Champagne (w) | Mm | Hawaii (o) | mm | 16 | 15 | 31 | 1:1 | 0.000 | 1.00 |

^{a,b,c}white-, coral-, and orange-spathe colors, respectively.

TABLE 83
Progeny spathe color segregation ratios (red:orange) for 12 pink- × white-
spathed anthurium cultivar crosses based on the M gene.
Pink × White Crosses

| | | | | | | | Fitted | | |
|---|---|---|---|---|---|---|---|---|---|
| Female | Genotype | Male | Genotype | Red | Orange | Total | Ratio | N² | Probability |
| Laguna (w)^{a} | Mm | Sonate (p)^{b} | MM | 26 | 1 | 27 | 1:0 | | |
| Champagne (w) | Mm | Victoria (p) | MM | 16 | 1 | 17 | 1:0 | | |
| Champagne (w) | Mm | Sonate (p) | MM | 14 | 1 | 15 | 1:0 | | |
| L W P^{c}(p) | MM | Pierrot (w) | Mm | 35 | 0 | 35 | 1:0 | 0.000 | 1.00 |
| Cuba (w) | Mm | Loc Pink^{d} (p) | Mm | 12 | 4 | 16 | 3:1 | 0.000 | 1.00 |
| Laguna (w) | Mm | Loc Pink (p) | Mm | 11 | 5 | 16 | 3:1 | 0.084 | 0.75-0.80 |
| Champagne (w) | Mm | Cr. 3962^{e} (p) | MM | 46 | 0 | 46 | 1:0 | 0.000 | 1.00 |
| New Pink 2 (p) | MM | Pierrot (w) | Mm | 21 | 1 | 22 | 1:0 | | |
| Cuba (w) | Mm | Pastora (p) | MM; Mm | 31 | 4 | 35 | 1:0; 3:1 | —; | —; 0.05-0.10 |
| | | | | | | | | 2.752 | |
| Champagne (w) | Mm | Lydia (p) | MM | 15 | 1 | 16 | 1:0 | | |
| Rosa (p) | MM | Acropolis (w) | mm | 10 | 1 | 11 | 1:0 | | |
| Acropolis (w) | mm | Victoria (p) | MM | 40 | 0 | 40 | 1:0 | 0.000 | 1.00 |

^{a,b}white- and pink-spathed, respectively.
^{c},^{d},^{e}= ‘Local Whitish Pink’, Local Pink and Cross 3962, respectively.

TABLE 84
Progeny spathe color segregation ratios (red:orange) for 13 red- × white-
spathed anthurium cultivar crosses based on the M gene.
Red × White Crosses

| | | | | | | | Fitted | | |
|---|---|---|---|---|---|---|---|---|---|
| Female | Genotype | Male | Genotype | Red | Orange | Total | Ratio | N² | Probability |
| Midori (w)^{a} | mm | Sweety (r)^{b} | Mm | 19 | 6 | 25 | 3:1 | 0.013 | 0.90-0.95 |
| Laguna (w) | Mm | LMR^{c} | Mm | 32 | 5 | 37 | 3:1 | 2.027 | 0.10-0.20 |
| Cuba (w) | Mm | Success (r) | Mm | 31 | 6 | 37 | 3:1 | 1.091 | 0.25-0.30 |
| LMR (r) | Mm | Cuba (w) | Mm | 40 | 5 | 45 | 3:1 | 3.919 | 0.025-0.05 |
| Mirjam (r) | Mm | Cuba (w) | Mm | 20 | 6 | 26 | 3:1 | 0.000 | 1.00 |
| Mirjam (r) | Mm | Ibara (w) | Mm | 27 | 6 | 33 | 3:1 | 0.495 | 0.30-0.50 |
| Gloria (r) | Mm | Pierrot (w) | Mm | 81 | 18 | 99 | 3:1 | 2.104 | 0.10-0.20 |
| Champ^{d} (w) | Mm | Gloria (r) | Mm | 17 | 6 | 23 | 3:1 | 0.015 | 0.90-0.95 |
| Acropolis (w) | mm | Gloria (r) | Mm | 27 | 12 | 39 | 1:1 | 5.026 | 0.02-0.025 |
| Mirjam (r) | Mm | Tequila (w) | Mm | 12 | 5 | 17 | 3:1 | 0.020 | 0.80-0.90 |
| Champ (w) | Mm | Honduras (r) | MM | 103 | 0 | 103 | 1:0 | 0.000 | 1.00 |
| Honduras (r) | MM | Cuba (w) | Mm | 26 | 0 | 26 | 1:0 | 0.000 | 1.00 |
| Cross 4038 (r) | MM, Mm | Cross 442 (w) | MM, Mm | 28 | 0 | 28 | 1:0 | 0.000 | 1.00 |

^{a,b}white- and red-spathed, respectively;
^{c}Local Mina Red;
^{d}Champagne.

TABLE 85
Progeny spathe color segregation ratios (red × orange) for 8 red-, pink- ×
orange-, coral-spathed anthurium cultivar crosses based on the M gene.
Red/Pink × Orange/Coral crosses

| | | | | | | | Fitted | | |
|---|---|---|---|---|---|---|---|---|---|
| Female | Genotype | Male | Genotype | Red | Orange | Total | Ratio | N² | Probability |
| Loc Orange (o)^{a} | mm | Gloria (r)^{b} | Mm | 8 | 10 | 18 | 1:1 | 0.056 | 0.80-0.90 |
| Mirjam (r) | Mm | Hawaii (o) | mm | 7 | 4 | 11 | 1:1 | 0.364 | 0.50-0.70 |
| Kalapana (r) | Mm | Hawaii (o) | mm | 15 | 8 | 23 | 1:1 | 1.566 | 0.20-0.25 |
| Loc Orange^{e} (o) | mm | Honduras (r) | MM | 91 | 4 | 95 | 1:0 | | |
| Sonate (p)^{c} | MM | Terra (o) | mm | 22 | 0 | 22 | 1:0 | 0.000 | 1.00 |
| Senator (c)^{d} | mm | Victoria (p) | MM | 20 | 2 | 22 | 1:0 | | |
| Senator (c) | mm | New obake (p) | MM | 24 | 2 | 26 | 1:0 | | |
| Cross 2794 (p) | MM | Marissa (c) | mm | 42 | 3 | 45 | 1:0 | | |

^{a,b,c,d}= orange, red, pink and coral spathe colors respectively.
^{e}Local Orange.

TABLE 86
Progeny spathe color segregation ratios (red:orange) for 8 pink- × pink-
spathed anthurium cultivar crosses based on the M gene.
Pink × Pink Crosses

| | | | | | | | Fitted | | |
|---|---|---|---|---|---|---|---|---|---|
| Female | Genotype | Male | Genotype | Red | Orange | Total | Ratio | N² | Prob.^{a} |
| Cross 4414 | MM, Mm | Cross 3858 | MM, Mm | 33 | 1 | 34 | 1:0 | | |
| Lydia | MM | New Obake | MM, Mm | 59 | 0 | 59 | 1:0 | 0.000 | 1.00 |
| Cross 2918 | MM, Mm | Cross 800 | MM, Mm | 31 | 0 | 31 | 1:0 | 0.000 | 1.00 |
| Spirit | MM, Mm | Lydia | MM | 19 | 0 | 19 | 1:0 | 0.000 | 1.00 |
| Cross 4062 | MM, Mm | Local Mina Pink | MM | 27 | 1 | 28 | 1:0 | | |
| Lydia | MM | Cross 3962 | MM, Mm | 84 | 0 | 84 | 1:0 | 0.000 | 1.00 |
| Lydia | MM | Local Pink | Mm | 28 | 3 | 31 | 1:0 | | |
| Cross 2520 | Mm | Cross 3942 | Mm | 17 | 5 | 22 | 3:1 | 0.000 | 1.00 |

^{a}Probability.

TABLE 87
Progeny spathe color segregation ratios (red:orange) for 3 orange- × orange-
spathed anthurium cultivar crosses based on the M gene.
Coral, Orange × Orange Crosses

| | | | | | | | Fitted | | |
|---|---|---|---|---|---|---|---|---|---|
| Female | Genotype | Male | Genotype | Red | Orange | Total | Ratio | N² | Probability |
| Local Orange (o) | mm | Terra (o) | mm | 0 | 21 | 21 | 0:1 | 0.000 | 1.00 |
| Senator (c) | mm | Hawaii (o) | mm | 10 | 69 | 79 | 0:1 | | |
| Local Orange (o) | mm | Venus (c) | mm | 2 | 42 | 44 | 0:1 | | |

TABLE 88
Progeny spathe color segregation ratios (red:orange) for 11 red- × pink-
spathed anthurium cultivar crosses based on the M gene.
Red × Pink Crosses

| | | | | | | | Fitted | | |
|---|---|---|---|---|---|---|---|---|---|
| Female | Genotype | Male | Genotype | Red | Orange | Total | Ratio | N² | Prob.^{c} |
| Rosa (p)^{a} | MM | LMR^{d} (r)b | Mm | 25 | 0 | 25 | 1:0 | 0.000 | 1.00 |
| Gloria (r) | Mm | LMP^{e}(p) | MM | 39 | 0 | 39 | 1:0 | 0.000 | 1.00 |
| Cross 3023 (r) | Mm | Cross 2135 (p) | Mm | 61 | 14 | 75 | 3:1 | 1.284 | 0.25-0.30 |
| Alexis (r) | MM | Cumbia (p) | MM, Mm | 65 | 3 | 68 | 1:0 | | |
| Lydia (p) | MM | Honduras (r) | MM | 22 | 0 | 22 | 1:0 | 0.000 | 1.00 |
| Cross 868 (r) | Mm | Cross 3476 (p) | Mm | 33 | 7 | 40 | 3:1 | 0.833 | 0.30-0.50 |
| Alexis (r) | MM | Sonate (p) | MM | 18 | 3 | 21 | 1:0 | | |
| Cross 3071 (p) | MM, Mm | Cross 3864 (r) | MM, Mm | 25 | 0 | 25 | 1:0 | 0.000 | 1.00 |
| Alexis (r) | MM | Local Pink (p) | Mm | 30 | 5 | 35 | 1:0 | | |
| Honduras (r) | MM | Cross 3962 (p) | MM, Mm | 27 | 0 | 27 | 1:0 | 0.000 | 1.00 |
| Cross 2679 (p) | MM, Mm | Cross 4038 (r) | MM, Mm | 19 | 0 | 19 | 1:0 | 0.000 | 1.00 |

^{a,b}= pink- and red-spathed cultivars, respectively.
^{c}Probability.
^{d,e}= ‘Local Mina Red’ and ‘Local Mina Pink’, respectively.

TABLE 89
Progeny spathe color segregation ratios (red:orange) for 8 red- × red-
spathed anthurium cultivar crosses based on the M gene.
Red × Red Crosses

| | | | | | | | Fitted | | |
|---|---|---|---|---|---|---|---|---|---|
| Female | Genotype | Male | Genotype | Red | Orange | Total | Ratio | N² | Prob.^{a} |
| LMR^{b} | Mm | Gloria | Mm | 16 | 8 | 24 | 3:1 | 0.500 | 0.30-0.50 |
| Cross 163 | Mm | Cross 793 | Mm | 19 | 6 | 25 | 3:1 | 0.013 | 0.90-0.95 |
| Kalapana | Mm | LMR | Mm | 26 | 4 | 30 | 3:1 | 1.600 | 0.20-0.25 |
| Mirjam | Mm | Kalapana | Mm | 36 | 9 | 45 | 3:1 | 0.363 | 0.50-0.70 |
| Mirjam | Mm | Gloria | Mm | 36 | 7 | 43 | 3:1 | 1.311 | 0.25-0.30 |
| Mirjam | Mm | Alexis | MM | 31 | 3 | 34 | 1:0 | | |
| Gloria | Mm | Honduras | MM | 40 | 0 | 40 | 1:0 | 0.000 | 1.00 |
| Honduras | MM | LMR | Mm | 91 | 0 | 91 | 1:0 | 0.000 | 1.00 |

^{a}Probability.
^{b}‘Local Mina Red’.

TABLE 90
Plausible genotype/s based on the m gene for anthurium cultivars.

| Spathe color | Cultivar | Genotype |
|---|---|---|
| White | Acropolis | mm |
| | Bianco | MM |
| | Champagne | Mm |
| | Cross 442 | MM, Mm |
| | Cuba | Mm |
| | Fantasia | MM |
| | Ibara | Mm |
| | Laguna | Mm |
| | Linda | MM |
| | Local Mina White | MM |
| | Midori | mm |
| | Pierrot | Mm |
| | Priscilla | MM |
| | Tequila | Mm |
| | Cross 4044 | MM |
| Coral | Marissa | mm |
| | Senator | mm |
| | Venus | mm |
| Orange | Hawaii | mm |
| | Local Orange | mm |
| | Terra | mm |
| Pink | Cross 2135 | Mm |
| | Cross 2520 | Mm |
| | Cross 2794 | MM |
| | Cross 2918 | MM, Mm |
| | Cross 3071 | MM, Mm |
| | Cross 3476 | Mm |
| | Cross 3858 | MM, Mm |
| | Cross 3942 | Mm |
| | Cross 3962 | MM, Mm |
| | Cross 4062 | MM, Mm |
| | Cross 4414 | MM, Mm |
| | Cross 800 | MM, Mm |
| | Cross 93 | MM, Mm |
| | Cumbia | MM, Mm |
| Pink | Local Mina Pink | MM |
| | Local Pink | Mm |
| | Local Whitish Pink | MM |
| | Lydia | MM |
| | New Obake | MM, Mm |
| | New Pink 2 | MM |
| | Pastora | MM; Mm |
| | Rosa | MM |
| | Sonate | MM |
| | Spirit | MM, Mm |
| | Victoria | MM |
| Red | Alexis | MM |
| | Cross 163 | Mm |
| | Cross 3023 | MM, Mm |
| | Cross 3864 | MM, Mm |
| | Cross 4038 | MM, Mm |
| | Cross 793 | Mm |
| | Cross 868 | Mm |
| | Gloria | Mm |
| | Honduras | MM |
| | Kalapana | Mm |
| | Local Mina Red | Mm |
| | Mirjam | Mm |
| | Success | Mm |
| | Sweety | Mm |

Validity of the Kamemoto Model for Spathe Color

The data provided herein are compelling evidence that the two-gene model of Kamemoto et al. (1988, supra) does not explaining the segregation of colors and whites in progeny involving modern anthurium cultivars. Firstly, the white×white crosses gave ratios other than the expected 0:1 (C:W) ratio based on the Kamemoto model. Secondly, color×white crosses gave a wide range of ratios including 3:1, 3:5 and 1:3, and the color×color crosses gave among others a 9:7 ratio. These particular ratios are not expected based on the Kamemoto model. Thirdly, it was evident from the data that the dosage effect proposed for the ‘M’ gene to explain the difference between reds and pinks does not hold.

### An Alternative Genetic Model for the Inheritance of Spathe Color

In view of the inadequacies of the Kamemoto et al. model for inheritance of spathe color, a duplicate recessive model had been developed, which involves two genes designated ‘O’ and ‘R.’ This model explains the segregation of colors and white in progeny of most white×white crosses and all white×color and color×color crosses. Additional evidence for this model is derived from the ability to explain the uniform 1:0 ratio obtained for red×red and orange×orange crosses but various ratios for pink×pink crosses. The model can also explain the emergence of pinks and corals (described below).

The data also produced some unusual results. A few spurious whites emerged in some color×color crosses where, based on the duplicate recessive model involving ‘O’ and ‘R’ genes, only colored progeny were expected. These outliers may be contaminants, errors in labeling, or may indicate the involvement of other minor modifier genes. However, these minor deviations do not detract from the general agreement of the model with the data.

In the duplicate recessive model, either ‘R’ or ‘O’, or both ‘R’ and ‘O’ in the homozygous recessive forms will result in whites. In other words, the presence of genes ‘O’ and ‘R’ in the dominant forms (O-; R-) are required for colored genotypes to be obtained. The nature of the color, whether it belongs to the red group or the orange group, is determined by the status of the ‘M’ gene. For example, this gene in the dominant form (M-) results in the red group of colors whereas in its recessive form (‘mm’) gives the orange group of colors. The fact that the pinks and reds, belonging to the red group, could be either ‘Mm’ or ‘MM’ suggests that the ‘O’ and ‘R’ genes are somehow involved in their determination. To further understand this one needs to relate these genes to genes in the phenylpropanoid biosynthetic pathway.

### Molecular Basis for Control of Spathe Color

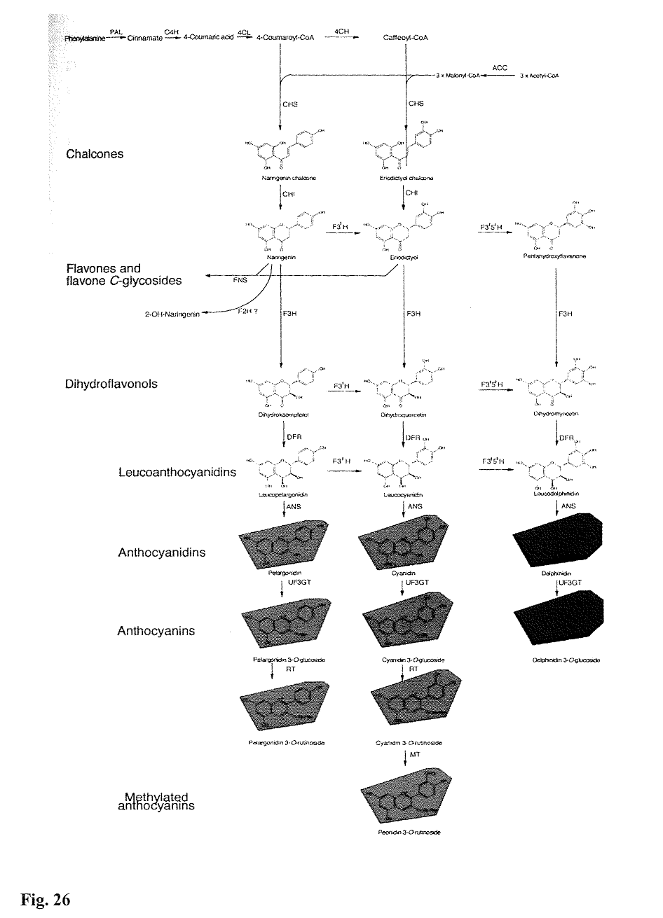

The genetic data from this study can be used to refine existing models for the molecular basis of spathe color in anthurium. FIG. 26 illustrates the phenylpropanoid pathway leading the various colored anthocyanin compounds. Two classes of genes are believed required for anthocyanin biosynthesis, the structural genes encoding the enzymes that directly participate in the formation of anthocyanins and other flavonoids, and the regulatory genes that control the transcription of structural genes.

Collette (2002) showed that the flavonoid biosynthetic genes in anthurium are regulated by two separate regulatory apparatuses, one simultaneously regulating a number of genes including CHS, F3H and ANS, and another specifically targeting the DFR gene. Anthocyanin production was found to be concomitant with DFR expression, indicating that DFR expression may serve as a point of regulation for anthocyanin biosynthesis. Collette (2002, supra) identified a candidate ‘Myb’ gene in anthurium as a possible transcriptional activator of the DFR gene. An unknown regulatory gene simultaneously suppressed the transcript levels of the structural genes CHS, F3H and ANS in the white anthurium cultivar, ‘Acropolis’ (Collette, 2002, supra).

The three genetic factors controlling spathe color are ‘M’, determining whether pelargonidin 3-rutinoside (orange) or cyanidin 3-rutinoside (red) are produced, and ‘O’ and ‘R’, controlling whether anthocyanins are produced. The most likely candidate for ‘M’ is F3′H, which converts dihydrokaempferol and leucopelargonidin to dihydroquercetin and leucocyanidin, respectively. The dominant ‘R’ gene could operate as a transcriptional activator of the regulatory cascade involving genes CHS, F3H and ANS, while the dominant ‘O’ gene may function as the transcriptional activator of DFR. Hence, either of the ‘R’ or ‘O’ genes in the homozygous recessive state may produce an inactive transcriptional activator leading to anthurium genotypes with white spathes. Therefore, when both ‘R’ and ‘O’ genes are in the recessive form the genes CHS, F3H, DFR and ANS will be suppressed, as was observed in ‘Acropolis’ (Collette, 2002, supra). Similarly, two petunia regulators ‘an1’ and/or ‘an11’ lead to a complete loss of expression of their target anthocyanin genes in all pigmented tissues and, consequently, to completely white petunia flowers. Regulatory genes in the anthocyanin biosynthetic pathway have also been reported in Antirrhinum and maize. Alternatively, the ‘O’ gene could be a structural gene (one of CHS, F3H, DFR and ANS) as suggested by Collette (2002, supra).

Anthurium flowers have an extended flower development period of 8-9 weeks (Collette, 2002, supra). The genes CHS, F3H and ANS are expressed constitutively, whereas DFR shows a distinct gene expression pattern, with a marked increase in transcript level at stage 3 followed by a significant decline between stages 4 and 6. The expression of CHS, F3H and ANS were quantitatively lower in the pinks compared to the reds and oranges at all stages of development. The DFR accumulation was also delayed to stage 6 in the pinks compared to stage 3 in the reds and oranges (Collette, 2002).

Genotyping the Parents for O and R

The plausible genotypes for the parental anthurium cultivars can be determined for each cross and further narrowed down by evaluating all the crosses of a particular cultivar with other cultivars. Table 91 summarizes the plausible genotypes determined for each cultivar. When the genotype at the ‘O’ and ‘R’ loci were ‘OORR’, then dark-red spathes were obtained when the ‘M’ locus was either ‘MM’ or ‘Mm’ and an orange spathe when the ‘M’ locus was homozygous recessive (‘mm’) However, when the ‘M’ locus was heterozygous (Mm) sometimes a deep-pink spathe was obtained. Lighter shades of red, such as with ‘Sweety’ and ‘Success’, were obtained when the genotype for the ‘O’ and ‘R’ loci were ‘OoRR’. Therefore, with the exception of the cultivar ‘Alexis’, it appears that reds are generally O_RR. In contrast, 8 of the 12 pink cultivars were ‘O-Rr²’. Where pinks were OoRR, such as ‘Local Pink,’ a deeper pink was obtained. This suggests that the ‘R’ locus appears to exert a strong dosage effect, depending on the number of dominant alleles it possesses. Similarly, the darker orange-spathed cultivars were ‘mmOORR’ and the corals were generally ‘mmOORr’, with the exception of cultivar, ‘Terra.’ More controlled crosses need to be carried out to verify these results. These analyses show that the Kamemoto model correctly predicted the ‘MM’ as belonging to the red group and ‘mm’ as belonging to the orange group. Whether the ‘Mm’ was red and orange or pink and coral depended on the status of the ‘O’ and ‘R’ loci.

The dosage effect of the ‘R’ gene can be explained by the proposed molecular model and supported by the study of Collette (2002). If the ‘R’ gene is as proposed, a transcriptional activator of the CHS, ANS, and F3H genes, and is expressed constitutively (Collette, 2002), then it will reduce the amount of substrate available for the ‘M’ gene (possible F3′H) and the ‘O’ gene (possibly the regulator of DFR gene) resulting in lower levels of anthocyanins. On the other hand, the ‘Myb’ regulator of the DFR gene (possibly ‘O’) is temporally controlled and is expressed specifically at stage 3 but decreases in intensity thereafter (Collette, 2002). Under substrate-limited conditions (Re), the differences between ‘MM’ and ‘Mm’ as well as ‘OO’ and

TABLE 91
The plausible genotypes, with respect to the hypothetical,
O, R and M loci, for anthurium cultivars belonging to
various flower color groups.

| Color | Cultivar | Genotype for O and R | Genotype for M |
|---|---|---|---|
| Coral | Senator | OORr, OoRr | mm |
| | Venus | OORR | mm |
| Orange | Hawaii | OORR | mm |
| | Local Orange | OORR | mm |
| | Terra | OoRr, OORr | mm |
| Pink | Cross 3962 | OORR | Mm |
| | Cumbia | OORR, OORr | Mm |
| | Local Whitish Pink | OoRr | MM |
| | Local Mina Pink | OoRr, OORr | MM |
| | Local Pink | OoRR | Mm |
| | Lydia | OORr | MM |
| | New obake | OoRR | Mm |
| | New Pink 2 | OORR | MM |
| | Pastora | OORR | Mm |
| | Rosa | OORr | MM |
| | Sonate | OORr | MM |
| | Spirit | OoRR | Mm |
| | Victoria | OORr | MM |
| Red | Alexis | OoRr, | MM |
| | Cross 163 | OoRR | Mm |
| | Cross 793 | OORr | Mm |
| | Gloria | OORR | Mm |
| | Honduras | OORR | MM |
| | Kalapana | OORR, OoRR | Mm |
| | Local Mina Red | OORR | Mm |
| | Mirjam | OORR | Mm |
| | Success | OoRR | Mm |
| | Sweety | OoRR | Mm |
| White | Acropolis | Oorr | mm |
| | Bianco | Oorr | MM |
| | Champagne | Oorr | Mm |
| | Cuba | ooRr | Mm |
| | Laguna | ooRr | Mm |
| | Linda | ooRr | MM |
| | Local Mina White | Oorr | MM |
| | Midori | Oorr | mm |
| | Pierrot | ooRr | Mm |
| | Priscilla | Oorr | MM |

‘Oo’ will not be as evident as when the ‘R’ gene is ‘RR’ (enzyme limited).

In contrast, genotypes ‘OOrr’, ‘Oorr’, ‘ooRr’ and ‘oorr’ possessed white spathes regardless of the composition of the ‘M’ gene, confirming the duplicate recessive epistasis model proposed. The acyanic mutant ‘Acropolis’ was the only genotype ascertained to be a triple recessive genotype, ‘mmoorr’, in all the crosses. This is in agreement with the findings of Collette (2002), who reported that the transcript levels of CHS, F3H, DFR and ANS genes were all suppressed in the cultivar, ‘Acropolis’. This suggested that both ‘O’ (responsible for suppression of DFR expression) and the ‘R’ (responsible for suppression of CHS, F3H, DFR and ANS expression) genes were in the recessive forms.

Generally, the whites belonging to the ‘O_rr’ group were consistently bright clean white in appearance. On the other hand, cultivars belonging to the ‘ooR-’ category varied from pure white as in the cultivar, ‘Pierrot’, to cream or white with areas of slight pink depending on the season as was observed with ‘Cuba’. If the ‘O’ gene is a regulator of the DFR as proposed, the apparent instability of whites can be explained by the molecular control mechanisms of DFR expression.

Collette (2002, supra) showed a diurnal fluctuation in DFR transcript levels, suggesting that DFR expression is linked to other processes within the plant apart from anthocyanin production. As reported in grapes (Gollop et al., 2002), it appears that more than one mechanism is involved in the control of anthurium DFR transcript levels. The first is an anthocyanin related mechanism, mediated through ‘Myb’ interaction. The second mechanism is a light mediated, signal transduction pathway that is reflected in the diurnal rhythm of DFR gene expression. The third possible mechanism is a calcium signal pathway. Hence, at any one time, the DFR expression being observed could be due to factors apart from activation by anthocyanin related ‘Mybs’. The multitude of regulatory cascades controlling the level of DFR transcripts may explain the influence of season and cultural practices on the degree of spathe whiteness in ‘ooR-’ genotypes.

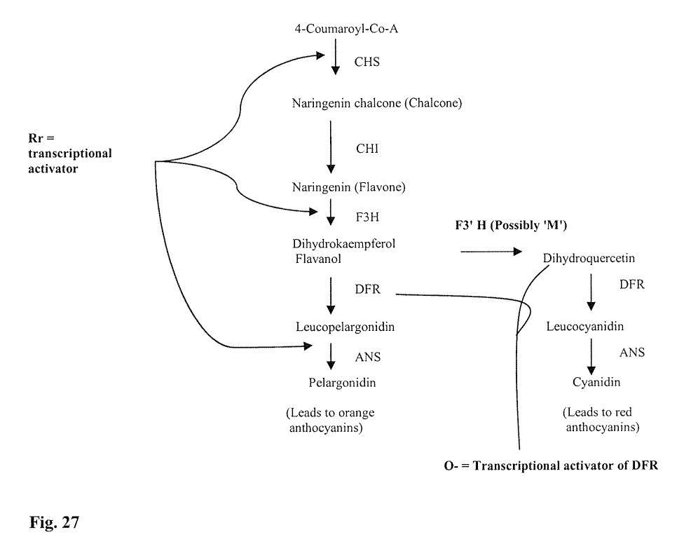

In conclusion, the proposed genetic model can largely explain the segregation ratios obtained in the 78 crosses and was able to reconcile molecular biological observations obtained on the expression of anthurium flavonoid genes by Collette (2002, supra), with field observations and genetic predictions. FIG. 27 summarizes the genetic/molecular model proposed. Further molecular biological characterization of genotypically characterized anthurium cultivars is needed to verify the proposed model.

The understanding of the molecular and genetic control model for anthurium flower color and the genotyping of parents will allow greater control in manipulating colors in breeding programs.

A three-gene model comprising the ‘M’, ‘O’ and ‘R’ genes was proposed to explain spathe color in anthurium. ‘M’ determines whether pelargonidin 3-rutinoside (orange) or cyanidin 3-rutinoside (red) is produced, and ‘O’ and ‘R’, control anthocyanin production. A duplicate-recessive model involving the ‘O’ and ‘R’ was able to explain the segregation of colors and white in progenies of most white×white crosses and all white×color and color×color crosses. In the duplicate-recessive model, either ‘R’ or ‘O’, or both ‘R’ and ‘O’ in the homozygous recessive forms will result in white-spathed anthuriums.

Productivity in anthurium is of polygenic inheritance. The narrow sense heritability for productivity is low suggesting that environment plays an important role in the determination of anthurium productivity.

### ADDITIONAL ASPECTS OF THE INVENTIONS

A number of promising genotypes were identified from the studies described herein. Productivity varied from 4.5-12 cut-flowers per year, with ‘Kalapana’, ‘Honduras’ and ‘Cuba’ being among the most productive. Although approximately 25% of the cultivars had high levels of resistance to X. campestris pv. dieffenbachiae at the foliar and systemic levels, only two cultivars (‘Acropolis’ and ‘Bianco’) combined resistance at both levels. Furthermore, the segregation analysis showed that most of the cultivars used in hybridization were heterozygous, and were therefore segregating for resistance to BB at the systemic level. Vase-life varied from 14 to 48 days and was largely determined by stomatal number and spathe color. Greens followed by whites had longer vase-lives than reds, pinks, orange and coral cultivars. However, within each color group, vase-life was determined by stomata number. The study identified eight cultivars (‘Champagne’, ‘Cuba’, ‘Fla Range’, ‘Honduras’, ‘Midori’, ‘Pierrot’, ‘Tequila’ and ‘Terra’) with vase-life well over three weeks.

The methods described herein enable strategic choice of parents so that the various characters (e.g., resistance to bacterial blight and vase-life) can be combined through recombination breeding. Furthermore, the genotyping of many parents for resistance and spathe color, described herein, enables selection of parents such that complementation of genes that control those characteristics can be achieved. Combining ability analysis also provides further information on selecting parents for hybridization. Character correlation showed little or no correlation between the horticulturally important characteristics investigated. These findings enable simultaneous improvement of these characteristics without selection of one negatively affecting another. The strong correlation between spathe length and width permits breeding to improve spathe size without changes to the shape.

### Screening Methods

Rapid and effective screening methods are described that are useful for determining foliar and systemic resistance to bacterial blight disease as well as vase-life. The optimized screening method for systemic resistance involves, for example, the injection of 100 μl of 10⁹ CFU/ml of X. campestris pv. dieffenbachiae cells into the cut-petiole of the second youngest leaf; and the optimized screening method for foliar resistance involved the vacuum-infiltration of 5.6 cm² leaf-discs with 10⁸ CFU/ml of X. campestris pv. dieffenbachiae cells. A useful measure to assess systemic resistance to bacterial blight disease was weeks to plant death, whereas that for foliar resistance was the time (days) taken for the lesion to cover the leaf disc. The optimized methods were shown to be repeatable, correlated to field resistance and environmentally stable. Further, they were not sensitive to plant age. These finding represent the first time that rapid screening methods have been developed and systematically tested for assessing their ability to determine levels of systemic and foliar resistance to bacterial blight disease in segregating anthurium populations.

The newly developed assessment techniques (at foliar and systemic levels) will for the first time allow the incorporation of resistance at both levels into the elite anthurium cultivars. The lack of anthurium cultivars that combine high levels of systemic and foliar resistance to bacterial blight disease suggests that the past breeding programs have not been successful in combining them.

Similarly, a rapid method for predicting vase-life in anthurium has been developed based on color and the simple assessment of stomatal number on the abaxial surface. Vase-life is an important quality attribute as the ornamental markets prefer cultivars with long vase-lives, over three weeks. Since the Caribbean islands export anthurium to the United States, Canada and Europe, growing cultivars in the Caribbean or other countries that are distant from their terminal markets with long vase-lives will ensure the retention of cut-flower freshness during transportation. The new method described herein enables breeders to incorporate longer vase-life into their breeding objectives.

### Mechanisms of Resistance to BB and Variation in Vase-Life

The mechanism of resistance to foliar infection was shown to be related to the ability of the pathogen to multiply in host tissue, whereas that for systemic resistance seems to be a function of the ability of the pathogen to invade the vascular system and transported systemically to the crown.

The physiological basis for variation in vase-life was investigated using a large number of anthurium cultivars. The study confirmed previous studies that the inability of certain cultivars to maintain water relations was the principal cause of loss of vase-life in anthurium cut-flowers. Time to incipient symptoms of spadix necrosis was found to be the best measure of vase-life. Investigation of a number of morpho-physiological characteristics showed that only abaxial stomata and hydathode number correlated to vase-life, although spathe thickness was negatively correlated to stomata number, and hence may have a role. In addition, the surprising finding was made that spathe color has an important role in the determination of vase-life.

### Genetic Basis for Resistance to Bacterial Blight, Color, and Productivity

The genetic basis of resistance to bacterial blight disease at both foliar and systemic levels was investigated. In addition, the genetic model for spathe color developed by Kamemoto et al. (1988, supra) was modified to explain segregation within crosses involving A. andraeanum (Hort.).

Two major genes, interacting in a duplicate recessive epistatic manner, were responsible for systemic resistance to bacterial blight disease. If the two genes were to be designated ‘A’ and ‘T’, then both genes in the recessive form or one in the recessive and the other in the heterozygous form would result in a resistant phenotype. This model was able to explain the segregation of susceptibility (plant death <10 weeks) and resistance (plant death is delayed longer than 10 weeks or does not occur), in crosses. However, there was a normal distribution for levels of resistance within the susceptible category, which ranged from 6-12 weeks. And similarly, there was continuous variation for levels of resistance from 12 weeks to no death. These observations suggest dosage effect of the major genes or the presence of other minor resistance genes that may be contributing to moderate levels of resistance/tolerance. Quantitative genetic analysis also indicated high broad sense heritability (96%) and a moderate (42.5%) narrow sense heritability for systemic resistance to bacterial blight disease indicating an equally important role for additive and non-additive genetic effects in the inheritance of the character.

The inheritance of foliar resistance to bacterial blight disease, in contrast, was polygenically inherited with predominantly additive genetic effects. The narrow sense heritability estimate was high (92%), which is similar to what has been observed with foliar resistance in various host-pathogen systems.

The Kamemoto et al. model for spathe color was not able to explain the segregation of most of the white×white and white×colored crosses. In contrast, a new model involving two dominant genes (‘O’ and ‘R’) interacting in a duplicate recessive manner can explain the ratio of colored:white spathes in wide range of crosses, and therefore can be used in breeding programs for propagating and developing plants having selected color. The segregation of colored spathes into the red group:orange group however, followed the Kamemoto model, and was influenced by an ‘M’ gene. The newly proposed trigenic model involving ‘M’, ‘O’ and ‘R’ genes was not only able to explain the segregation patterns observed in the studies described herein, but also the biochemical (Iwata et al., 1979, 1985) and molecular data (Collette, 2002) obtained from other studies. The model also accommodates an embodiment in which one of two regulatory genes, one (the ‘O’ gene) specifically controlling the expression of DFR gene and the other (the ‘R’ gene) transcriptionally activating a cascade of flavonoid pathway genes (CHS, F3H, ANS, and possibly others), in the recessive form, are responsible for white spathe colors. When both the genes are in the dominant form (O_R_), red and orange spathes are produced depending on whether the M gene (possibly F3′H) is dominant (M_) or recessive (mm), respectively. Dosage effect of the ‘O’ and possibly the ‘R’ gene may result in pinks and corals, depending on the status of the ‘M’ gene. Based on this genetic model, most of the parental cultivars have been given genotypes with respect to the O, R and M genes.

Productivity on the other hand had a moderate narrow sense heritability (37%), which indicates that it can be manipulated with moderate ease in breeding.

### Breeding Strategies

The development of elite cultivars with genetic resistance to bacterial blight disease has been an integral part of anthurium breeding programs in tropical countries such as U.S.A. (Hawaii) and the Caribbean. This is an important breeding objective because there are no effective pesticides or biological control measures capable of managing the disease or eliminating the inoculum, which persists in the form of symptomless infections.

The inability to fix complete resistance to bacterial blight disease into elite genotypes in the past may have been due to: (a) poor understanding of the genetic basis of resistance, and (b) lack of appropriate screening methods that can reliably detect genotypes with more resistant alleles. Cultivar, ‘Sweety’, which has both genes for systemic resistance in the homozygous state, is an ideal source of resistance to bacterial blight disease.

Understanding of the role of modifying factors such as dominance, epistasis and other minor genes on the inheritance of systemic resistance to bacterial blight disease as described herein permits the introduction of special strategies to accelerate the fixation of resistance in breeding populations. The screening method developed in this study (petiole inoculation) can identify plants with three to four resistant alleles, since they survive for over 20 weeks following inoculation. The systemically infected plants that may show latent symptoms can be easily detected by examination of portions of the petiole or leaf lamina for green fluorescence under a fluorescence microscope. Employing a scheme of ‘recurrent hybridization and selection’, for several breeding cycles, will help to eliminate the susceptible alleles from the population and therefore fix resistance using parental lines with several resistant alleles. Alternatively, progeny tests on surviving progeny plants (with ‘Rabb’ and ‘aaBb’ genotypes) can be done to eliminate genotypes harboring susceptible alleles or line breeding can be done. Yet another approach is to develop molecular markers for each of the two resistance genes, so that they can be selected for by their linkage to molecular markers. Whichever method is employed, it is generally important that the population created has a wide genetic base so that further improvement of other characteristics by cyclic selection is possible.

Similarly, high levels of foliar resistance to bacterial blight disease found in some elite anthurium cultivars, and the abundant transgressive segregation seen within progeny, indicates considerable promise in breeding for foliar resistance using the disc-inoculation method. The high narrow sense heritability for foliar resistance (92%) indicates that foliar resistance to bacterial blight disease can be constructed by recurrent mass selection methods, relatively easily.

Simultaneous selection for numerous characteristics can considerably reduce selection intensities for productivity and other horticultural characteristics. Therefore, the methods described herein can be used to undertake a population enhancement program for resistance (foliar and systemic) to bacterial blight disease, since this disease is a global problem. In some cases, the resistance-enhanced population is divided into various gene pools for the various colors. Such bacterial blight disease resistance-enhanced gene pools for particular spathe colors can then be subjected to population improvement for productivity by recurrent selection. For example, the red gene pool would have a fixed genotype, ‘MMOORR’.

Micropropagation methods for anthuriums have been well established through meristem culture or direct/indirect organogenesis. In some breeding programs, such methods are used to pursue clonal breeding. Clonal breeding enables the full exploitation of within progeny genetic variance for traits such as resistance to disease (e.g., bacterial blight disease), spathe color, and productivity. Furthermore, micropropagation permits rapid multiplication of the selections for productivity testing in plots, prior to initiating the next cycle of selection. This can therefore improve the breeding efficiency by reducing the length of the breeding cycle.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying resistance or tolerance of a plant to Xanthomonas infection, the method comprising
a. providing a target plant;
b. infecting the target plant with a fluorescent Xanthomonas expressing a heterologous fluorescent protein; and
c. detecting the level of fluorescent protein in the target plant, wherein a level of fluorescent protein in the target plant that is less than the level in a susceptible target plant indicates that the target plant is resistant to or tolerant of infection by the Xanthomonas.

2. The method of claim 1, wherein the target plant is an Anthurium.

3. The method of claim 1 or claim 2, wherein the Xanthomonas is Xanthomonas campestris pv. dieffenbachiae.

4. The method of claim 1, wherein the fluorescent protein is Green Fluorescent Protein.

5. The method of claim 1, wherein the Xanthomonas comprises p519 ngfp.

6. The method of claim 10, further comprising detecting foliar resistance.

7. The method of claim 6, wherein the foliar infection is determined using a leaf-disc vacuum-infiltration method.

8. The method of claim 1, wherein systemic infection is detected.

9. A method of identifying resistance or tolerance of a plant to Xanthomonas infection, the method comprising
a. providing a leaf sample from a target plant;
b. infecting the leaf sample with a Xanthomonas;
c. detecting Xanthomonas infection in the infected leaf sample; and
d. comparing the rate of infection to a reference to determine foliar resistance of the target plant.

10. A recombinant X. campestris comprising a fluorescent protein.

11. The recombinant X. campestris of claim 10, wherein the fluorescent protein is Green Fluorescent Protein.

12. The recombinant X. campestris of claim 10, wherein the X. campestris comprises p519 ngfp.

13. A method of predicting vase-life performance of an anthurium, the method comprising
a. providing a sample from an anthurium;
b. determining abaxial stomata number and spathe color in the anthurium sample; and
c. comparing abaxial stomata number and spathe color to a control or reference set, thereby predicting vase-life performance of the anthurium.

14. A method of predicting vase-life performance of an anthurium, the method comprising
a. providing an anthurium;
b. determining the average time for spadix necrosis of the anthurium; and
c. comparing the average time for spadix necrosis of the anthurium to a reference, thereby predicting the vase-life performance of the anthurium.

15. A plant breeding program comprising at least one of
a. assaying cultivars for resistance to Xanthomonas infection using the method of claim 1 and selecting resistant cultivars as a parental cultivar in the breeding program;
b. assaying cultivars for vase-life using the method of claim 13 or claim 14 and selecting a cultivar having a vase-life of at least three weeks as a parental cultivar in the breeding program; or
c. performing a. and b.
